(19) United States
(12) Patent Application Publication
(10) Pub. No.: US 2024/0315965 A1
(43) Pub. Date: Sep. 26, 2024

(54) COMPOSITIONS AND METHODS RELATED TO PHARMACEUTICAL EXCIPIENTS

(71) Applicant: NATURAL EXTRACTION SYSTEMS, LLC, Boulder, CO (US)

(72) Inventor: Douglas G. METCALF, Boulder, CO (US)

(73) Assignee: NATURAL EXTRACTION SYSTEMS, LLC, Boulder, CO (US)

(21) Appl. No.: 18/546,085

(22) PCT Filed: Feb. 10, 2022
(86) PCT No.: PCT/US2022/016001

§ 371 Date: Aug. 10, 2023

Related U.S. Application Data
(60) Provisional application No. 63/154,512, filed on Feb. 26, 2021.
(60) Provisional application No. 63/154,545, filed on Feb. 26, 2021.
(60) Provisional application No. 63/191,817, filed on May 21, 2021.
(60) Provisional application No. 63/191,834, filed on May 21, 2021.
(60) Provisional application No. 63/191,848, filed on May 21, 2021.
(60) Provisional application No. 63/191,879, filed on May 21, 2021.
(60) Provisional application No. 63/194,814, filed on May 28, 2021.
(60) Provisional application No. 63/254,434, filed on Oct. 11, 2021.
(60) Provisional application No. 63/256,456, filed on Oct. 15, 2021.

(51) Int. Cl. A61K 9/08; A61K 31/055; A61K 31/12; A61K 31/122; A61K 31/137; A61K 31/343; A61K 31/352; A61K 31/355; A61K 31/365; A61K 31/4741; A61K 31/565; A61K 47/02; A61K 47/10

(52) U.S. Cl. CPC A61K 9/08; A61K 31/055; A61K 31/12; A61K 31/122; A61K 31/137; A61K 31/343; A61K 31/352; A61K 31/355; A61K 31/365; A61K 31/4741; A61K 31/565; A61K 47/02; A61K 47/10

(57) ABSTRACT

This patent document describes excipients that allow certain bioactive agents that exist as hydrophobic molecules to be reformulated as anions that display improved hydrophilicity, which can improve the bioavailability and pharmacokinetics of broad classes of pharmaceuticals.

### CROSS-REFERENCE TO RELATED APPLICATIONS

This patent application claims priority to U.S. Provisional Patent Application No. 63/154,512, filed Feb. 26, 2021; U.S. Provisional Patent Application No. 63/154,545, filed Feb. 26, 2021; U.S. Provisional Patent Application No. 63/191,817, filed May 21, 2021; U.S. Provisional Patent Application No. 63/191,834, filed May 21, 2021; U.S. Provisional Patent Application No. 63/191,848, filed May 21, 2021; U.S. Provisional Patent Application No. 63/191,879, filed May 21, 2021; U.S. Provisional Patent Application No. 63/194,814, filed May 28, 2021; U.S. Provisional Patent Application No. 63/254,434, filed Oct. 11, 2021; U.S. Provisional Patent Application No. 63/256,456, filed Oct. 15, 2021; and U.S. Provisional Patent Application No. 63/282,126, filed Nov. 22, 2021, each of which is incorporated by reference in its entirety.

### BACKGROUND

The “Rule of Five” states that the logarithm base-10 of the octanol-water partition coefficient of a druglike molecule is generally no greater than 5. A generally-applicable strategy to overcome this feature of the Rule of Five is desirable.

### SUMMARY

An octanol-water partition coefficient with a logarithm base-10 greater than 5 limits solubility in bodily fluids, which limits bioavailability. Many classes of hydrophobic bioactive molecules can be converted into anions that display improved octanol-water partition coefficients using simple acid/base chemistry. Such anions convert back into their parent bioactive molecules upon administration to a subject. Various aspects of this patent document relate to the discovery that the anionic form of a bioactive agent can overcome the octanol-water partition coefficient limitation set forth in the Rule of Five.

### DETAILED DESCRIPTION

Various aspects of this patent document relate to a composition, comprising a solvent, a Brønsted base, a cation, an anion, and a molecule, wherein the solvent has the chemical formula CₓH_{y}O_{z}; each carbon atom of the solvent is an unsaturated carbon atom; x is an integer of at least 2 and no greater than 7; either the solvent has the chemical formula C₆H₁₂O₆, or y equals 2× plus 2; the solvent comprises z hydroxyl group(s); each hydroxyl group of the solvent is covalently bound to a different carbon atom of the solvent; z is an integer of at least 1 and no greater than x; the Brønsted base is a conjugate base of the solvent; the Brønsted base has the chemical formula CₓH_{w}O_{z}¹⁻; the Brønsted base comprises 1 oxide group; the Brønsted base comprises z minus 1 hydroxyl group(s); w equals y minus 1; the cation is either a metal cation or an ammonium cation; the anion is a conjugate base of the molecule; the molecule has an acid dissociation constant in water of at least 50 femtomolar and no greater than 50 nanomolar for conversion of the molecule into the anion; the solvent has a solubility in water of at least 100 grams per liter; the liquid phase has a molar concentration of the solvent, which is at least 5 molar and no greater than 17 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

“Comprise” refers to an open set; a composition that comprises a solvent, a Brønsted base, a cation, an anion, and a molecule, for example, can also comprise the anionic form of a bioactive agent.

“Dissolved” refers to a chemical species that is solvated in a liquid phase, for example, by a solvent; a chemical species that is present within a phase that is dispersed within a liquid phase, such as the dispersed phase of an emulsion, is not dissolved in the liquid phase; a chemical species that is non-covalently bound to any chemical species that is a solid in the absence of a solvent, such as a cyclodextrin, is not dissolved.

“Ammonium cation” refers to either ammonium (“NH₄+”) or an aminium cation.

In some specific embodiments, the solvent has the chemical formula C₂H₆O, C₃H₈O, C₃H₈O₂, C₃H₈O₃, C₄H₁₀O, C₄H₁₀O₂, C₄H₁₀O₃, C₄H₁₀O₄, C₅H₁₂O, C₅H₁₂O₂, C₅H₁₂O₃, C₅H₁₂O₄, C₅H₁₂O₅, C₆H₁₂O₆, C₆H₁₄O, C₆H₁₄O₂, C₆H₁₄O₃, C₆H₁₄O₄, C₆H₁₄O₅, C₆H₁₄O₆, C₇H₁₆O, C₇H₁₆O₂, C₇H₁₆O₃, C₇H₁₆O₄, C₇H₁₆O₅, C₇H₁₆O₆, or C₇H₁₆O₇. In some very specific embodiments, the solvent has the chemical formula C₂H₆O, C₃H₈O₂, C₃H₈O₃, C₄H₁₀O₂, C₄H₁₀O₄, C₅H₁₂O₅, C₆H₁₂O₆, C₆H₁₄O₆, or C₇H₁₆O₇.

In some very specific embodiments, the solvent is 1,2-propanediol; 1,3-propanediol; 1,2,3-propanetriol; 1,3-butanediol; 1,4-butanediol; 2,3-butanediol; butane-1,2,3,4-tetrol; pentane-1,2,3,4,5-pentol; cyclohexane-1,2,3,4,5,6-hexol; hexane-1,2,3,4,5,6-hexol; or heptane-1,2,3,4,5,6,7-heptol.

In some specific embodiments, the liquid phase has a molar concentration of the solvent, which is at least 5 molar and no greater than 14 molar.

In some specific embodiments, the Brønsted base has the chemical formula C₂H₅O¹⁻, C₃H₇O¹⁻, C₃H₇O₂¹, C₃H₇O₃¹⁻, C₄H₉O¹⁻, C₄H₉O₂¹⁻, C₄H₉O₃¹⁻, C₄H₉O₄¹⁻, C₅H₁₁O¹⁻, C₅H₁₁O₂¹⁻, C₅H₁₁O₃¹⁻, C₅H₁₁O₄¹⁻, C₅H₁₁O₅¹⁻, C₆H₁₁O₆¹⁻, C₆H₁₃O¹⁻, C₆H₁₃O₂¹⁻, C₆H₁₃O₃¹⁻, C₆H₁₃O₄¹⁻, C₆H₁₃O₅¹⁻, C₆H₁₃O₆¹⁻, C₇H₁₅O¹⁻, C₇H₁₅O₂¹⁻, C₇H₁₅O₃¹⁻, C₇H₁₅O₄¹⁻, C₇H₁₅O₅¹⁻, C₇H₁₅O₆¹⁻, or C₇H₁₅O₇¹⁻. In some very specific embodiments, the Brønsted base has the chemical formula C₂H₅₀¹⁻, C₃H₇O₂¹⁻, C₃H₇O₃¹⁻, C₄H₉O₂¹⁻, C₄H₉O₄¹⁻, C₅H₁₁O₅¹⁻, C₆H₁₁O₆¹⁻, C₆H₁₃O₆¹⁻, or C₇H₁₅O₇¹⁻.

In some embodiments, the Brønsted base is ethoxide; propane-1-oxide; 1-hydroxypropane-2-oxide; 2-hydroxypropane-1-oxide; 3-hydroxypropane-1-oxide; 1,3-dihydroxypropane-2-oxide; 2,3-dihydroxypropane-1-oxide; butane-1-oxide; butane-2-oxide; 1-hydroxybutane-2-oxide; 2-hydroxybutane-1-oxide; 3-hydroxybutane-1-oxide; 3-hydroxybutane-2-oxide; 4-hydroxybutane-1-oxide; 4-hydroxybutane-2-oxide; 1,3,4-trihydroxybutane-2-oxide; 2,3,4-trihydroxybutane-1-oxide; pentane-1-oxide; pentane-2-oxide; pentane-3-oxide; 1,2,4,5-tetrahydroxypentane-3-oxide; 1,3,4,5-tetrahydroxypentane-2-oxide; 2,3,4,5-tetrahydroxypentane-1-oxide; 1,2,4,5,6-pentahydroxyhexane-3-oxide; 1,3,4,5,6-pentahydroxyhexane-2-oxide; 2,3,4,5,6-pentahydroxyhexane-1-oxide; 2,3,4,5,6-pentahydroxycyclohexane-1-oxide; 1,2,3,5,6,7-hexahydroxyheptane-4-oxide; 1,2,4,5,6,7-hexahydroxyheptane-3-oxide; 1,3,4,5,6,7-hexahydroxyheptane-2-oxide; or 2,3,4,5,6,7-hexahydroxyheptane-1-oxide. In some specific embodiments, the Brønsted base is ethoxide; 1-hydroxypropane-2-oxide; 2-hydroxypropane-1-oxide; 3-hydroxypropane-1-oxide; 1,3-dihydroxypropane-2-oxide; 2,3-dihydroxypropane-1-oxide; 1,3,4-trihydroxybutane-2-oxide; 2,3,4-trihydroxybutane-1-oxide; 1,2,4,5-tetrahydroxypentane-3-oxide; 1,3,4,5-tetrahydroxypentane-2-oxide; 2,3,4,5-tetrahydroxypentane-1-oxide; 1,2,4,5,6-pentahydroxyhexane-3-oxide; 1,3,4,5,6-pentahydroxyhexane-2-oxide; 2,3,4,5,6-pentahydroxyhexane-1-oxide; 2,3,4,5,6-pentahydroxycyclohexane-1-oxide; 1,2,3,5,6,7-hexahydroxyheptane-4-oxide; 1,2,4,5,6,7-hexahydroxyheptane-3-oxide; 1,3,4,5,6,7-hexahydroxyheptane-2-oxide; or 2,3,4,5,6,7-hexahydroxyheptane-1-oxide. In some very specific embodiments, the Brønsted base is 1-hydroxypropane-2-oxide; 2-hydroxypropane-1-oxide; 3-hydroxypropane-1-oxide; 1,3-dihydroxypropane-2-oxide; or 2,3-dihydroxypropane-1-oxide.

In some specific embodiments, the molar concentration of the Brønsted base that is dissolved in the liquid phase is at least 10 nanomolar and no greater than 100 millimolar.

In some specific embodiments, the molar concentration of the solvent in the liquid phase is at least 100 times greater than the molar concentration of the Brønsted base that is dissolved in the liquid phase.

In some embodiments, the composition comprises water and hydroxide; the hydroxide is a solute that is dissolved in the liquid phase; the liquid phase has a molar concentration of the hydroxide that is dissolved in the liquid phase; and the liquid phase has a molar concentration of water, which is at least 10 times and no more than 1 billion times greater than the molar concentration of the hydroxide that is a dissolved in the liquid phase. In some specific embodiments, the liquid phase has a molar concentration of water, which is at least 100 times and no more than 100 million times greater than the molar concentration of the hydroxide that is a dissolved in the liquid phase. In some very specific embodiments, the liquid phase has a molar concentration of water, which is at least 1000 times and no more than 10 million times greater than the molar concentration of the hydroxide that is a dissolved in the liquid phase.

In some embodiments, the composition comprises ethoxide.

Various aspects of this patent document relate to a composition for use as a medicament, wherein the composition comprises a salt; the salt comprises an anion and a cation; the anion is a conjugate base of a molecule; and the molecule has an acid dissociation constant in water of at least 50 femtomolar and no greater than 50 nanomolar for conversion of the molecule into the anion.

In some specific embodiments, the cation in is a metal cation. In some very specific embodiments, the cation is a metal cation, and the metal cation is lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); or copper (II) cation (“Cu++”).

In some specific embodiments, the cation is a metal cation, and the metal cation is potassium cation or sodium cation. In some very specific embodiments, the cation is a metal cation, and the metal cation is potassium cation. In some very specific embodiments, the cation is a metal cation, and the metal cation is sodium cation.

In some specific embodiments, the cation is an ammonium cation. In some very specific embodiments, the cation is an ammonium cation, and the ammonium cation is ammonium; protonated ethanolamine; choline; protonated lysine; protonated arginine; or protonated sphingosine.

In some specific embodiments, the molar concentration of the cation that is dissolved in the liquid phase is at least 100 nanomolar and no greater than 100 millimolar.

In some embodiments, the molecule has the chemical formula C_{(MC)}H_{(MH)}X_{(MX)}N_{(MN)}O_{(MO)}S_{(MS)}; the anion has the chemical formula C_{(AC)}H_{(AH)}X_{(AX)}N_{(AN)}O_{(AO)}S_{(AS)}¹⁻; MC is an integer from 6 to 32; MH is an integer from 6 to 50; MH is both at least half MC and no more than 1.75 times greater than MC; X is a halogen selected from F, Cl, Br, and I; MX is either 0, 1, or 2; MN is either 0, 1, or 2; MO is an integer from 1 to 12; MC is at least 2 times greater than MO; MS is either 0 or 1; AC equals MC; AH equals MH minus 1; AX equals MX; AN equals MN; AO equals MO; and AS equals MS. In some specific embodiments, the molecule has the chemical formula C_{(MC)}H(MH)N_{(MN)}O(MO); and the anion has the chemical formula C_{(AC)}H_{(AH)}N_{(AN)}O(AO)¹⁻. In some specific embodiments, the molecule has the chemical formula C_{(MC)}H_{(MH)}O_{(MO)}S_{(MS)}; and the anion has the chemical formula C_{(AC)}H_{(AH)}O_{(AO)}S_{(AS)}¹⁻. In some specific embodiments, the molecule has the chemical formula C_{(MC)}H_{(MH)}X_{(MX)}O_{(MO)}; and the anion has the chemical formula C_{(AC)}H_{(AH)}X_{(AX)}O_{(AO)}¹⁻. In some very specific embodiments, the molecule has the chemical formula C_{(MC)}H_{(MH)}O_{(MO)}; and the anion has the chemical formula C_{(AC)}H_{(AH)}O_{(AO)}¹⁻.

The nature of the molecule and the anion is not particularly limiting so long as the molecule has an acid dissociation constant in water of at least 50 femtomolar and no greater than 50 nanomolar for conversion of the molecule into the anion. A molecule that comprises a hydroxyl group that is bound to an unsaturated carbon atom typically has an acid dissociation constant in water of at least 50 femtomolar and no greater than 50 nanomolar for conversion of the molecule into the anion provided that the hydroxyl group is the most acidic functional group of the molecule. Examples of such molecules include numerous natural products such as eugenol, thymol, and carvacrol.

In some embodiments, the molecule is selected from acacetin; actiphenol; albofungin; alizarin; alizarin 1-methyl ether; alizarin 2-methyl ether; alkannin; aloe emodin; alpha-estradiol; alpha-kosin; alpha-naphtholphthalein; alpha-peltatin; alpha-thujaplicin; alpha-tocopherol; alpha-tocotrienol; alpha-zearalenol; ampelopsin; anthragallol; anthralin; anthranol; anthrapurpurin; anthrarobin; anthrarufin; apigenin; apocynin; arbutamine; aspidinol; atovaquone; atranorin; aza-[6]-gingerol; baicalein; baptigenin; benzarone; benzbromarone; benzestrol; benziodarone; benzophenone-6; benzoresorcinol; beta-kosin; beta-mangostin; beta-peltatin; beta-resorcylaldehyde; beta-thujaplicin; beta-tocopherol; beta-tocotrienol; bifluranol; biochanin A; bisphenol A; bisphenol B; brazilin; brodifacoum; bromadiolone; bromosalicylchloranilide; bromosaligenin; buparvaquone; butopyronoxyl; butylated hydroxyanisole; butylated hydroxytoluene; butylparaben; cabenegrin A-I; cabenegrin A-II; cannabidiol; cannabidiphorol; cannabidivarin; cannabinol; capsaicin; carvacrol; catechin; cavicularin; chavicol; chlorindanol; chrysarobin; chrysin; clofoctol; clorophene; collinomycin; combretastatin; combretastatin A-1; combretastatin A-4; combretastatin B-1; coniferyl alcohol; cotoin; coumafuryl; coumestrol; curcumin; curvularin; cyclovalone; daidzein; danthron; daphnetin; datiscetin; DBMC; deferiprone; dehydroequol; delta8-tetrahydrocannabinol; delta-tocopherol; dexanabinol; dichlorophen; dicoumarol; dienestrol; diethylstilbestrol; diethylstilbestrol monobenzyl ether; difenacoum; dihydrocapsaicin; dihydrokaempferide; dihydrokaempferol; dihydroresveratrol; dimethylheptyl cannabidiol; dimethylstilbestrol; dioxybenzone; dobutamine; embelin; emodin; enterolactone; epicocconone; epigallocatechin gallate; equilenin; equilin; equol; ergoflavin; eriodictyol; esculetin; estradiol; estradiol 170-cyclopentanepropanoate; estradiol enanthate; estradiol undecylate; estradiol valerate; estriol; estrone; eta-tocopherol; ethinyl estradiol; ethyl biscoumacetate; ethyl maltol; ethyl vanillin; ethylidene dicoumarol; ethylparaben; eugenol; euparin; eupatorin; fenoterol; fenretinide; fisetin; flavopurpurin; flocoumafen; flopropione; fluorescein; formestane; formononetin; fraxetin; fulvestrant; fumigatin; fusarubin; fuscin; fustin; galangin; gallein; gallocatechin; gamma-mangostin; gamma-thujaplicin; gamma-tocopherol; gardenin A; gardenin B; gardenin C; gardenin D; gardenin E; genistein; gentisin; gentisyl alcohol; geranylhydroquinone; gossypol; guaiacol; hematein; hematoxylin; heminordihydroguaiaretic acid; herqueinone; hesperetin; hexestrol; homocapsaicin I; homocapsaicin II; homodihydrocapsaicin; homoeriodictyol; homosalate; homovanillyl alcohol; honokiol; hydrocarbostyril; hydroxytyrosol; hymecromone; ilimaquinone; irigenin; irisolone; isoestradiol; isoeugenol; isomaltol; isosilybin A; ivacaftor; javanicin; juglone; kaempferol; lapachol; lawsone; leucocyanidin; licochalcone A; licochalcone B; licochalcone C; licochalcone D; licochalcone E; licochalcone F; linomide; luteolin; magnolol; maltol; mangostin; medicagol; medicarpin; menadiol; mestilbol; meta-cresol; methestrol; methylchrysin; methylparaben; mexenone; monobenzone; morin; morphenol; moxestrol; myricetin; nabilone; naphthoresorcinol; naringenin; nimbiol; nonivamide; nordihydrocapsaicin; nordihydroguaiaretic acid; octabenzone; oleocanthal; oroxylin A; ortho-benzylphenol; orthocaine; ortho-cresol; oryzanol A; oryzanol C; osajin; osalmid; ostruthin; ourateacatechin; oxabolone; oxabolone 17-cyclopentanepropionate; oxybenzone; oxymesterone; oxymetholone; oxyphenbutazone; para-anol; para-benzylphenol; para-cresol; para-tert-pentylphenol; para-vinylguaiacol; parietin; paroxypropione; parvaquone; pectolinarigenin; perezone; phaseolin; phenolphthalol; phenprocoumon; phloretin; phthiocol; pinosylvin; pinosylvin monomethyl ether; plumbagin; pratensein; probucol; procerin; propofol; propyl gallate; propylparaben; protiofate; protocatechualdehyde; prunetin; pseudobaptigenin; psiguadial A; psiguadial B; psiguadial C; psiguadial D; psoralidin; purpurin; purpurin 1-methyl ether; purpurin 2,4-dimethyl ether; purpurin 2-methyl ether; purpurogallin; quercetagetin; quercetin; quinalizarin; raspberry ketone; resacetophenone; resveratrol; rhamnetin; rottlerin; rottlerin 5,7-dimethyl ether; rubiadin; salicyl alcohol; salicylaldehyde; salicylanilide; scopoletin; scutellarein; siccanin; silybin A; sparassol; stilbestrol; strobilurin F; sulfuretin; sumatrol; synhexyl; syringaldehyde; taxodione; tectorigenin; tetrahydrocannabinol; tetrahydrocannabinol-C4; tetrahydrocannabiorcol; tetrahydrocannabiphorol; tetrahydrocannabivarin; tetrahydrorottlerin; thymol; tioclomarol; tolcapone; trilostane; tyrosol; uliginosin A; uliginosin B; umbelliferone; vanillin; vanillyl alcohol; violacein; viridicatin; xanthoxylin; xibornol; zearalenone; zeranol; zeta2-tocopherol; zingerone; [1]-gingerol; [10]-gingerol; [12]-gingerol; [14]-gingerol; [16]-gingerol; [2]-gingerol; [3]-gingerol; [4]-gingerol; [5]-gingerol; [6]-gingerol; [7]-gingerol; [8]-gingerol; [9]-gingerol; 16-epiestriol; 17alpha-dihydroequilin; 2,5-di-tert-pentylhydroquinone; 2′,7′-dichlorofluorescein; 2-naphthol; 3,6-dimethylmangostin; 3-methoxy-4-hydroxyphenylglycol; 4′,5′-dichlorofluorescein; 4′,5′-diiodofluorescein; 4′-fluorocannabidiol; 4-hexylresorcinol; 4-O-methylhonokiol; 4′-O-methylkaempferol; 5-ethoxy-10-gingerol; 5-O-methyl-9-gingerol; 6-deoxy-gamma mangostin; 8-isoestrone; 9-hydroxy-[6]-gingerol; abediterol; abnormal cannabidiol; acerosin; acetosyringone; adipostatin A; afzelechin; aleuritin; alnetin; alternariol; AM-087; AM-1714; AM-2389; AM-4030; AM404; AM-411; AM-855; AM-905; AM-919; AM-938; amentoflavone; AMG-1; AMG-36; AMG-41; amylmetacresol; apiforol; arctigenin; artocarpetin; artocarpin; ascofuranone; aureusidin; axillarin; azaleatin; bakuchiol; balanophonin; benzophenone-2; bilobol; bisdemethoxycurcumin; bisphenol F; blumeatin; butin; caflanone; calphostin A; calphostin B; calphostin C; calphostin D; calycosin; campesteryl ferulate; canbisol; cannabichromene; cannabicyclohexanol; cannabicyclol; cannabicyclovarin; cannabigerol; cannabigerovarin; cannflavin A; cannflavin B; cannflavin C; canolol; chaetochromin A; chaetochromin C; chaetochromin D; chavibetol; chrysoeriol; cirsilineol; cirsiliol; cirsimaritin; corymbosin; coumatetralyl; CP 55,244; CP 55,940; damnacanthal; DB-2073; debromomarinone; delta-tocotrienol; demethoxycurcumin; derrubone; dianol; dihydrokanakugiol; dihydromorin; dimethylheptylpyran; diosmetin; DOPAL; DOPEG; drupanol; durantin A; echioidinin; enterodiol; epicatechin gallate; ermanin; estetrol; eupatilin; euxanthone; ferruginol; ferujol; fisetinidol; gamma-tocotrienol; garbanzol; genkwanin; geraldone; geranin A; glabrene; glabridin; glyceollin I; glyceollin II; glyceollin III; glyceollin IV; glycinol; glycitein; glycyrrhizol A; gossypetin; guibourtinidol; heptylparaben; hispidulin; HU-210; HU-243; HU-331; hydroxymatairesinol; hymenoxin; hypolaetin; iodoresiniferatoxin; irilone; isojacareubin; isoliquiritigenin; isorhamnetin; isosakuranetin; isoscutellarein; isosilychristin; isoxanthohumol; jaceosidin; kanakugiol; KM-233; kuwanon G; lariciresinol; laricitrin; leptosidin; leptosphaerin A; leucofisetinidin; leucopelargonidin; leucopeonidin; liquiritigenin; luteoforol; luteone; macelignan; matairesinol; mearnsetin; meciadanol; melacacidin; mequinol; mesquitol; mikanin; mutisianthol; myricanone; naphthazarin; negletein; nepetin; nevadensin; nigrosporin B; nodifloretin; norartocarpetin; norwogonin; O-1602; O-1656; O-1660; O-1871; O-806; obovatol; olivetol; onopordin; oritin; orobol; ortho-phenylphenol; oxyanthrarufin; oxychrysazin; pachypodol; patuletin; pedalitin; perrottetinene; phenylacetylrinvanil; phylloquinol; piceatannol; pilloin; pinobanksin; pinocembrin; pinoresinol; pinostrobin; plicadin; pomiferin; poriol; pratol; primetin; primuletin; PSB-SB-487; pseudoisoeugenol; psi-tectorigenin; pterostilbene; purpuroxanthin; quinizarin; resiniferatoxin; retusin; rhamnazin; robinetinidol; sakuranetin; sappanol; scaposin; scillavone A; scillavone B; secoisolariciresinol; selligueain A; serpyllin; sesamol; shogaol; silychristin; silydianin; sinapyl alcohol; sitostanyl ferulate; sitosteryl ferulate; sophoradin; sophoraflavanone G; sorbifolin; spinacetin; spinochrome B; sterubin; stigmasteryl ferulate; stigmatellin; sudachitin; syringaresinol; syringetin; syringol; taxifolin; taxodone; techtochrysin; thunberginol F; thunberginol G; tinabinol; tinyatoxin; tithonine; tricetin; tricin; ubiquinol; urolithin A; urolithin B; velutin; verrulactone A; verrulactone B; verrulactone E; wedelolactone; wighteone; wightin; wogonin; xanthohumol; xanthomicrol; xylindein; zapotinin; (C6)-CP 47,497; (C7)-CP 47,497; (C9)-CP 47,497; [10]-dehydroshogaol; [10]-shogaol; [12]-shogaol; [4]-shogaol; [8]-dehydroshogaol; [8]-shogaol; 1,6-dihydroxyanthraquinone; 1,7-bis(4-hydroxyphenyl)-1,4,6-heptatrien-3-one; 11-hydroxytetrahydrocannabinol; 11-hydroxyyangonin; 11-methoxy-12-hydroxydehydrokavain; 11-nor-9beta-hydroxyhexahydrocannabinol; 3′,5′-di-tert-butyl-4′,7-dihydroxyflavone; 3,5,7-trihydroxy-8-isoprenyl-2-(4-methoxyphenyl)-4H-chromen-4-one; 3-hydroxychrysazin; 4′,7-dihydroxyflavone; 4-hydroxycoumarin; 4-hydroxyphenylacetaldehyde; 5-chloro-2-hydroxybenzophenone; 5-hydroxykavain; 5-O-methylgenistein; 6-hydroxyflavone; 6-hydroxyluteolin; 7,8-dihydroxyflavone; 7-O-methylisoxanthohumol; 7-O-n-pentylisoxanthohumol; 9-hydroxychrysarobin; sesaminol; omeprazole; and psilocin.

In some specific embodiments, the molecule has an acid dissociation constant in water of at least 100 femtomolar and no greater than 10 nanomolar for conversion of the molecule into the anion. In some very specific embodiments, the molecule has an acid dissociation constant in water of at least 5 picomolar and no greater than 5 nanomolar for conversion of the molecule into the anion.

In all embodiments, the molecule and anion buffer the pH of the liquid phase such that the bioactive agent exists primarily in an anionic form. In some very specific preferred embodiments, the molecule is the molecular form of the bioactive agent and the anion is the anionic form of the bioactive agent, for example, such that the anionic and molecular forms of the bioactive agent act as their own buffer. In such embodiments, the anionic form of the bioactive agent is typically present at a therapeutically effective concentration, and the molecular form of the bioactive agent is typically present at a concentration that is less than any known therapeutically effective concentrations for the molecular form.

In some specific embodiments, the molar concentration of the anion that is dissolved in the liquid phase is at least 100 nanomolar and no greater than 100 millimolar.

Various aspects of this patent document relate to a composition described anywhere in this patent document for use as a medicament.

In some embodiments, the composition is formulated for administration to a human; and the composition is formulated to convert the anion into the molecule ex vivo prior to the administration.

In some embodiments, the composition is formulated for administration to a human; and the composition is formulated to convert the anion into the molecule in situ subsequent to the administration.

Various aspects of this patent document relate to a method to administer a bioactive agent to a subject, comprising providing a composition described anywhere in this patent document, and administering the composition to the subject.

In some embodiments, the administering is oral, sublingual, sublabial, buccal, rectal, intranasal, inhalational, transmucosal, topical, transdermal, intravenous, intramuscular, subcutaneous, intradermal, intraocular, parenteral, intrathecal, intralesional, or intratumoral administering. In some specific embodiments, the administering is oral, sublingual, or buccal administering. In some specific embodiments, the administering is topical or transdermal administering.

In some specific embodiments, the composition is formulated to allow the conversion of an anionic form of the bioactive agent into a molecular form of the bioactive agent. In some very specific embodiments, the composition is formulated to allow the conversion of the anionic form of the bioactive agent into the molecular form of the bioactive agent ex vivo prior to administering the composition to the subject. In some very specific embodiments, the composition is formulated to allow the conversion of the anionic form of the bioactive agent into the molecular form of the bioactive agent in situ subsequent to administering the composition to the subject.

In some specific embodiments, administering comprises topical administration. In some very specific embodiments, the composition is formulated for topical administration, and topical administration results in conversion of an anionic form of the bioactive agent into a molecular form of the bioactive agent.

In some specific embodiments, administering comprises oral administration. In some very specific preferred embodiments, the composition is formulated for oral administration, and the composition is formulated to allow the conversion of an anionic form of the bioactive agent into a molecular form of the bioactive agent before the bioactive agent reaches the stomach of the subject to allow absorption of the bioactive agent by the epithelial lining of the gastrointestinal tract between the lips and the stomach, excluding the stomach and the outer surfaces of the lips, and including the esophagus and the inner surfaces of the lips.

In some specific embodiments, the subject is a rodent, lagomorph, feline, canine, porcine, ovine, caprine, lama, vicugna, bovine, equine, or primate. In some very specific embodiments, the subject is human.

What is claimed is:

1. A composition, comprising a solvent, a Brønsted base, a cation, an anion, a molecule, water, and hydroxide, wherein: the solvent has the chemical formula CₓH_{y}O_{z}, wherein each carbon atom of the solvent is an unsaturated carbon atom; x is an integer of at least 2 and no greater than 7; either the solvent has the chemical formula C₆H₁₂O₆, or y equals 2× plus 2; the solvent comprises z hydroxyl group(s); each hydroxyl group of the solvent is covalently bound to a different carbon atom of the solvent; z is an integer of at least 1 and no greater than x; the Brønsted base is a conjugate base of the solvent; the Brønsted base has the chemical formula CₓH_{w}O_{z}¹⁻, wherein the Brønsted base comprises 1 oxide group; the Brønsted base comprises z minus 1 hydroxyl group(s); and w equals y minus 1; the cation is either a metal cation or an ammonium cation; the anion is a conjugate base of the molecule; the molecule has an acid dissociation constant in water of at least 50 femtomolar and no greater than 50 nanomolar for conversion of the molecule into the anion; the solvent has a solubility in water of at least 100 grams per liter; the liquid phase has a molar concentration of the solvent, which is at least 5 molar and no greater than 17 molar; the Brønsted base, the cation, the anion, the molecule, and the hydroxide are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; the liquid phase has a molar concentration of the molecule that is dissolved in the liquid phase, which is less than the molar concentration of the anion that is dissolved in the liquid phase; the liquid phase has a molar concentration of the hydroxide that is dissolved in the liquid phase; and the liquid phase has a molar concentration of water, which is at least 10 times and no more than 1 billion times greater than the molar concentration of the hydroxide that is a dissolved in the liquid phase.

2. The composition of claim 1, wherein the molecule has the chemical formula C₂H₆O, C₃H₈O₂, C₃H₈O₃, C₄H₁₀O₂, C₄H₁₀O₄, C₅H₁₂O₅, C₆H₁₂O₆, C₆H₁₄O₆, or C₇H₁₆O₇.

3. The composition of claim 1 or 2, wherein the molecule is 1,2-propanediol; 1,3-propanediol; 1,2,3-propanetriol; 1,3-butanediol; 1,4-butanediol; 2,3-butanediol; butane-1,2,3,4-tetrol; pentane-1,2,3,4,5-pentol; cyclohexane-1,2,3,4,5,6-hexol; hexane-1,2,3,4,5,6-hexol; or heptane-1,2,3,4,5,6,7-heptol.

4. The composition of any one of claims 1-3, wherein the Brønsted base is 1-hydroxypropane-2-oxide; 2-hydroxypropane-1-oxide; 3-hydroxypropane-1-oxide; 1,3-dihydroxypropane-2-oxide; or 2,3-dihydroxypropane-1-oxide.

5. A composition, comprising a solvent, a Brønsted base, a cation, an anion, and a molecule, wherein: the solvent is 1,2-propanediol; the Brønsted base is 1-hydroxypropane-2-oxide or 2-hydroxypropane-1-oxide; the anion is a conjugate base of the molecule; the molecule has an acid dissociation constant in water of at least 50 femtomolar and no greater than 50 nanomolar for conversion of the molecule into the anion; the liquid phase has a molar concentration of the solvent, which is at least 5 molar and no greater than 14 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

6. A composition, comprising a solvent, a Brønsted base, a cation, an anion, and a molecule, wherein: the solvent is 1,3-propanediol; the Brønsted base is 3-hydroxypropane-1-oxide; the anion is a conjugate base of the molecule; the molecule has an acid dissociation constant in water of at least 50 femtomolar and no greater than 50 nanomolar for conversion of the molecule into the anion; the liquid phase has a molar concentration of the solvent, which is at least 5 molar and no greater than 14 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

7. A composition, comprising a solvent, a Brønsted base, a cation, an anion, and a molecule, wherein: the solvent is 1,2,3-propanetriol; the Brønsted base is 1,3-dihydroxypropane-2-oxide or 2,3-dihydroxypropane-1-oxide; the anion is a conjugate base of the molecule; the molecule has an acid dissociation constant in water of at least 50 femtomolar and no greater than 50 nanomolar for conversion of the molecule into the anion; the liquid phase has a molar concentration of the solvent, which is at least 5 molar and no greater than 14 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

8. A composition, comprising a solvent, a Brønsted base, a cation, an anion, and a molecule, wherein: the solvent is butane-1,2,3,4-tetrol; the Brønsted base is 1,3,4-trihydroxybutane-2-oxide or 2,3,4-trihydroxybutane-1-oxide; the anion is a conjugate base of the molecule; the molecule has an acid dissociation constant in water of at least 50 femtomolar and no greater than 50 nanomolar for conversion of the molecule into the anion; the liquid phase has a molar concentration of the solvent, which is at least 5 molar and no greater than 14 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

9. A composition, comprising a solvent, a Brønsted base, a cation, an anion, and a molecule, wherein: the solvent is pentane-1,2,3,4,5-pentol; the Brønsted base is 1,2,4,5-tetrahydroxypentane-3-oxide, 1,3,4,5-tetrahydroxypentane-2-oxide, or 2,3,4,5-tetrahydroxypentane-1-oxide; the anion is a conjugate base of the molecule; the molecule has an acid dissociation constant in water of at least 50 femtomolar and no greater than 50 nanomolar for conversion of the molecule into the anion; the liquid phase has a molar concentration of the solvent, which is at least 5 molar and no greater than 14 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

10. A composition, comprising a solvent, a Brønsted base, a cation, an anion, and a molecule, wherein: the solvent is cyclohexane-1,2,3,4,5,6-hexol; the Brønsted base is 2,3,4,5,6-pentahydroxycyclohexane-1-oxide; the anion is a conjugate base of the molecule; the molecule has an acid dissociation constant in water of at least 50 femtomolar and no greater than 50 nanomolar for conversion of the molecule into the anion; the liquid phase has a molar concentration of the solvent, which is at least 5 molar and no greater than 14 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

11. A composition, comprising a solvent, a Brønsted base, a cation, an anion, and a molecule, wherein: the solvent is hexane-1,2,3,4,5,6-hexol; the Brønsted base is 1,2,4,5,6-pentahydroxyhexane-3-oxide, 1,3,4,5,6-pentahydroxyhexane-2-oxide, or 2,3,4,5,6-pentahydroxyhexane-1-oxide; the anion is a conjugate base of the molecule; the molecule has an acid dissociation constant in water of at least 50 femtomolar and no greater than 50 nanomolar for conversion of the molecule into the anion; the liquid phase has a molar concentration of the solvent, which is at least 5 molar and no greater than 14 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

12. A composition, comprising a solvent, a Brønsted base, a cation, an anion, and a molecule, wherein: the solvent is heptane-1,2,3,4,5,6,7-heptol; the Brønsted base is 1,2,3,5,6,7-hexahydroxyheptane-4-oxide, 1,2,4,5,6,7-hexahydroxyheptane-3-oxide, 1,3,4,5,6,7-hexahydroxyheptane-2-oxide, or 2,3,4,5,6,7-hexahydroxyheptane-1-oxide; the anion is a conjugate base of the molecule; the molecule has an acid dissociation constant in water of at least 50 femtomolar and no greater than 50 nanomolar for conversion of the molecule into the anion; the liquid phase has a molar concentration of the solvent, which is at least 5 molar and no greater than 14 molar; the Brønsted base, the cation, the anion, and the molecule are solutes that are dissolved in the liquid phase; the liquid phase has a molar concentration of the Brønsted base that is dissolved in the liquid phase, which is at least 1 nanomolar and no greater than 1 molar; the molar concentration of the solvent in the liquid phase is at least 10 times greater than the molar concentration of the Brønsted base that is dissolved in the liquid phase; the liquid phase has a molar concentration of the cation that is dissolved in the liquid phase, which is at least 10 nanomolar and no greater than 1 molar; the liquid phase has a molar concentration of the anion that is dissolved in the liquid phase, which is at least 10 nanomolar and no greater than 1 molar; and the liquid phase has a molar concentration of the molecule that is dissolved in the liquid phase, which is less than the molar concentration of the anion that is dissolved in the liquid phase.

13. The composition of any one of claims 5-12, comprising water and hydroxide, wherein the hydroxide is a solute that is dissolved in the liquid phase; the liquid phase has a molar concentration of the hydroxide that is dissolved in the liquid phase; and the liquid phase has a molar concentration of water, which is at least 10 times and no more than 1 billion times greater than the molar concentration of the hydroxide that is a dissolved in the liquid phase.

14. The composition of any one of claims 1-13, comprising ethoxide.

15. A composition for use as a medicament, wherein the composition comprises a salt; the salt comprises an anion and a cation; the anion is a conjugate base of a molecule; and the molecule has an acid dissociation constant in water of at least 50 femtomolar and no greater than 50 nanomolar for conversion of the molecule into the anion.

16. The composition of any one of claims 1-15, wherein the cation is lithium cation (“Li+”); sodium cation (“Na+”); potassium cation (“K+”); magnesium cation (“Mg++”); calcium cation (“Ca++”); zinc cation (“Zn++”); manganese cation (“Mn++”); iron (II) cation (“Fe++”); iron (III) cation (“Fe+++”); copper (I) cation (“Cu+”); copper (II) cation (“Cu++”); ammonium (“NH₄+”); protonated ethanolamine; choline; protonated lysine; protonated arginine; or protonated sphingosine.

17. The composition of any one of claims 1-16, wherein the cation is sodium cation.

18. The composition of any one of claims 1-16, wherein the cation is potassium cation.

19. The composition of any one of claims 1-18, wherein the molecule has the chemical formula C_{(MC)}H_{(MH)}X_{(MX)}N_{(MN)}O_{(MO)}S_{(MS)}; the anion has the chemical formula C_{(AC)}H_{(AH)}X_{(AX)}N_{(AN)}O_{(AO)}S_{(AS)}¹⁻; MC is an integer from 6 to 32; MH is an integer from 6 to 50; MH is both at least half MC and no more than 1.75 times greater than MC; X is a halogen selected from F, Cl, Br, and I; MX is either 0, 1, or 2; MN is either 0, 1, or 2; MO is an integer from 1 to 12; MC is at least 2 times greater than MO; MS is either 0 or 1; AC equals MC; AH equals MH minus 1; AX equals MX; AN equals MN; AO equals MO; and AS equals MS.

20. The composition of any one of claims 1-19, wherein the molecule is acacetin, which has CAS Registry Number 480-44-4 and is described in The Merck Index monograph M1284; and the anion is selected from 5-hydroxy-4-oxo-2-(4-methoxyphenyl)-4H-chromene-7-oxide and 7-hydroxy-4-oxo-2-(4-methoxyphenyl)-4H-chromene-5-oxide.

21. The composition of any one of claims 1-19, wherein the molecule is actiphenol, which has CAS Registry Number 526-02-3 and is described in The Merck Index monograph M1003; and the anion is 4,6-dimethyl-2-[1-oxo-2-(2,6-dioxopiperidin-4-yl)ethyl]phenolate.

22. The composition of any one of claims 1-19, wherein the molecule is albofungin, which has CAS Registry Number 37895-35-5 and is described in The Merck Index monograph M1476; and the anion is selected from 6-amino-21,28-dihydroxy-24-methoxy-7-methyl-5,19-dioxo-14,16,26-trioxa-6-azaheptacyclo[15.11.1.0^{2,11}.0^{4,9}.0^{13,29}.0^{18,27}.0^{20,25}]nonacosa-1(28),2(11),3,7,9,17(29),18(27),20(25)-octaene-3-oxide and 6-amino-3,21-dihydroxy-24-methoxy-7-methyl-5,19-dioxo-14,16,26-trioxa-6-azaheptacyclo[15.11.1.0^{2,11}.0^{4,9}.0^{13,29}.0^{18,27}.0^{20,25}]nonacosa-1(28),2(11),3,7,9,17(29),18(27),20(25)-octaene-28-oxide.

23. The composition of any one of claims 1-19, wherein the molecule is alizarin, which has CAS Registry Number 72-48-0 and is described in The Merck Index monograph M1513; and the anion is selected from 1-hydroxy-9,10-dioxoanthracene-2-oxide and 2-hydroxy-9,10-dioxoanthracene-1-oxide.

24. The composition of any one of claims 1-19, wherein the molecule is alizarin 1-methyl ether, which has CAS Registry Number 6170-06-5 and is described in The Merck Index monograph M1513; and the anion is 1-methoxy-9,10-dioxoanthracene-2-oxide.

25. The composition of any one of claims 1-19, wherein the molecule is alizarin 2-methyl ether, which has CAS Registry Number 6003-11-8 and is described in The Merck Index monograph M1513; and the anion is 2-methoxy-9,10-dioxoanthracene-1-oxide.

26. The composition of any one of claims 1-19, wherein the molecule is alkannin, which has CAS Registry Number 517-88-4 and is described in The Merck Index monograph M1519; and the anion is selected from 4-hydroxy-5,8-dioxo-7-(1-hydroxy-4-methylpent-3-enyl)naphthalene-1-oxide and 4-hydroxy-5,8-dioxo-6-(1-hydroxy-4-methylpent-3-enyl)naphthalene-1-oxide.

27. The composition of any one of claims 1-19, wherein the molecule is aloe emodin, which has CAS Registry Number 481-72-1 and is described in The Merck Index monograph M1570; and the anion is selected from 8-hydroxy-3-(hydroxymethyl)-9,10-dioxoanthracene-1-oxide and 8-hydroxy-6-(hydroxymethyl)-9,10-dioxoanthracene-1-oxide.

28. The composition of any one of claims 1-19, wherein the molecule is alpha-estradiol, which has CAS Registry Number 57-91-0 and is described in The Merck Index monograph M5029; and the anion is 17-hydroxy-13-methyl-6,7,8,9,11,12,14,15,16,17-decahydrocyclopenta[a]phenanthrene-3-oxide.

29. The composition of any one of claims 1-19, wherein the molecule is alpha-kosin, which has CAS Registry Number 568-50-3 and is described in The Merck Index monograph M6640; and the anion is selected from 5-hydroxy-3-methoxy-4-methyl-2-(2-methyl-1-oxopropyl)-6-{[2,6-dihydroxy-4-methoxy-5-methyl-3-(2-methyl-1-oxopropyl)phenyl]methyl}phenolate and 3-hydroxy-5-methoxy-6-methyl-4-(2-methyl-1-oxopropyl)-2-{[2,6-dihydroxy-4-methoxy-5-methyl-3-(2-methyl-1-oxopropyl)phenyl]methyl}phenolate.

30. The composition of any one of claims 1-19, wherein the molecule is alpha-naphtholphthalein, which has CAS Registry Number 596-01-0 and is described in The Merck Index monograph M7744; and the anion is 4-[3-oxo-1-(4-hydroxynaphthalen-1-yl)-2-benzofuran-1-yl]napthalene-1-oxide.

31. The composition of any one of claims 1-19, wherein the molecule is alpha-peltatin, which has CAS Registry Number 568-53-6 and is described in The Merck Index monograph M8454; and the anion is selected from 8-oxo-9-(4-hydroxy-3,5-dimethoxyphenyl)-5a,6,8a,9-tetrahydro-5H-[2]benzofuro[5,6-f][1,3]benzodioxol-4-oxide and 2,6-dimethoxy-4-(4-hydroxy-8-oxo-5a,6,8a,9-tetrahydro-5H-[2]benzofuro[5,6-f][1,3]benzodioxol-9-yl)phenolate.

32. The composition of any one of claims 1-19, wherein the molecule is alpha-thujaplicin, which has CAS Registry Number 1946-74-3 and is related to The Merck Index monograph M10814; and the anion is 2-isopropyl-7-oxocyclohepta-1,3,5-triene-1-oxide.

33. The composition of any one of claims 1-19, wherein the molecule is alpha-tocopherol, which has CAS Registry Number 59-02-9 and is described in The Merck Index monograph M10923; and the anion is 2,5,7,8-tetramethyl-2-(4,8,12-trimethyltridecyl)-3,4-dihydro-2H-chromene-6-oxide.

34. The composition of any one of claims 1-19, wherein the molecule is alpha-tocotrienol, which has CAS Registry Number 58864-81-6 and is described in The Merck Index monograph M10928; and the anion is 2,5,7,8-tetramethyl-2-(4,8,12-trimethyltrideca-3,7,11-trieneyl)-3,4-dihydro-2H-chromene-6-oxide.

35. The composition of any one of claims 1-19, wherein the molecule is alpha-zearalenol, which has CAS Registry Number 36455-71-7 and is related to The Merck Index monograph M11589; and the anion is selected from 8,18-dihydroxy-4-methyl-2-oxo-3-oxabicyclo[12.4.0]octadeca-12,14(15),16,18-tetraene-16-oxide and 8,16-dihydroxy-4-methyl-2-oxo-3-oxabicyclo[12.4.0]octadeca-12,14(15),16,18-tetraene-18-oxide.

36. The composition of any one of claims 1-19, wherein the molecule is ampelopsin, which has CAS Registry Number 27200-12-0 and is described in The Merck Index monograph M1846; and the anion is selected from 3,7-dihydroxy-4-oxo-2-(3,4,5-trihydroxyphenyl)-2,3-dihydro-4H-chromene-5-oxide; 3,5-dihydroxy-4-oxo-2-(3,4,5-trihydroxyphenyl)-2,3-dihydro-4H-chromene-7-oxide; 2,6-dihydroxy-4-(3,5,7-trihydroxy-4-oxo-2,3-dihydro-4H-chromen-2-yl)phenolate; and 2,3-dihydroxy-5-(3,5,7-trihydroxy-4-oxo-2,3-dihydro-4H-chromen-2-yl)phenolate.

37. The composition of any one of claims 1-19, wherein the molecule is anthragallol, which has CAS Registry Number 602-64-2 and is described in The Merck Index monograph M1945; and the anion is selected from 1,3-dihydroxy-9,10-dioxoanthracene-2-oxide; 2,3-dihydroxy-9,10-dioxoanthracene-1-oxide; and 3,4-dihydroxy-9,10-dioxoanthracene-2-oxide.

38. The composition of any one of claims 1-19, wherein the molecule is anthralin, which has CAS Registry Number 1143-38-0 and is described in The Merck Index monograph M1946; and the anion is 8-hydroxy-9-oxo-9,10-dihydroanthracene-1-oxide.

39. The composition of any one of claims 1-19, wherein the molecule is anthranol, which has CAS Registry Number 529-86-2 and is described in The Merck Index monograph M1948; and the anion is anthracene-9-oxide.

40. The composition of any one of claims 1-19, wherein the molecule is anthrapurpurin, which has CAS Registry Number 602-65-3 and is related to The Merck Index monograph M9326; and the anion is selected from 2,7-dihydroxy-9,10-dioxoanthracene-1-oxide; 1,7-dihydroxy-9,10-dioxoanthracene-2-oxide; and 7,8-dihydroxy-9,10-dioxoanthracene-2-oxide.

41. The composition of any one of claims 1-19, wherein the molecule is anthrarobin, which has CAS Registry Number 577-33-3 and is described in The Merck Index monograph M1011; and the anion is selected from 1,10-dihydroxy-anthracene-2-oxide; 2,10-dihydroxy-anthracene-1-oxide; and 1,2-dihydroxy-anthracene-10-oxide.

42. The composition of any one of claims 1-19, wherein the molecule is anthrarufin, which has CAS Registry Number 117-12-4 and is described in The Merck Index monograph M1950; and the anion is 5-hydroxy-9,10-dioxoanthracene-1-oxide.

43. The composition of any one of claims 1-19, wherein the molecule is apigenin, which has CAS Registry Number 520-36-5 and is described in The Merck Index monograph M1987; and the anion is selected from 7-hydroxy-4-oxo-2-(4-hydroxyphenyl)-4H-chromene-5-oxide; 5-hydroxy-4-oxo-2-(4-hydroxyphenyl)-4H-chromene-7-oxide; and 4-(5,7-dihydroxy-4-oxo-4H-chromen-2-yl)phenolate.

44. The composition of any one of claims 1-19, wherein the molecule is apocynin, which has CAS Registry Number 498-02-2 and is described in The Merck Index monograph M1998; and the anion is 4-acetyl-2-methoxyphenolate.

45. The composition of any one of claims 1-19, wherein the molecule is arbutamine, which has CAS Registry Number 128470-16-6 and is described in The Merck Index monograph M2032; and the anion is selected from 2-hydroxy-4-[1-hydroxy-2-{N-[4-(4-hydroxyphenyl)butyl]amino}ethyl]phenolate; 2-hydroxy-5-[1-hydroxy-2-{N-[4-(4-hydroxyphenyl)butyl]amino}ethyl]phenolate; and (4-{N-[2-hydroxy-2-(3,4-dihydroxyphenyl)ethyl]amino}butyl)phenolate.

46. The composition of any one of claims 1-19, wherein the molecule is aspidinol, which has CAS Registry Number 519-40-4 and is described in The Merck Index monograph M2107; and the anion is selected from 3-hydroxy-4-methyl-5-methoxy-2-(1-oxobutyl)phenolate and 3-hydroxy-6-methyl-5-methoxy-2-(1-oxobutyl)phenolate.

47. The composition of any one of claims 1-19, wherein the molecule is atovaquone, which has CAS Registry Number 95233-18-4 and is described in The Merck Index monograph M2127; and the anion is 3-[4-(4-chlorophenyl)cyclohexyl]-1,4-dioxonaphthalene-2-oxide.

48. The composition of any one of claims 1-19, wherein the molecule is atranorin, which has CAS Registry Number 479-20-9 and is described in The Merck Index monograph M2130; and the anion is selected from 2-formyl-3-hydroxy-5-methyl-4-[({3-hydroxy-2,5-dimethyl-4-[(methoxy)carbonyl]phenyl}oxy)carbonyl]phenolate; 2-formyl-3-hydroxy-5-methyl-6-[({3-hydroxy-2,5-dimethyl-4-[(methoxy)carbonyl]phenyl}oxy)carbonyl]phenolate; and 2,5-dimethyl-6-[(methoxy)carbonyl]-3-{[(3-formyl-2,4-dihydroxy-6-methylphenyl)carbonyl]oxy}phenolate.

49. The composition of any one of claims 1-19, wherein the molecule is aza-[6]-gingerol, which is related to The Merck Index monograph M5729; and the anion is 2-methoxy-4-{[N-(3-hydroxy-1-oxooctyl)amino]methyl}phenolate.

50. The composition of any one of claims 1-19, wherein the molecule is baicalein, which has CAS Registry Number 491-67-8 and is described in The Merck Index monograph M2205; and the anion is selected from 5,6-hydroxy-4-oxo-2-phenyl-4H-chromene-7-oxide; 5,7-hydroxy-4-oxo-2-phenyl-4H-chromene-6-oxide; and 6,7-hydroxy-4-oxo-2-phenyl-4H-chromene-5-oxide.

51. The composition of any one of claims 1-19, wherein the molecule is baptigenin, which has CAS Registry Number 5908-63-4 and is described in The Merck Index monograph M2223; and the anion is selected from 3-(3,4,5-trihydroxyphenyl)-4-oxo-4H-chromene-7-oxide; 5-(7-hydroxy-4-oxo-4H-chromen-3-yl)-2,3-dihydroxyphenolate; and 4-(7-hydroxy-4-oxo-4H-chromen-3-yl)-2,6-dihydroxyphenolate.

52. The composition of any one of claims 1-19, wherein the molecule is benzarone, which has CAS Registry Number 1477-19-6 and is described in The Merck Index monograph M580; and the anion is 4-[(2-ethyl-1-benzofuran-3-yl)carbonyl]phenolate.

53. The composition of any one of claims 1-19, wherein the molecule is benzbromarone, which has CAS Registry Number 3562-84-3 and is described in The Merck Index monograph M2337; and the anion is 2,6-dibromo-4-[(2-ethyl-1-benzofuran-3-yl)carbonyl]phenolate.

54. The composition of any one of claims 1-19, wherein the molecule is benzestrol, which has CAS Registry Number 85-95-0 and is described in The Merck Index monograph M69; and the anion is selected from 4-[3-ethyl-4-(4-hydroxyphenyl)hex-2-yl]phenolate and 4-[4-ethyl-5-(4-hydroxyphenyl)hex-3-yl]phenolate.

55. The composition of any one of claims 1-19, wherein the molecule is benziodarone, which has CAS Registry Number 68-90-6 and is described in The Merck Index monograph M2355; and the anion is 4-[(2-ethyl-1-benzofuran-3-yl)carbonyl]-2,6-diiodophenolate.

56. The composition of any one of claims 1-19, wherein the molecule is benzophenone-6, which has CAS Registry Number 131-54-4 and is described in The Merck Index monograph M2371; and the anion is selected from 5-methoxy-2-[(2-hydroxy-4-methoxyphenyl)carbonyl]phenolate and (2-hydroxy-4-methoxyphen-1-yl)-(6-oxo-2-methoxycyclohexa-1,3-dien-5-ylidene)methane-oxide.

57. The composition of any one of claims 1-19, wherein the molecule is benzoresorcinol, which has CAS Registry Number 131-56-6 and is described in The Merck Index monograph M2379; and the anion is selected from 3-hydroxy-4-(phenylcarbonyl)phenolate and 3-hydroxy-6-(phenylcarbonyl)phenolate.

58. The composition of any one of claims 1-19, wherein the molecule is beta-kosin, which is described in The Merck Index monograph M6640; and the anion is selected from 3,5-dihydroxy-6-methyl-2-(2-methyl-1-oxopropyl)-4-{[2-hydroxy-4,6-dimethoxy-3-methyl-5-(2-methyl-1-oxopropyl)phenyl]methyl}phenolate; 3,5-dihydroxy-4-methyl-6-(2-methyl-1-oxopropyl)-2-{[2-hydroxy-4,6-dimethoxy-3-methyl-5-(2-methyl-1-oxopropyl)phenyl]methyl}phenolate; 3,5-dihydroxy-6-methyl-4-(2-methyl-1-oxopropyl)-2-{[2-hydroxy-4,6-dimethoxy-3-methyl-5-(2-methyl-1-oxopropyl)phenyl]methyl}phenolate; and 3,5-dimethoxy-6-methyl-4-(2-methyl-1-oxopropyl)-2-{[2,4,6-trihydroxy-5-methyl-3-(2-methyl-1-oxopropyl)phenyl]methyl}phenolate.

59. The composition of any one of claims 1-19, wherein the molecule is beta-mangostin, which has CAS Registry Number 20931-37-7 and is related to The Merck Index monograph M7077; and the anion is selected from 6-hydroxy-2,8-diisoprenyl-3,7-dimethoxy-9-oxoxanthene-1-oxide and 8-hydroxy-1,7-diisoprenyl-2,6-dimethoxy-9-oxoxanthene-3-oxide.

60. The composition of any one of claims 1-19, wherein the molecule is beta-peltatin, which has CAS Registry Number 518-29-6 and is described in The Merck Index monograph M8455; and the anion is 8-oxo-9-(3,4,5-trimethoxyphenyl)-5a,6,8a,9-tetrahydro-5H-[2]benzofuro[5,6-f][1,3]benzodioxol-4-oxide.

61. The composition of any one of claims 1-19, wherein the molecule is beta-resorcylaldehyde, which has CAS Registry Number 95-01-2 and is described in The Merck Index monograph M9547; and the anion is selected from 4-formyl-3-hydroxyphenolate and 6-formyl-3-hydroxyphenolate.

62. The composition of any one of claims 1-19, wherein the molecule is beta-thujaplicin, which has CAS Registry Number 499-44-5 and is described in The Merck Index monograph M10814; and the anion is 3-isopropyl-7-oxocyclohepta-1,3,5-triene-1-oxide.

63. The composition of any one of claims 1-19, wherein the molecule is beta-tocopherol, which has CAS Registry Number 148-03-8 and is described in The Merck Index monograph M10924; and the anion is 2,5,8-trimethyl-2-(4,8,12-trimethyltridecyl)-3,4-dihydro-2H-chromene-6-oxide.

64. The composition of any one of claims 1-19, wherein the molecule is beta-tocotrienol, which has CAS Registry Number 490-23-3 and is described in The Merck Index monograph M10929; and the anion is 2,5,8-trimethyl-2-(4,8,12-trimethyltrideca-3,7,11-trieneyl)-3,4-dihydro-2H-chromene-6-oxide.

65. The composition of any one of claims 1-19, wherein the molecule is bifluranol, which has CAS Registry Number 34633-34-6 and is described in The Merck Index monograph M1025; and the anion is selected from 2-fluoro-4-[3-(3-fluoro-4-hydroxyphenyl)pent-2-yl]phenolate and 2-fluoro-4-[2-(3-fluoro-4-hydroxyphenyl)pent-3-yl]phenolate.

66. The composition of any one of claims 1-19, wherein the molecule is biochanin A, which has CAS Registry Number 491-80-5 and is described in The Merck Index monograph M2501; and the anion is selected from 5-hydroxy-4-oxo-3-(4-methoxyphenyl)-4H-chromene-7-oxide and 7-hydroxy-4-oxo-3-(4-methoxyphenyl)-4H-chromene-5-oxide.

67. The composition of any one of claims 1-19, wherein the molecule is bisphenol A, which has CAS Registry Number 80-05-7 and is described in The Merck Index monograph M2568; and the anion is 4-[2-(4-hydroxyphenyl)prop-2-yl]phenolate.

68. The composition of any one of claims 1-19, wherein the molecule is bisphenol B, which has CAS Registry Number 77-40-7 and is described in The Merck Index monograph M2569; and the anion is 4-[2-(4-hydroxyphenyl)but-2-yl]phenolate.

69. The composition of any one of claims 1-19, wherein the molecule is brazilin, which has CAS Registry Number 474-07-7 and is described in The Merck Index monograph M2642; and the anion is selected from 3,6a,10-trihydroxy-7,11b-dihydro-6H-indeno[2,1-c]chromene-9-oxide; 3,6a,9-trihydroxy-7,11b-dihydro-6H-indeno[2,1-c]chromene-10-oxide; and 6a,9,10-trihydroxy-7,11b-dihydro-6H-indeno[2,1-c]chromene-3-oxide.

70. The composition of any one of claims 1-19, wherein the molecule is brodifacoum, which has CAS Registry Number 56073-10-0 and is described in The Merck Index monograph M2656; and the anion is 2-oxo-3-{3-[4-(4-bromophenyl)phenyl]-1,2,3,4-tetrahydronaphthalen-1-yl}-2H-chromene-4-oxide.

71. The composition of any one of claims 1-19, wherein the molecule is bromadiolone, which has CAS Registry Number 28772-56-7 and is described in The Merck Index monograph M2659; and the anion is 2-oxo-3-{3-hydroxy-1-phenyl-3-[4-(4-bromophenyl)phenyl]propyl}-2H-chromene-4-oxide.

72. The composition of any one of claims 1-19, wherein the molecule is bromosalicylchloranilide, which has CAS Registry Number 3679-64-9 and is described in The Merck Index monograph M1039; and the anion is 4-bromo-2-[N-(4-chlorophenyl)carbamoyl]phenolate.

73. The composition of any one of claims 1-19, wherein the molecule is bromosaligenin, which has CAS Registry Number 2316-64-5 and is described in The Merck Index monograph M89; and the anion is 4-bromo-2-(hydroxymethyl)phenolate.

74. The composition of any one of claims 1-19, wherein the molecule is buparvaquone, which has CAS Registry Number 88426-33-9 and is described in The Merck Index monograph M2765; and the anion is 3-{[4-(tert-butyl)cyclohexyl]methyl}-1,4-dioxonaphthalene-2-oxide.

75. The composition of any one of claims 1-19, wherein the molecule is butopyronoxyl, which has CAS Registry Number 532-34-3 and is described in The Merck Index monograph M608; and the anion is 1-(butyloxy)-6-methyl-1,4-dioxohepta-2,5-diene-2-oxide.

76. The composition of any one of claims 1-19, wherein the molecule is butylated hydroxyanisole, which has CAS Registry Number 25013-16-5 and is described in The Merck Index monograph M2819; and the anion is selected from 2-(tert-butyl)-4-(methoxy)phenolate and 3-(tert-butyl)-4-(methoxy)phenolate.

77. The composition of any one of claims 1-19, wherein the molecule is butylated hydroxytoluene, which has CAS Registry Number 128-37-0 and is described in The Merck Index monograph M2820; and the anion is 2,6-bis(tert-butyl)-4-methylphenolate.

78. The composition of any one of claims 1-19, wherein the molecule is butylparaben, which has CAS Registry Number 94-26-8 and is described in The Merck Index monograph M2857; and the anion is 4-[(butoxy)carbonyl]phenolate.

79. The composition of any one of claims 1-19, wherein the molecule is cabenegrin A-I, which has CAS Registry Number 84297-59-6 and is described in The Merck Index monograph M2875; and the anion is 4-[4-hydroxy-3-methylbut-2-enyl]-6a,12a-dihydro-(1,3)dioxolo(5,6)[1]benzofuro[3,2-c]chromene-3(6H)-oxide.

80. The composition of any one of claims 1-19, wherein the molecule is cabenegrin A-II, which has CAS Registry Number 84297-60-9 and is described in The Merck Index monograph M2875; and the anion is 2-[4-hydroxy-3-methylbutyl]-6a,12a-dihydro-(1,3)dioxolo(5,6)[1]benzofuro[3,2-c]chromene-3(6H)-oxide.

81. The composition of any one of claims 1-19, wherein the molecule is cannabidiol, which has CAS Registry Number 13956-29-1 and is described in The Merck Index monograph M3020; and the anion is 3-hydroxy-5-pentyl-2-(3-methyl-6-isopropenylcyclohex-2-enyl)phenolate.

82. The composition of any one of claims 1-19, wherein the molecule is cannabidiphorol, which has CAS Registry Number 55824-13-0 and is related to The Merck Index monograph M3020; and the anion is 5-heptyl-3-hydroxy-2-(3-methyl-6-isopropenylcyclohex-2-enyl)phenolate.

83. The composition of any one of claims 1-19, wherein the molecule is cannabidivarin, which has CAS Registry Number 24274-48-4 and is related to The Merck Index monograph M3020; and the anion is 3-hydroxy-5-propyl-2-(3-methyl-6-isopropenylcyclohex-2-enyl)phenolate.

84. The composition of any one of claims 1-19, wherein the molecule is cannabinol, which has CAS Registry Number 521-35-7 and is described in The Merck Index monograph M3021; and the anion is 6,6,9-trimethyl-3-pentyl-benzo[c]chromene-1-oxide.

85. The composition of any one of claims 1-19, wherein the molecule is capsaicin, which has CAS Registry Number 404-86-4 and is described in The Merck Index monograph M3040; and the anion is 2-methoxy-4-{[N-(8-methyl-1-oxononan-6-enyl)amino]methyl}phenolate.

86. The composition of any one of claims 1-19, wherein the molecule is carvacrol, which has CAS Registry Number 499-75-2 and is described in The Merck Index monograph M3142; and the anion is 5-isopropyl-2-methylphenolate.

87. The composition of any one of claims 1-19, wherein the molecule is catechin, which has CAS Registry Number 154-23-4 and is described in The Merck Index monograph M3172; and the anion is selected from 3,5-dihydroxy-2-(3,4-dihydroxyphenyl)-3,4-dihydro-2H-chromene-7-oxide; 3,7-dihydroxy-2-(3,4-dihydroxyphenyl)-3,4-dihydro-2H-chromene-5-oxide; 2-hydroxy-4-(3,5,7-trihydroxy-3,4-dihydro-2H-chromen-2-yl)phenolate; and 6-hydroxy-3-(3,5,7-trihydroxy-3,4-dihydro-2H-chromen-2-yl)phenolate.

88. The composition of any one of claims 1-19, wherein the molecule is cavicularin, which has CAS Registry Number 178734-41-3 and is described in The Merck Index monograph M11850; and the anion is selected from 16,24-dihydroxy-14-oxahexacyclo[13.9.3.2^{10,13}.0^{2,7}.0^{19,27}.0^{22,26}]nonacosa-1(24),2,4,6,10,12,15,17,19(27),22,25,28-dodecaene-5-oxide; 5,24-dihydroxy-14-oxahexacyclo[13.9.3.2^{10,13}.0^{2,7}.0^{19,27}.0^{22,26}]nonacosa-1(24),2,4,6,10,12,15,17,19(27),22,25,28-dodecaene-16-oxide; and 5,16-dihydroxy-14-oxahexacyclo[13.9.3.2^{10,13}.0^{2,7}.0^{19,27}.0^{22,26}]nonacosa-1(24),2,4,6,10,12,15,17,19(27),22,25,28-dodecaene-24-oxide.

89. The composition of any one of claims 1-19, wherein the molecule is chavicol, which has CAS Registry Number 501-92-8 and is described in The Merck Index monograph M3319; and the anion is 4-(prop-2-enyl)phenolate.

90. The composition of any one of claims 1-19, wherein the molecule is chlorindanol, which has CAS Registry Number 145-94-8 and is described in The Merck Index monograph M110; and the anion is 7-chloro-2,3-dihydro-1H-indene-4-oxide.

91. The composition of any one of claims 1-19, wherein the molecule is chrysarobin, which has CAS Registry Number 491-58-7 and is described in The Merck Index monograph M3527; and the anion is selected from 8-hydroxy-3-methyl-9-oxo-10H-anthracen-1-oxide and 8-hydroxy-6-methyl-9-oxo-10H-anthracen-1-oxide.

92. The composition of any one of claims 1-19, wherein the molecule is chrysin, which has CAS Registry Number 480-40-0 and is described in The Merck Index monograph M3530; and the anion is selected from 5-hydroxy-4-oxo-2-phenyl-4H-chromene-7-oxide and 7-hydroxy-4-oxo-2-phenyl-4H-chromene-5-oxide.

93. The composition of any one of claims 1-19, wherein the molecule is clofoctol, which has CAS Registry Number 37693-01-9 and is described in The Merck Index monograph M3643; and the anion is 2-[(2,4-dichlorophenyl)methyl]-4-(2,4,4-trimethylpent-2-yl)phenolate.

94. The composition of any one of claims 1-19, wherein the molecule is clorophene, which has CAS Registry Number 120-32-1 and is described in The Merck Index monograph M3664; and the anion is 2-benzyl-4-chlorophenolate.

95. The composition of any one of claims 1-19, wherein the molecule is collinomycin, which has CAS Registry Number 1393-16-4 and is described in The Merck Index monograph M3736; and the anion is selected from 8-hydroxy-1-oxo-3-[(methoxy)carbonyl]-6-[2-(8-hydroxy-5,7-dimethoxy-4,9-dioxo-4,9-dihydro-benzo[f][1]benzofuran-2-yl)ethyl]-1H-isochromene-7-oxide; 7-hydroxy-1-oxo-3-[(methoxy)carbonyl]-6-[2-(8-hydroxy-5,7-dimethoxy-4,9-dioxo-4,9-dihydro-benzo[f][1]benzofuran-2-yl)ethyl]-1H-isochromene-8-oxide; and 5,7-dimethoxy-4,9-dioxo-2-({7,8-dihydroxy-1-oxo-3-[(methoxy)carbonyl]-1H-isochromen-6-yl}ethyl)-4,9-dihydro-benzo[f][1]benzofuran-8-oxide.

96. The composition of any one of claims 1-19, wherein the molecule is combretastatin, which has CAS Registry Number 82855-09-2 and is described in The Merck Index monograph M3747; and the anion is 2-methoxy-5-[2-hydroxy-2-(3,4,5-trimethoxyphenyl)ethyl]phenolate.

97. The composition of any one of claims 1-19, wherein the molecule is combretastatin A-1, which has CAS Registry Number 109971-63-3 and is described in The Merck Index monograph M3747; and the anion is selected from 2-hydroxy-3-methoxy-6-[2-(3,4,5-trimethoxyphenyl)ethenyl]phenolate and 6-hydroxy-2-methoxy-5-[2-(3,4,5-trimethoxyphenyl)ethenyl]phenolate.

98. The composition of any one of claims 1-19, wherein the molecule is combretastatin A-4, which has CAS Registry Number 117048-59-6 and is described in The Merck Index monograph M3747; and the anion is 2-methoxy-5-[2-(3,4,5-trimethoxyphenyl)ethenyl]phenolate.

99. The composition of any one of claims 1-19, wherein the molecule is combretastatin B-1, which has CAS Registry Number 109971-64-4 and is related to The Merck Index monograph M3747; and the anion is selected from 2-hydroxy-3-methoxy-6-[2-(3,4,5-trimethoxyphenyl)ethyl]phenolate and 6-hydroxy-2-methoxy-5-[2-(3,4,5-trimethoxyphenyl)ethyl]phenolate.

100. The composition of any one of claims 1-19, wherein the molecule is coniferyl alcohol, which has CAS Registry Number 458-35-5 and is described in The Merck Index monograph M3758; and the anion is 2-methoxy-4-(3-hydroxyprop-1-enyl)phenolate.

101. The composition of any one of claims 1-19, wherein the molecule is cotoin, which has CAS Registry Number 479-21-0 and is described in The Merck Index monograph M128; and the anion is 2-(phenylcarbonyl)-3-hydroxy-5-(methoxy)phenolate.

102. The composition of any one of claims 1-19, wherein the molecule is coumafuryl, which has CAS Registry Number 117-52-2 and is described in The Merck Index monograph M665; and the anion is 2-oxo-3-[1-(furan-2-yl)-3-oxobutyl]-2H-chromene-4-oxide.

103. The composition of any one of claims 1-19, wherein the molecule is coumestrol, which has CAS Registry Number 479-13-0 and is described in The Merck Index monograph M3822; and the anion is selected from 9-hydroxy-6-oxobenzofurano[3,2-c]chromen-3-oxide and 3-hydroxy-6-oxobenzofurano[3,2-c]chromen-9-oxide.

104. The composition of any one of claims 1-19, wherein the molecule is curcumin, which has CAS Registry Number 458-37-7 and is described in The Merck Index monograph M3933; and the anion is 2-methoxy-4-[3,5-dioxo-7-(4-hydroxy-3-methoxyphenyl)hepta-1,6-dieneyl]phenolate.

105. The composition of any one of claims 1-19, wherein the molecule is curvularin, which has CAS Registry Number 10140-70-2 and is described in The Merck Index monograph M3936; and the anion is selected from 13-hydroxy-5-methyl-3,11-dioxo-4-oxabicyclo[10.4.0]hexadeca-1(12),13,15-triene-15-oxide and 15-hydroxy-5-methyl-3,11-dioxo-4-oxabicyclo[10.4.0]hexadeca-1(12),13,15-triene-13-oxide.

106. The composition of any one of claims 1-19, wherein the molecule is cyclovalone, which has CAS Registry Number 579-23-7 and is described in The Merck Index monograph M1099; and the anion is 2-methoxy-4-[(2-oxo-3-{[4-hydroxy-3-(methyoxy)phenyl]methylidene}cyclohexylidene)methyl]phenolate.

107. The composition of any one of claims 1-19, wherein the molecule is daidzein, which has CAS Registry Number 486-66-8 and is described in The Merck Index monograph M4068; and the anion is selected from 4-oxo-3-(4-hydroxyphenyl)-4H-chromene-7-oxide and 4-(7-hydroxy-4-oxo-4H-chromen-3-yl)phenolate.

108. The composition of any one of claims 1-19, wherein the molecule is danthron, which has CAS Registry Number 117-10-2 and is described in The Merck Index monograph M4084; and the anion is 8-hydroxy-9,10-dioxoanthracene-1-oxide.

109. The composition of any one of claims 1-19, wherein the molecule is daphnetin, which has CAS Registry Number 486-35-1 and is described in The Merck Index monograph M4087; and the anion is selected from 8-hydroxy-2-oxo-2H-chromene-7-oxide and 7-hydroxy-2-oxo-2H-chromene-8-oxide.

110. The composition of any one of claims 1-19, wherein the molecule is datiscetin, which has CAS Registry Number 480-15-9 and is described in The Merck Index monograph M4102; and the anion is selected from 5,7-dihydroxy-4-oxo-2-(2-hydroxyphenyl)-4H-chromene-3-oxide; 3,7-dihydroxy-4-oxo-2-(2-hydroxyphenyl)-4H-chromene-5-oxide; 3,5-dihydroxy-4-oxo-2-(2-hydroxyphenyl)-4H-chromene-7-oxide; and 2-(3,5,7-trihydroxy-4-oxo-4H-chromen-2-yl)phenolate.

111. The composition of any one of claims 1-19, wherein the molecule is DBMC, which has CAS Registry Number 497-39-2 and is described in The Merck Index monograph M4110; and the anion is 5-methyl-2,4-bis(tert-butyl)phenolate.

112. The composition of any one of claims 1-19, wherein the molecule is deferiprone, which has CAS Registry Number 30652-11-0 and is described in The Merck Index monograph M4132; and the anion is 1,2-dimethyl-4-oxo-1H-pyridin-3-oxide.

113. The composition of any one of claims 1-19, wherein the molecule is dehydroequol, which has CAS Registry Number 81267-65-4 and is described in The Merck Index monograph M4146; and the anion is selected from 3-(4-hydroxyphenyl)-2H-chromene-7-oxide and 4-(7-hydroxy-2H-chromen-3-yl)phenolate.

114. The composition of any one of claims 1-19, wherein the molecule is delta8-tetrahydrocannabinol, which has CAS Registry Number 5957-75-5 and is related to The Merck Index monograph M10624; and the anion is 6,6,9-trimethyl-3-pentyl-6a,7,10,10a-tetrahydro-6H-benzo[c]chromene-1-oxide.

115. The composition of any one of claims 1-19, wherein the molecule is delta-tocopherol, which has CAS Registry Number 119-13-1 and is described in The Merck Index monograph M10926; and the anion is 2,8-dimethyl-2-(4,8,12-trimethyltridecyl)-3,4-dihydro-2H-chromene-6-oxide.

116. The composition of any one of claims 1-19, wherein the molecule is dexanabinol, which has CAS Registry Number 112924-45-5 and is described in The Merck Index monograph M4216; and the anion is 6,6-dimethyl-9-(hydroxymethyl)-3-(2-methyloct-2-yl)-6a,7,10,10a-tetrahydro-6H-benzo[c]chromene-1-oxide.

117. The composition of any one of claims 1-19, wherein the molecule is dichlorophen, which has CAS Registry Number 97-23-4 and is described in The Merck Index monograph M4351; and the anion is 4-chloro-2-[(3-chloro-6-hydroxyphenyl)methyl]phenolate.

118. The composition of any one of claims 1-19, wherein the molecule is dicoumarol, which has CAS Registry Number 66-76-2 and is described in The Merck Index monograph M4370; and the anion is 2-oxo-3-[(4-hydroxy-2-oxo-2H-chromen-3-yl)methyl]-2H-chromene-4-oxide.

119. The composition of any one of claims 1-19, wherein the molecule is dienestrol, which has CAS Registry Number 84-17-3 and is described in The Merck Index monograph M4385; and the anion is 4-[4-(4-hydroxyphenyl)hexa-2,4-dien-3-yl]phenolate.

120. The composition of any one of claims 1-19, wherein the molecule is diethylstilbestrol, which has CAS Registry Number 56-53-1 and is described in The Merck Index monograph M4418; and the anion is 4-[4-(4-hydroxyphenyl)hex-3-en-3-yl]phenolate.

121. The composition of any one of claims 1-19, wherein the molecule is diethylstilbestrol monobenzyl ether, which has CAS Registry Number 6202-26-2 and is related to The Merck Index monograph M4418; and the anion is 4-{4-[4-(benzyloxy)phenyl]hex-3-en-3-yl}phenolate.

122. The composition of any one of claims 1-19, wherein the molecule is difenacoum, which has CAS Registry Number 56073-07-5 and is described in The Merck Index monograph M4423; and the anion is 2-oxo-3-[3-(4-phenylphenyl)-1,2,3,4-tetrahydronaphthalen-1-yl]-2H-chromene-4-oxide.

123. The composition of any one of claims 1-19, wherein the molecule is dihydrocapsaicin, which has CAS Registry Number 19408-84-5 and is related to The Merck Index monograph M3040; and the anion is 2-methoxy-4-{[N-(8-methyl-1-oxononyl)amino]methyl}phenolate.

124. The composition of any one of claims 1-19, wherein the molecule is dihydrokaempferide, which has CAS Registry Number 3570-69-2 and is related to The Merck Index monograph M6592; and the anion is selected from 3,7-dihydroxy-4-oxo-2-(4-methoxyphenyl)-2,3-dihydro-4H-chromene-5-oxide and 3,5-dihydroxy-4-oxo-2-(4-methoxyphenyl)-2,3-dihydro-4H-chromene-7-oxide.

125. The composition of any one of claims 1-19, wherein the molecule is dihydrokaempferol, which has CAS Registry Number 724434-08-6 and is related to The Merck Index monograph M6592; and the anion is selected from 3,7-dihydroxy-4-oxo-2-(4-hydroxyphenyl)-2,3-dihydro-4H-chromene-5-oxide; 3,5-dihydroxy-4-oxo-2-(4-hydroxyphenyl)-2,3-dihydro-4H-chromene-7-oxide; and 4-(3,5,7-trihydroxy-4-oxo-2,3-dihydro-4H-chromen-2-yl)phenolate.

126. The composition of any one of claims 1-19, wherein the molecule is dihydroresveratrol, which has CAS Registry Number 58436-28-5 and is related to The Merck Index monograph M9549; and the anion is selected from 4-[2-(3,5-dihydroxyphenyl)ethyl]phenolate and 3-hydroxy-5-[2-(4-hydroxyphenyl)ethyl]phenolate.

127. The composition of any one of claims 1-19, wherein the molecule is dimethylheptyl cannabidiol, which has CAS Registry Number 97452-63-6 and is related to The Merck Index monograph M3020; and the anion is 3-hydroxy-2-(3-methyl-6-isopropenylcyclohex-2-enyl)-5-(2-methyloctan-2-yl)phenolate.

128. The composition of any one of claims 1-19, wherein the molecule is dimethylstilbestrol, which has CAS Registry Number 552-80-7 and is related to The Merck Index monograph M4418; and the anion is 4-[3-(4-hydroxyphenyl)but-2-en-2-yl]phenolate.

129. The composition of any one of claims 1-19, wherein the molecule is dioxybenzone, which has CAS Registry Number 131-53-3 and is described in The Merck Index monograph M4602; and the anion is selected from 2-[(2-hydroxy-4-methoxyphenyl)carbonyl]phenolate and 3-methoxy-6-[(2-hydroxyphenyl)carbonyl]phenolate.

130. The composition of any one of claims 1-19, wherein the molecule is dobutamine, which has CAS Registry Number 34368-04-2 and is described in The Merck Index monograph M4710; and the anion is selected from 2-hydroxy-4-(2-{N-[4-(4-hydroxyphenyl)but-2-yl]amino}ethyl)phenolate; 2-hydroxy-5-(2-{N-[4-(4-hydroxyphenyl)but-2-yl]amino}ethyl)phenolate; and 4-(3-{N-[2-(3,4-dihydroxyphenyl)ethyl]amino}butyl)phenolate.

131. The composition of any one of claims 1-19, wherein the molecule is embelin, which has CAS Registry Number 550-24-3 and is described in The Merck Index monograph M4883; and the anion is selected from 5-hydroxy-3-undecyl-1,4-benzoquinone-2-oxide and 5-hydroxy-6-undecyl-1,4-benzoquinone-3-oxide.

132. The composition of any one of claims 1-19, wherein the molecule is emodin, which has CAS Registry Number 518-82-1 and is described in The Merck Index monograph M4888; and the anion is selected from 4,5-dihydroxy-7-methyl-9,10-dioxoanthracene-2-oxide; 3,8-dihydroxy-6-methyl-9,10-dioxoanthracene-1-oxide; and 6,8-dihydroxy-3-methyl-9,10-dioxoanthracene-1-oxide.

133. The composition of any one of claims 1-19, wherein the molecule is enterolactone, which has CAS Registry Number 78473-71-9 and is described in The Merck Index monograph M4922; and the anion is selected from 3-({4-oxo-5-[(3-hydroxyphenyl)methyl]-3-oxacyclopentyl}methyl)phenolate and 3-({2-oxo-5-[(3-hydroxyphenyl)methyl]-3-oxacyclopentyl}methyl)phenolate.

134. The composition of any one of claims 1-19, wherein the molecule is epicocconone, which has CAS Registry Number 371163-96-1 and is described in The Merck Index monograph M4940; and the anion is 3-oxo-1-[9a-methyl-2,9-dioxo-6-(hydroxymethyl)-5,6-dihydro-2H-furo[3,2-g]isochromen-3-yl]deca-1,4,6,8-tetraene-1-oxide.

135. The composition of any one of claims 1-19, wherein the molecule is epigallocatechin gallate, which has CAS Registry Number 989-51-5 and is described in The Merck Index monograph M4843; and the anion is selected from 2,3-dihydroxy-5-({[5,7-dihydroxy-2-(3,4,5-trihydroxyphenyl)-3,4-dihydro-2H-chromen-3-yl]oxy}carbonyl)phenolate; 2,6-dihydroxy-4-({[5,7-dihydroxy-2-(3,4,5-trihydroxyphenyl)-3,4-dihydro-2H-chromen-3-yl]oxy}carbonyl)phenolate; 7-hydroxy-2-(3,4,5-trihydroxyphenyl)-3-{[(3,4,5-trihydroxyphenyl)carbonyl]oxy}-3,4-dihydro-2H-chromene-5-oxide; 5-hydroxy-2-(3,4,5-trihydroxyphenyl)-3-{[(3,4,5-trihydroxyphenyl)carbonyl]oxy}-3,4-dihydro-2H-chromene-7-oxide; 2,3-dihydroxy-5-(5,7-dihydroxy-3-{[(3,4,5-trihydroxyphenyl)carbonyl]oxy}-3,4-dihydro-2H-chromen-2-yl)phenolate; and 2,6-dihydroxy-4-(5,7-dihydroxy-3-{[(3,4,5-trihydroxyphenyl)carbonyl]oxy}-3,4-dihydro-2H-chromen-2-yl)phenolate.

136. The composition of any one of claims 1-19, wherein the molecule is equilenin, which has CAS Registry Number 517-09-9 and is described in The Merck Index monograph M4969; and the anion is 13-methyl-17-oxo-12,14,15,16-tetrahydro-11H-cyclopenta[a]phenanthrene-3-oxide.

137. The composition of any one of claims 1-19, wherein the molecule is equilin, which has CAS Registry Number 474-86-2 and is described in The Merck Index monograph M4970; and the anion is 13-methyl-17-oxo-9,11,12,14,15,16-hexahydro-6H-cyclopenta[a]phenanthrene-3-oxide.

138. The composition of any one of claims 1-19, wherein the molecule is equol, which has CAS Registry Number 531-95-3 and is described in The Merck Index monograph M4971; and the anion is selected from 3-(4-hydroxyphenyl)-3,4-dihydro-2H-chromene-7-oxide and 4-(7-hydroxy-3,4-dihydro-2H-chromen-3-yl)phenolate.

139. The composition of any one of claims 1-19, wherein the molecule is ergoflavin, which has CAS Registry Number 3101-51-7 and is described in The Merck Index monograph M4979; and the anion is 4,9a-dihydroxy-3-methyl-9-oxo-1,4a-(oxycarbonyl-O,1-diyl)-7-[4,8,9a-trihydroxy-3-methyl-9-oxo-1,4a-(oxycarbonyl-O,1-diyl)-1,2,3,4-tetrahydroxanthen-7-yl]-1,2,3,4-tetrahydroxanthene-8-oxide.

140. The composition of any one of claims 1-19, wherein the molecule is eriodictyol, which has CAS Registry Number 552-58-9 and is described in The Merck Index monograph M4996; and the anion is selected from 5-hydroxy-4-oxo-2-(3,4-dihydroxyphenyl)-2,3-dihydro-4H-chromene-7-oxide; 7-hydroxy-4-oxo-2-(3,4-dihydroxyphenyl)-2,3-dihydro-4H-chromene-5-oxide; 2-hydroxy-4-(5,7-dihydroxy-4-oxo-2,3-dihydro-4H-chromen-2-yl)phenolate; and 2-hydroxy-5-(5,7-dihydroxy-4-oxo-2,3-dihydro-4H-chromen-2-yl)phenolate.

141. The composition of any one of claims 1-19, wherein the molecule is esculetin, which has CAS Registry Number 305-01-1 and is described in The Merck Index monograph M5022; and the anion is selected from 6-hydroxy-2-oxo-2H-chromene-7-oxide and 7-hydroxy-2-oxo-2H-chromene-6-oxide.

142. The composition of any one of claims 1-19, wherein the molecule is estradiol, which has CAS Registry Number 50-28-2 and is described in The Merck Index monograph M5028; and the anion is 17-hydroxy-13-methyl-6,7,8,9,11,12,14,15,16,17-decahydrocyclopenta[a]phenanthrene-3-oxide.

143. The composition of any one of claims 1-19, wherein the molecule is estradiol 17p-cyclopentanepropanoate, which has CAS Registry Number 313-06-4 and is described in The Merck Index monograph M5028; and the anion is 13-methyl-17-{[1-oxo-3-(cyclopentyl)propyl]oxy}-6,7,8,9,11,12,14,15,16,17-decahydrocyclopenta[a]phenanthrene-3-oxide.

144. The composition of any one of claims 1-19, wherein the molecule is estradiol enanthate, which has CAS Registry Number 4956-37-0 and is described in The Merck Index monograph M5028; and the anion is 13-methyl-17-[(1-oxoheptyl)oxy]-6,7,8,9,11,12,14,15,16,17-decahydrocyclopenta[a]phenanthrene-3-oxide.

145. The composition of any one of claims 1-19, wherein the molecule is estradiol undecylate, which has CAS Registry Number 3571-53-7 and is described in The Merck Index monograph M5028; and the anion is 13-methyl-17-[(1-oxoundecyl)oxy]-6,7,8,9,11,12,14,15,16,17-decahydrocyclopenta[a]phenanthrene-3-oxide.

146. The composition of any one of claims 1-19, wherein the molecule is estradiol valerate, which has CAS Registry Number 979-32-8 and is described in The Merck Index monograph M5028; and the anion is 13-methyl-17-[(1-oxopentyl)oxy]-6,7,8,9,11,12,14,15,16,17-decahydrocyclopenta[a]phenanthrene-3-oxide.

147. The composition of any one of claims 1-19, wherein the molecule is estriol, which has CAS Registry Number 50-27-1 and is described in The Merck Index monograph M5032; and the anion is 16,17-dihydroxy-13-methyl-6,7,8,9,11,12,14,15,16,17-decahydrocyclopenta[a]phenanthrene-3-oxide.

148. The composition of any one of claims 1-19, wherein the molecule is estrone, which has CAS Registry Number 53-16-7 and is described in The Merck Index monograph M5033; and the anion is 13-methyl-17-oxo-7,8,9,11,12,14,15,16-octahydro-6H-cyclopenta[a]phenanthrene-3-oxide.

149. The composition of any one of claims 1-19, wherein the molecule is eta-tocopherol, which has CAS Registry Number 91-86-1 and is described in The Merck Index monograph M501; and the anion is 2,7-dimethyl-2-(4,8,12-trimethyltridecyl)-3,4-dihydro-2H-chromene-6-oxide.

150. The composition of any one of claims 1-19, wherein the molecule is ethinyl estradiol, which has CAS Registry Number 57-63-6 and is described in The Merck Index monograph M5058; and the anion is 17-ethynyl-17-hydroxy-13-methyl-7,8,9,11,12,14,15,16-octahydro-6H-cyclopenta[a]phenanthrene-3-oxide.

151. The composition of any one of claims 1-19, wherein the molecule is ethyl biscoumacetate, which has CAS Registry Number 548-00-5 and is described in The Merck Index monograph M5095; and the anion is 2-oxo-3-[2-(ethoxy)-2-oxo-1-(4-hydroxy-2-oxo-2H-chromen-3-yl)ethyl]-2H-chromene-4-oxide.

152. The composition of any one of claims 1-19, wherein the molecule is ethyl maltol, which has CAS Registry Number 4940-11-8 and is described in The Merck Index monograph M5149; and the anion is 2-ethyl-4-oxo-4H-pyran-3-oxide.

153. The composition of any one of claims 1-19, wherein the molecule is ethyl vanillin, which has CAS Registry Number 121-32-4 and is described in The Merck Index monograph M5178; and the anion is 2-ethoxy-4-formylphenolate.

154. The composition of any one of claims 1-19, wherein the molecule is ethylidene dicoumarol, which has CAS Registry Number 1821-16-5 and is described in The Merck Index monograph M5137; and the anion is 2-oxo-3-[1-(4-hydroxy-2-oxo-2H-chromen-3-yl)ethyl]-2H-chromene-4-oxide.

155. The composition of any one of claims 1-19, wherein the molecule is ethylparaben, which has CAS Registry Number 120-47-8 and is described in The Merck Index monograph M5160; and the anion is 4-[(ethoxy)carbonyl]phenolate.

156. The composition of any one of claims 1-19, wherein the molecule is eugenol, which has CAS Registry Number 97-53-0 and is described in The Merck Index monograph M5210; and the anion is 2-methoxy-4-(prop-2-enyl)phenolate.

157. The composition of any one of claims 1-19, wherein the molecule is euparin, which has CAS Registry Number 532-48-9 and is described in The Merck Index monograph M5212; and the anion is 5-acetyl-2-isopropenyl-1-benzofuran-6-oxide.

158. The composition of any one of claims 1-19, wherein the molecule is eupatorin, which has CAS Registry Number 855-96-9 and is described in The Merck Index monograph M5213; and the anion is selected from 6,7-dimethoxy-4-oxo-2-(3-hydroxy-4-methoxyphenyl)-4H-chromene-5-oxide and 2-methoxy-5-(5-hydroxy-6,7-dimethoxy-4-oxo-4H-chromen-2-yl)phenolate.

159. The composition of any one of claims 1-19, wherein the molecule is fenoterol, which has CAS Registry Number 13392-18-2 and is described in The Merck Index monograph M5282; and the anion is selected from 3-hydroxy-5-(1-hydroxy-2-{N-[1-(4-hydroxyphenyl)prop-2-yl]amino}ethyl)phenolate and 4-(2-{N-[2-hydroxy-2-(3,5-dihydroxyphenyl)ethyl]amino}propyl)phenolate.

160. The composition of any one of claims 1-19, wherein the molecule is fenretinide, which has CAS Registry Number 65646-68-6 and is described in The Merck Index monograph M5295; and the anion is 4-{N-[3,7-dimethyl-1-oxo-9-(2,6,6-trimethylcyclohex-1-enyl)nona-2,4,6,8-tetraenyl]amino}phenolate.

161. The composition of any one of claims 1-19, wherein the molecule is fisetin, which has CAS Registry Number 528-48-3 and is described in The Merck Index monograph M5389; and the anion is selected from 7-hydroxy-4-oxo-2-(3,4-dihydroxyphenyl)-4H-chromene-3-oxide; 3-hydroxy-4-oxo-2-(3,4-dihydroxyphenyl)-4H-chromene-7-oxide; 2-hydroxy-4-(3,7-dihydroxy-4-oxo-4H-chromen-2-yl)phenolate; and 2-hydroxy-5-(3,7-dihydroxy-4-oxo-4H-chromen-2-yl)phenolate.

162. The composition of any one of claims 1-19, wherein the molecule is flavopurpurin, which has CAS Registry Number 82-29-1 and is related to The Merck Index monograph M9326; and the anion is selected from 2,6-dihydroxy-9,10-dioxoanthracene-1-oxide; 1,6-dihydroxy-9,10-dioxoanthracene-2-oxide; and 5,6-dihydroxy-9,10-dioxoanthracene-2-oxide.

163. The composition of any one of claims 1-19, wherein the molecule is flocoumafen, which has CAS Registry Number 90035-08-8 and is described in The Merck Index monograph M5404; and the anion is 2-oxo-3-{3-[4-({[4-(trifluoromethyl)phenyl]methyl}oxy)phenyl]-1,2,3,4-tetrahydronaphthalen-1-yl}-2H-chromene-4-oxide.

164. The composition of any one of claims 1-19, wherein the molecule is flopropione, which has CAS Registry Number 2295-58-1 and is described in The Merck Index monograph M5408; and the anion is selected from 3,5-dihydroxy-2-(1-oxopropyl)phenolate and 3,5-dihydroxy-4-(1-oxopropyl)phenolate.

165. The composition of any one of claims 1-19, wherein the molecule is fluorescein, which has CAS Registry Number 2321-07-5 and is described in The Merck Index monograph M5462; and the anion is 6′-hydroxy-3-oxospiro(2-benzofuran-1,9′-xanthene)-3′-oxide.

166. The composition of any one of claims 1-19, wherein the molecule is formestane, which has CAS Registry Number 566-48-3 and is described in The Merck Index monograph M5538; and the anion is 10,13-methyl-3,17-dioxo-2,6,7,8,9,11,12,14,15,16-decahydro-1H-cyclopenta[a]phenanthrene-4-oxide.

167. The composition of any one of claims 1-19, wherein the molecule is formononetin, which has CAS Registry Number 485-72-3 and is described in The Merck Index monograph M5541; and the anion is 4-oxo-3-(4-methoxyphenyl)-4H-chromene-7-oxide.

168. The composition of any one of claims 1-19, wherein the molecule is fraxetin, which has CAS Registry Number 574-84-5 and is described in The Merck Index monograph M5564; and the anion is selected from 8-hydroxy-6-methoxy-2-oxo-2H-chromene-7-oxide and 7-hydroxy-6-methoxy-2-oxo-2H-chromene-8-oxide.

169. The composition of any one of claims 1-19, wherein the molecule is fulvestrant, which has CAS Registry Number 129453-61-8 and is described in The Merck Index monograph M5583; and the anion is 17-hydroxy-13-methyl-7-{9-[(4,4,5,5,5-pentafluoropentyl)sulfinyl]nonyl}-6,7,8,9,11,12,14,15,16,17-decahydrocyclopenta[a]phenanthrene-3-oxide.

170. The composition of any one of claims 1-19, wherein the molecule is fumigatin, which has CAS Registry Number 484-89-9 and is described in The Merck Index monograph M5587; and the anion is 6-methyl-3-methoxy-1,4-benzoquinone-2-oxide.

171. The composition of any one of claims 1-19, wherein the molecule is fusarubin, which has CAS Registry Number 1702-77-8 and is described in The Merck Index monograph M5614; and the anion is selected from 3,10-dihydroxy-3-methyl-7-methoxy-6,9-dioxo-1,4-dihydrobenzo[g]isochromene-5-oxide and 3,5-dihydroxy-3-methyl-7-methoxy-6,9-dioxo-1,4-dihydrobenzo[g]isochromene-10-oxide.

172. The composition of any one of claims 1-19, wherein the molecule is fuscin, which has CAS Registry Number 83-85-2 and is described in The Merck Index monograph M741; and the anion is 4,8,8-trimethyl-2,6-dioxo-9,10-dihydro-4H-pyrano[3,2-f]isochromene-5-oxide.

173. The composition of any one of claims 1-19, wherein the molecule is fustin, which has CAS Registry Number 20725-03-5 and is described in The Merck Index monograph M5617; and the anion is selected from 3-hydroxy-4-oxo-2-(3,4-dihydroxyphenyl)-2,3-dihydro-4H-chromene-7-oxide; 2-hydroxy-4-(3,7-dihydroxy-4-oxo-2,3-dihydro-4H-chromen-2-yl)phenolate; and 2-hydroxy-5-(3,7-dihydroxy-4-oxo-2,3-dihydro-4H-chromen-2-yl)phenolate.

174. The composition of any one of claims 1-19, wherein the molecule is galangin, which has CAS Registry Number 548-83-4 and is described in The Merck Index monograph M5639; and the anion is selected from 3,5-dihydroxy-4-oxo-2-phenyl-4H-chromene-7-oxide; 3,7-dihydroxy-4-oxo-2-phenyl-4H-chromene-5-oxide; and 5,7-dihydroxy-4-oxo-2-phenyl-4H-chromene-3-oxide.

175. The composition of any one of claims 1-19, wherein the molecule is gallein, which has CAS Registry Number 2103-64-2 and is described in The Merck Index monograph M5644; and the anion is selected from 4′,5′,6′-trihydroxy-1-oxospiro[2-benzofuran-3,9′-xanthene]-3′-oxide and 3′,5′,6′-trihydroxy-1-oxospiro[2-benzofuran-3,9′-xanthene]-4′-oxide.

176. The composition of any one of claims 1-19, wherein the molecule is gallocatechin, which has CAS Registry Number 970-73-0 and is related to The Merck Index monograph M3172; and the anion is selected from 3,5-dihydroxy-2-(3,4,5-trihydroxyphenyl)-3,4-dihydro-2H-chromene-7-oxide; 3,7-dihydroxy-2-(3,4,5-trihydroxyphenyl)-3,4-dihydro-2H-chromene-5-oxide; 2,6-dihydroxy-4-(3,5,7-trihydroxy-3,4-dihydro-2H-chromen-2-yl)phenolate; and 2,3-dihydroxy-5-(3,5,7-trihydroxy-3,4-dihydro-2H-chromen-2-yl)phenolate.

177. The composition of any one of claims 1-19, wherein the molecule is gamma-mangostin, which has CAS Registry Number 31271-07-5 and is related to The Merck Index monograph M7077; and the anion is selected from 3,6,7-trihydroxy-2,8-diisoprenyl-9-oxoxanthene-1-oxide; 3,6,8-trihydroxy-1,7-diisoprenyl-9-oxoxanthene-2-oxide; 1,6,7-trihydroxy-2,8-diisoprenyl-9-oxoxanthene-3-oxide; and 2,6,8-trihydroxy-1,7-diisoprenyl-9-oxoxanthene-3-oxide.

178. The composition of any one of claims 1-19, wherein the molecule is gamma-thujaplicin, which has CAS Registry Number 672-76-4 and is related to The Merck Index monograph M10814; and the anion is 4-isopropyl-7-oxocyclohepta-1,3,5-triene-1-oxide.

179. The composition of any one of claims 1-19, wherein the molecule is gamma-tocopherol, which has CAS Registry Number 54-28-4 and is described in The Merck Index monograph M10925; and the anion is 2,7,8-trimethyl-2-(4,8,12-trimethyltridecyl)-3,4-dihydro-2H-chromene-6-oxide.

180. The composition of any one of claims 1-19, wherein the molecule is gardenin A, which has CAS Registry Number 21187-73-5 and is described in The Merck Index monograph M5669; and the anion is 6,7,8-trimethoxy-4-oxo-2-(3,4,5-trimethoxyphenyl)-4H-chromene-5-oxide.

181. The composition of any one of claims 1-19, wherein the molecule is gardenin B, which has CAS Registry Number 2798-20-1 and is described in The Merck Index monograph M5669; and the anion is 6,7,8-trimethoxy-4-oxo-2-(4-methoxyphenyl)-4H-chromene-5-oxide.

182. The composition of any one of claims 1-19, wherein the molecule is gardenin C, which has CAS Registry Number 29550-05-8 and is described in The Merck Index monograph M5669; and the anion is selected from 6,7,8-trimethoxy-4-oxo-2-(3-hydroxy-4,5-dimethoxyphenyl)-4H-chromene-5-oxide and 2,3-dimethoxy-5-(5-hydroxy-6,7,8-trimethoxy-4-oxo-4H-chromen-2-yl)phenolate.

183. The composition of any one of claims 1-19, wherein the molecule is gardenin D, which has CAS Registry Number 29202-00-4 and is described in The Merck Index monograph M5669; and the anion is selected from 6,7,8-trimethoxy-4-oxo-2-(3-hydroxy-4-methoxyphenyl)-4H-chromene-5-oxide and 2-methoxy-5-(5-hydroxy-6,7,8-trimethoxy-4-oxo-4H-chromen-2-yl)phenolate.

184. The composition of any one of claims 1-19, wherein the molecule is gardenin E, which has CAS Registry Number 29550-07-0 and is described in The Merck Index monograph M5669; and the anion is selected from 6,7,8-trimethoxy-4-oxo-2-(3,5-dihydroxy-4-methoxyphenyl)-4H-chromene-5-oxide and 3-hydroxy-2-methoxy-5-(5-hydroxy-6,7,8-trimethoxy-4-oxo-4H-chromen-2-yl)phenolate.

185. The composition of any one of claims 1-19, wherein the molecule is genistein, which has CAS Registry Number 446-72-0 and is described in The Merck Index monograph M5696; and the anion is selected from 5-hydroxy-4-oxo-3-(4-hydroxyphenyl)-4H-chromene-7-oxide; 7-hydroxy-4-oxo-3-(4-hydroxyphenyl)-4H-chromene-5-oxide; and 4-(5,7-dihydroxy-4-oxo-4H-chromen-3-yl)phenolate.

186. The composition of any one of claims 1-19, wherein the molecule is gentisin, which has CAS Registry Number 437-50-3 and is described in The Merck Index monograph M5704; and the anion is selected from 8-hydroxy-6-methoxy-9-oxoxanthene-2-oxide and 7-hydroxy-3-methoxy-9-oxoxanthene-1-oxide.

187. The composition of any one of claims 1-19, wherein the molecule is gentisyl alcohol, which has CAS Registry Number 495-08-9 and is described in The Merck Index monograph M743; and the anion is selected from 4-hydroxy-2-(hydroxymethyl)phenolate and 4-hydroxy-3-(hydroxymethyl)phenolate.

188. The composition of any one of claims 1-19, wherein the molecule is geranylhydroquinone, which has CAS Registry Number 10457-66-6 and is described in The Merck Index monograph M5709; and the anion is selected from 2-geranyl-4-hydroxyphenolate and 3-geranyl-4-hydroxyphenolate.

189. The composition of any one of claims 1-19, wherein the molecule is gossypol, which has CAS Registry Number 303-45-7 and is described in The Merck Index monograph M5834; and the anion is selected from 8-formyl-6,7-dihydroxy-5-isopropyl-3-methyl-2-(8-formyl-1,6,7-trihydroxy-5-isopropyl-3-methylnapthalen-2-yl)napthalene-1-oxide; 1-formyl-3,8-dihydroxy-4-isopropyl-6-methyl-7-(8-formyl-1,6,7-trihydroxy-5-isopropyl-3-methylnapthalen-2-yl)napthalene-2-oxide; and 4-formyl-3,5-dihydroxy-1-isopropyl-7-methyl-6-(8-formyl-1,6,7-trihydroxy-5-isopropyl-3-methylnapthalen-2-yl)napthalene-2-oxide.

190. The composition of any one of claims 1-19, wherein the molecule is guaiacol, which has CAS Registry Number 90-05-1 and is described in The Merck Index monograph M5859; and the anion is 2-methoxyphenolate.

191. The composition of any one of claims 1-19, wherein the molecule is hematein, which has CAS Registry Number 475-25-2 and is described in The Merck Index monograph M5939; and the anion is selected from 3,4,6a-trihydroxy-9-oxo-6,7-dihydroindeno[2,1-c]chromene-10-oxide; 3,6a,10-trihydroxy-9-oxo-6,7-dihydroindeno[2,1-c]chromene-4-oxide; and 4,6a,10-trihydroxy-9-oxo-6,7-dihydroindeno[2,1-c]chromene-3-oxide.

192. The composition of any one of claims 1-19, wherein the molecule is hematoxylin, which has CAS Registry Number 517-28-2 and is described in The Merck Index monograph M5942; and the anion is selected from 3,4,6a,9-tetrahydroxy-7,11b-dihydro-6H-indeno[2,1-c]chromene-10-oxide; 3,4,6a,10-tetrahydroxy-7,11b-dihydro-6H-indeno[2,1-c]chromene-9-oxide; 3,6a,9,10-tetrahydroxy-7,11b-dihydro-6H-indeno[2,1-c]chromene-4-oxide; and 4,6a,9,10-tetrahydroxy-7,11b-dihydro-6H-indeno[2,1-c]chromene-3-oxide.

193. The composition of any one of claims 1-19, wherein the molecule is heminordihydroguaiaretic acid, which has CAS Registry Number 54473-24-4 and is related to The Merck Index monograph M8052; and the anion is selected from 2-hydroxy-4-[2,3-dimethyl-4-(4-hydroxy-3-methoxyphenyl)butyl]phenolate; 2-hydroxy-5-[2,3-dimethyl-4-(4-hydroxy-3-methoxyphenyl)butyl]phenolate; and 2-methoxy-4-[2,3-dimethyl-4-(3,4-dihydroxyphenyl)butyl]phenolate.

194. The composition of any one of claims 1-19, wherein the molecule is herqueinone, which has CAS Registry Number 26871-30-7 and is described in The Merck Index monograph M5973; and the anion is selected from 6,7a-dihydroxy-5-methoxy-1,8,8,9-tetramethyl-3,7-dioxo-9H-phenaleno[1,2-b]furan-4-oxide and 4,7a-dihydroxy-5-methoxy-1,8,8,9-tetramethyl-3,7-dioxo-9H-phenaleno[1,2-b]furan-6-oxide.

195. The composition of any one of claims 1-19, wherein the molecule is hesperetin, which has CAS Registry Number 520-33-2 and is described in The Merck Index monograph M5975; and the anion is selected from 5-hydroxy-4-oxo-2-(3-hydroxy-4-methoxyphenyl)-2,3-dihydro-4H-chromene-7-oxide; 7-hydroxy-4-oxo-2-(3-hydroxy-4-methoxyphenyl)-2,3-dihydro-4H-chromene-5-oxide; and 2-methoxy-5-(5,7-dihydroxy-4-oxo-2,3-dihydro-4H-chromen-2-yl]phenolate.

196. The composition of any one of claims 1-19, wherein the molecule is hexestrol, which has CAS Registry Number 84-16-2 and is described in The Merck Index monograph M6007; and the anion is 4-[4-(4-hydroxyphenyl)hex-3-yl]phenolate.

197. The composition of any one of claims 1-19, wherein the molecule is homocapsaicin I, which has CAS Registry Number 58493-48-4 and is related to The Merck Index monograph M3040; and the anion is 2-methoxy-4-{[N-(9-methyl-1-oxodec-6-enyl)amino]methyl}phenolate.

198. The composition of any one of claims 1-19, wherein the molecule is homocapsaicin II, which has CAS Registry Number 71240-51-2 and is related to The Merck Index monograph M3040; and the anion is 2-methoxy-4-{[N-(8-methyl-1-oxodec-6-enyl)amino]methyl}phenolate.

199. The composition of any one of claims 1-19, wherein the molecule is homodihydrocapsaicin, which has CAS Registry Number 20279-06-5 and is related to The Merck Index monograph M3040; and the anion is 2-methoxy-4-{[N-(9-methyl-1-oxodecyl)amino]methyl}phenolate.

200. The composition of any one of claims 1-19, wherein the molecule is homoeriodictyol, which has CAS Registry Number 446-71-9 and is described in The Merck Index monograph M6044; and the anion is selected from 5-hydroxy-4-oxo-2-(4-hydroxy-3-methoxyphenyl)-2,3-dihydro-4H-chromene-7-oxide; 7-hydroxy-4-oxo-2-(4-hydroxy-3-methoxyphenyl)-2,3-dihydro-4H-chromene-5-oxide; and 2-methoxy-4-(5,7-dihydroxy-4-oxo-2,3-dihydro-4H-chromen-2-yl)phenolate.

201. The composition of any one of claims 1-19, wherein the molecule is homosalate, which has CAS Registry Number 118-56-9 and is described in The Merck Index monograph M6047; and the anion is 2-{[(3,3,5-trimethylcyclohexyl)oxy]carbonyl}phenolate.

202. The composition of any one of claims 1-19, wherein the molecule is homovanillyl alcohol, which has CAS Registry Number 2380-78-1 and is related to The Merck Index monograph M11390; and the anion is 4-(2-hydroxyethyl)-2-methoxyphenolate.

203. The composition of any one of claims 1-19, wherein the molecule is honokiol, which has CAS Registry Number 35354-74-6 and is described in The Merck Index monograph M6051; and the anion is selected from 2-(prop-2-enyl)-4-[2-hydroxy-5-(prop-2-enyl)phenyl]phenolate and 4-(prop-2-enyl)-2-[4-hydroxy-3-(prop-2-enyl)phenyl]phenolate.

204. The composition of any one of claims 1-19, wherein the molecule is hydrocarbostyril, which has CAS Registry Number 553-03-7 and is described in The Merck Index monograph M6087; and the anion is 3,4-dihydroquinoline-2-oxide.

205. The composition of any one of claims 1-19, wherein the molecule is hydroxytyrosol, which has CAS Registry Number 10597-60-1 and is described in The Merck Index monograph M6157; and the anion is selected from 2-hydroxy-4-(2-hydroxyethyl)phenolate and 2-hydroxy-5-(2-hydroxyethyl)phenolate.

206. The composition of any one of claims 1-19, wherein the molecule is hymecromone, which has CAS Registry Number 90-33-5 and is described in The Merck Index monograph M6164; and the anion is selected from 4-methyl-2-oxo-2H-chromene-7-oxide and 7-oxo-4-methyl-7H-chromene-2-oxide.

207. The composition of any one of claims 1-19, wherein the molecule is ilimaquinone, which has CAS Registry Number 71678-03-0 and is described in The Merck Index monograph M6209; and the anion is 5-methoxy-3-[(1,2,4a-trimethyl-5-methylidene-3,4,6,7,8,8a-hexahydro-2H-naphthalen-1-yl)methyl]-1,4-benzoquinone-2-oxide.

208. The composition of any one of claims 1-19, wherein the molecule is irigenin, which has CAS Registry Number 548-76-5 and is described in The Merck Index monograph M6404; and the anion is selected from 5-hydroxy-6-methoxy-4-oxo-3-(3-hydroxy-4,5-dimethoxyphenyl)-4H-chromene-7-oxide; 7-hydroxy-6-methoxy-4-oxo-3-(3-hydroxy-4,5-dimethoxyphenyl)-4H-chromene-5-oxide; and 2,3-dimethoxy-5-(5,7-dihydroxy-6-methoxy-4-oxo-4H-chromen-3-yl)phenolate.

209. The composition of any one of claims 1-19, wherein the molecule is irisolone, which has CAS Registry Number 3301-68-6 and is described in The Merck Index monograph M248; and the anion is 4-(9-methoxy-8-oxo-[1,3]diolxolo[4,5-g]chromen-7-yl)phenolate.

210. The composition of any one of claims 1-19, wherein the molecule is isoestradiol, which has CAS Registry Number 517-04-4 and is described in The Merck Index monograph M6482; and the anion is 17-hydroxy-13-methyl-6,7,8,9,11,12,14,15,16,17-decahydrocyclopenta[a]phenanthrene-3-oxide.

211. The composition of any one of claims 1-19, wherein the molecule is isoeugenol, which has CAS Registry Number 97-54-1 and is described in The Merck Index monograph M6486; and the anion is 2-methoxy-4-(prop-1-enyl)phenolate.

212. The composition of any one of claims 1-19, wherein the molecule is isomaltol, which has CAS Registry Number 3420-59-5 and is described in The Merck Index monograph M6497; and the anion is 2-acetylfuran-3-oxide.

213. The composition of any one of claims 1-19, wherein the molecule is isosilybin A, which has CAS Registry Number 142796-21-2 and is described in The Merck Index monograph M9941; and the anion is selected from 3,7-dihydroxy-4-oxo-2-[3-(4-hydroxy-3-methoxyphenyl)-2-(hydroxymethyl)-2,3-dihydro-1,4-benzodioxin-7-yl]-2,3-dihydro-4H-chromene-5-oxide; 3,5-dihydroxy-4-oxo-2-[3-(4-hydroxy-3-methoxyphenyl)-2-(hydroxymethyl)-2,3-dihydro-1,4-benzodioxin-7-yl]-2,3-dihydro-4H-chromene-7-oxide; and 2-methoxy-4-[2-hydroxymethyl-7-(3,5,7-trihydroxy-4-oxo-2,3-dihydro-4H-chromen-2-yl)-2,3-dihydro-1,4-benzodioxin-3-yl]phenolate.

214. The composition of any one of claims 1-19, wherein the molecule is ivacaftor, which has CAS Registry Number 873054-44-5 and is described in The Merck Index monograph M6565; and the anion is 3-{N-[(4-oxo-1H-quinolin-3-yl)carbonyl]amino}-4,6-bis(tert-butyl)phenolate.

215. The composition of any one of claims 1-19, wherein the molecule is javanicin, which has CAS Registry Number 476-45-9 and is described in The Merck Index monograph M6580; and the anion is selected from 4-hydroxy-2-methoxy-6-methyl-5,8-dioxo-7-(2-oxopropyl)naphthalene-1-oxide and 5,8-dioxo-4-hydroxy-3-methoxy-7-methyl-6-(2-oxo-propyl)-naphthalene-1-oxide.

216. The composition of any one of claims 1-19, wherein the molecule is juglone, which has CAS Registry Number 481-39-0 and is described in The Merck Index monograph M6587; and the anion is 5,8-dioxonaphthalene-1-oxide.

217. The composition of any one of claims 1-19, wherein the molecule is kaempferol, which has CAS Registry Number 520-18-3 and is described in The Merck Index monograph M6592; and the anion is selected from 5,7-dihydroxy-4-oxo-2-(4-hydroxyphenyl)-4H-chromene-3-oxide; 3,7-dihydroxy-4-oxo-2-(4-hydroxyphenyl)-4H-chromene-5-oxide; 3,5-dihydroxy-4-oxo-2-(4-hydroxyphenyl)-4H-chromene-7-oxide; and 4-(3,5,7-trihydroxy-4-oxo-4H-chromen-2-yl)phenolate.

218. The composition of any one of claims 1-19, wherein the molecule is lapachol, which has CAS Registry Number 84-79-7 and is described in The Merck Index monograph M6687; and the anion is 3-isoprenyl-1,4-dioxonaphthalene-2-oxide.

219. The composition of any one of claims 1-19, wherein the molecule is lawsone, which has CAS Registry Number 83-72-7 and is described in The Merck Index monograph M6718; and the anion is 1,4-dioxonaphthalene-2-oxide.

220. The composition of any one of claims 1-19, wherein the molecule is leucocyanidin, which has CAS Registry Number 480-17-1 and is described in The Merck Index monograph M6774; and the anion is selected from 3,4,7-trihydroxy-2-(3,4-dihydroxyphenyl)-3,4-dihydro-2H-chromene-5-oxide; 3,4,5-trihydroxy-2-(3,4-dihydroxyphenyl)-3,4-dihydro-2H-chromene-7-oxide; 2-hydroxy-4-(3,4,5,7-tetrahydroxy-3,4-dihydro-2H-chromen-2-yl)phenolate; and 2-hydroxy-5-(3,4,5,7-tetrahydroxy-3,4-dihydro-2H-chromen-2-yl)phenolate.

221. The composition of any one of claims 1-19, wherein the molecule is licochalcone A, which has CAS Registry Number 58749-22-7 and is described in The Merck Index monograph M6802; and the anion is selected from 4-{1-oxo-3-[4-hydroxy-2-methoxy-5-(2-methylbut-3-en-2-yl)phenyl]prop-2-enyl}phenolate and 3-methoxy-6-(2-methylbut-3-en-2-yl)-4-[3-oxo-3-(4-hydroxyphenyl)prop-1-enyl]phenolate.

222. The composition of any one of claims 1-19, wherein the molecule is licochalcone B, which has CAS Registry Number 58749-23-8 and is described in The Merck Index monograph M6802; and the anion is selected from 4-[1-oxo-3-(3,4-dihydroxy-2-methoxyphenyl)prop-2-enyl]phenolate; 2-hydroxy-3-methoxy-4-[3-oxo-3-(4-hydroxyphenyl)prop-1-enyl]phenolate; and 6-hydroxy-2-methoxy-3-[3-oxo-3-(4-hydroxyphenyl)prop-1-enyl]phenolate.

223. The composition of any one of claims 1-19, wherein the molecule is licochalcone C, which has CAS Registry Number 144506-14-9 and is described in The Merck Index monograph M6802; and the anion is selected from 4-{1-oxo-3-[4-hydroxy-3-isoprenyl-2-methoxyphenyl]prop-2-enyl}phenolate and 2-isoprenyl-3-methoxy-4-[3-oxo-3-(4-hydroxyphenyl)prop-1-enyl]phenolate.

224. The composition of any one of claims 1-19, wherein the molecule is licochalcone D, which has CAS Registry Number 144506-15-0 and is described in The Merck Index monograph M6802; and the anion is selected from 2-isoprenyl-4-[1-oxo-3-(3,4-dihydroxy-2-methoxyphenyl)prop-2-enyl]phenolate; 2-hydroxy-3-methoxy-4-[3-oxo-3-(4-hydroxy-3-isoprenylphenyl)prop-1-enyl]phenolate; and 6-hydroxy-2-methoxy-3-[3-oxo-3-(4-hydroxy-3-isoprenylphenyl)prop-1-enyl]phenolate.

225. The composition of any one of claims 1-19, wherein the molecule is licochalcone E, which has CAS Registry Number 864232-34-8 and is related to The Merck Index monograph M6802; and the anion is selected from 4-{1-oxo-3-[4-hydroxy-2-methoxy-5-(3-methylbut-3-en-2-yl)phenyl]prop-2-enyl}phenolate and 3-methoxy-6-(3-methylbut-3-en-2-yl)-4-[3-oxo-3-(4-hydroxyphenyl)prop-1-enyl]phenolate.

226. The composition of any one of claims 1-19, wherein the molecule is licochalcone F, which is related to The Merck Index monograph M6802; and the anion is selected from 4-{1-oxo-3-[4-hydroxy-2-methoxy-3-(3-methylbut-3-en-2-yl)phenyl]prop-2-enyl}phenolate and 3-methoxy-2-(3-methylbut-3-en-2-yl)-4-[3-oxo-3-(4-hydroxyphenyl)prop-1-enyl]phenolate.

227. The composition of any one of claims 1-19, wherein the molecule is linomide, which has CAS Registry Number 84088-42-6 and is described in The Merck Index monograph M9660; and the anion is N-methyl-2-oxo-3-[(N-methyl-N-phenylamino)carbonyl]-1,2-dihydroquinoline-4-oxide.

228. The composition of any one of claims 1-19, wherein the molecule is luteolin, which has CAS Registry Number 491-70-3 and is described in The Merck Index monograph M6945; and the anion is selected from 7-hydroxy-4-oxo-2-(3,4-dihydroxyphenyl)-4H-chromene-5-oxide; 5-hydroxy-4-oxo-2-(3,4-dihydroxyphenyl)-4H-chromene-7-oxide; 2-hydroxy-4-(5,7-dihydroxy-4-oxo-4H-chromen-2-yl)phenolate; and 2-hydroxy-5-(5,7-dihydroxy-4-oxo-4H-chromen-2-yl)phenolate.

229. The composition of any one of claims 1-19, wherein the molecule is magnolol, which has CAS Registry Number 528-43-8 and is described in The Merck Index monograph M7031; and the anion is 4-(prop-2-enyl)-2-[2-hydroxy-5-(prop-2-enyl)phenyl]phenolate.

230. The composition of any one of claims 1-19, wherein the molecule is maltol, which has CAS Registry Number 118-71-8 and is described in The Merck Index monograph M7047; and the anion is 2-methyl-4-oxo-4H-pyran-3-oxide.

231. The composition of any one of claims 1-19, wherein the molecule is mangostin, which has CAS Registry Number 6147-11-1 and is described in The Merck Index monograph M7077; and the anion is selected from 6,8-dihydroxy-1,7-diisoprenyl-2-methoxy-9-oxoxanthene-3-oxide; 3,6-dihydroxy-2,8-diisoprenyl-7-methoxy-9-oxoxanthene-1-oxide; and 1,6-dihydroxy-2,8-diisoprenyl-7-methoxy-9-oxoxanthene-3-oxide.

232. The composition of any one of claims 1-19, wherein the molecule is medicagol, which has CAS Registry Number 1983-72-8 and is described in The Merck Index monograph M7129; and the anion is 6-oxo-(1,3)dioxolo(5,6)[1]benzofuro[3,2-c]chromene-3-oxide.

233. The composition of any one of claims 1-19, wherein the molecule is medicarpin, which has CAS Registry Number 32383-76-9 and is described in The Merck Index monograph M7130; and the anion is 9-methoxy-6a,11a-dihydro-6H-[1]benzofuro[3,2-c]chromene-3-oxide.

234. The composition of any one of claims 1-19, wherein the molecule is menadiol, which has CAS Registry Number 481-85-6 and is described in The Merck Index monograph M7168; and the anion is selected from 4-hydroxy-2-methylnaphthalene-1-oxide and 4-hydroxy-3-methyl-naphthalene-1-oxide.

235. The composition of any one of claims 1-19, wherein the molecule is mestilbol, which has CAS Registry Number 18839-90-2 and is described in The Merck Index monograph M4418; and the anion is 4-[4-(4-methoxyphenyl)hex-3-en-3-yl]phenolate.

236. The composition of any one of claims 1-19, wherein the molecule is meta-cresol, which has CAS Registry Number 108-39-4 and is described in The Merck Index monograph M3834; and the anion is 3-methylphenolate.

237. The composition of any one of claims 1-19, wherein the molecule is methestrol, which has CAS Registry Number 130-73-4 and is described in The Merck Index monograph M7310; and the anion is 2-methyl-4-[4-(4-hydroxy-3-methylphenyl)hex-3-yl]phenolate.

238. The composition of any one of claims 1-19, wherein the molecule is methylchrysin, which has CAS Registry Number 18651-45-1 and is described in The Merck Index monograph M3530; and the anion is selected from 5-hydroxy-3-methyl-4-oxo-2-phenyl-4H-chromene-7-oxide and 7-hydroxy-3-methyl-4-oxo-2-phenyl-4H-chromene-5-oxide.

239. The composition of any one of claims 1-19, wherein the molecule is methylparaben, which has CAS Registry Number 99-76-3 and is described in The Merck Index monograph M7450; and the anion is 4-[(methoxy)carbonyl]phenolate.

240. The composition of any one of claims 1-19, wherein the molecule is mexenone, which has CAS Registry Number 1641-17-4 and is described in The Merck Index monograph M7516; and the anion is 3-methoxy-6-[(4-methylphenyl)carbonyl]phenolate.

241. The composition of any one of claims 1-19, wherein the molecule is monobenzone, which has CAS Registry Number 103-16-2 and is described in The Merck Index monograph M7604; and the anion is 4-[(benzyl)oxy]phenolate.

242. The composition of any one of claims 1-19, wherein the molecule is morin, which has CAS Registry Number 480-16-0 and is described in The Merck Index monograph M7625; and the anion is selected from 5,7-dihydroxy-4-oxo-2-(2,4-dihydroxyphenyl)-4H-chromene-3-oxide; 3,7-dihydroxy-4-oxo-2-(2,4-dihydroxyphenyl)-4H-chromene-5-oxide; 3,5-dihydroxy-4-oxo-2-(2,4-dihydroxyphenyl)-4H-chromene-7-oxide; 3-hydroxy-4-(3,5,7-trihydroxy-4-oxo-4H-chromen-2-yl)phenolate; and 3-hydroxy-6-(3,5,7-trihydroxy-4-oxo-4H-chromen-2-yl)phenolate.

243. The composition of any one of claims 1-19, wherein the molecule is morphenol, which has CAS Registry Number 519-56-2 and is described in The Merck Index monograph M834; and the anion is phenanthro[4,5-bcd]furan-3-oxide.

244. The composition of any one of claims 1-19, wherein the molecule is moxestrol, which has CAS Registry Number 34816-55-2 and is described in The Merck Index monograph M7645; and the anion is 17-ethynyl-17-hydroxy-11-methoxy-13-methyl-7,8,9,11,12,14,15,16-octahydro-6H-cyclopenta[a]phenanthrene-3-oxide.

245. The composition of any one of claims 1-19, wherein the molecule is myricetin, which has CAS Registry Number 529-44-2 and is described in The Merck Index monograph M7687; and the anion is selected from 5,7-dihydroxy-4-oxo-2-(3,4,5-trihydroxyphenyl)-4H-chromene-3-oxide; 3,7-dihydroxy-4-oxo-2-(3,4,5-trihydroxyphenyl)-4H-chromene-5-oxide; 3,5-dihydroxy-4-oxo-2-(3,4,5-trihydroxyphenyl)-4H-chromene-7-oxide; 2,6-dihydroxy-4-(3,5,7-trihydroxy-4-oxo-4H-chromen-2-yl)phenolate; and 2,3-dihydroxy-5-(3,5,7-trihydroxy-4-oxo-4H-chromen-2-yl)phenolate.

246. The composition of any one of claims 1-19, wherein the molecule is nabilone, which has CAS Registry Number 51022-71-0 and is described in The Merck Index monograph M7697; and the anion is 6,6-dimethyl-9-oxo-3-(2-methyloct-2-yl)-7,8,10,10a-tetrahydro-6aH-benzo[c]chromene-1-oxide.

247. The composition of any one of claims 1-19, wherein the molecule is naphthoresorcinol, which has CAS Registry Number 132-86-5 and is described in The Merck Index monograph M7751; and the anion is selected from 3-hydroxynapthalene-1-oxide and 4-hydroxynapthalene-2-oxide.

248. The composition of any one of claims 1-19, wherein the molecule is naringenin, which has CAS Registry Number 480-41-1 and is described in The Merck Index monograph M7776; and the anion is selected from 7-hydroxy-4-oxo-2-(4-hydroxyphenyl)-2,3-dihydro-4H-chromene-5-oxide; 5-hydroxy-4-oxo-2-(4-hydroxyphenyl)-2,3-dihydro-4H-chromene-7-oxide; and 4-(5,7-dihydroxy-4-oxo-2,3-dihydro-4H-chromen-2-yl)phenolate.

249. The composition of any one of claims 1-19, wherein the molecule is nimbiol, which has CAS Registry Number 561-95-5 and is described in The Merck Index monograph M7902; and the anion is 2,4b,8,8-tetramethyl-10-oxo-4b,5,6,7,8a,9-hexahydrophenanthrene-3-oxide.

250. The composition of any one of claims 1-19, wherein the molecule is nonivamide, which has CAS Registry Number 2444-46-4 and is related to The Merck Index monograph M3040; and the anion is 2-methoxy-4-{[N-(1-oxononyl)amine]methyl}phenolate.

251. The composition of any one of claims 1-19, wherein the molecule is nordihydrocapsaicin, which has CAS Registry Number 28789-35-7 and is related to The Merck Index monograph M3040; and the anion is 2-methoxy-4-{[N-(7-methyl-1-oxooctyl)amino]methyl}phenolate.

252. The composition of any one of claims 1-19, wherein the molecule is nordihydroguaiaretic acid, which has CAS Registry Number 500-38-9 and is described in The Merck Index monograph M8052; and the anion is selected from 2-hydroxy-4-[2,3-dimethyl-4-(3,4-dihydroxyphenyl)butyl]phenolate and 2-hydroxy-5-[2,3-dimethyl-4-(3,4-dihydroxyphenyl)butyl]phenolate.

253. The composition of any one of claims 1-19, wherein the molecule is octabenzone, which has CAS Registry Number 1843-05-6 and is described in The Merck Index monograph M8101; and the anion is 6-[(phenyl)carbonyl]-3-[(octyl)oxy]phenolate.

254. The composition of any one of claims 1-19, wherein the molecule is oleocanthal, which has CAS Registry Number 289030-99-5 and is described in The Merck Index monograph M8192; and the anion is 4-(2-{[3-(2-oxoethyl)-4-formyl-1-oxohex-4-enyl]oxy}ethyl)phenolate.

255. The composition of any one of claims 1-19, wherein the molecule is oroxylin A, which has CAS Registry Number 480-11-5 and is described in The Merck Index monograph M8244; and the anion is selected from 7-hydroxy-6-methoxy-4-oxo-2-phenyl-4H-chromene-5-oxide and 5-hydroxy-6-methoxy-4-oxo-2-phenyl-4H-chromene-7-oxide.

256. The composition of any one of claims 1-19, wherein the molecule is ortho-benzylphenol, which has CAS Registry Number 28994-41-4 and is described in The Merck Index monograph M2414; and the anion is 2-benzylphenolate.

257. The composition of any one of claims 1-19, wherein the molecule is orthocaine, which has CAS Registry Number 536-25-4 and is described in The Merck Index monograph M8249; and the anion is 2-amino-4-(methoxycarbonyl)phenolate.

258. The composition of any one of claims 1-19, wherein the molecule is ortho-cresol, which has CAS Registry Number 95-48-7 and is described in The Merck Index monograph M3834; and the anion is 2-methylphenolate.

259. The composition of any one of claims 1-19, wherein the molecule is oryzanol A, which has CAS Registry Number 11042-64-1 and is described in The Merck Index monograph M8253; and the anion is 2-methoxy-4-(3-oxo-3-{[4,4,13,14-tetramethyl-9,10-methanediyl-17-(6-methylhept-5-en-2-yl)-1,2,3,5,6,7,8,11,12,15,16,17-dodecahydrocyclopenta[a]phenanthren-3-yl]oxy}prop-1-enyl)phenolate.

260. The composition of any one of claims 1-19, wherein the molecule is oryzanol C, which has CAS Registry Number 469-36-3 and is described in The Merck Index monograph M8253; and the anion is 2-methoxy-4-(3-oxo-3-{[4,4,13,14-tetramethyl-9,10-methanediyl-17-(5-methylidene-6-methylhept-2-yl)-1,2,3,5,6,7,8,11,12,15,16,17-dodecahydrocyclopenta[a]phenanthren-3-yl]oxy}prop-1-enyl)phenolate.

261. The composition of any one of claims 1-19, wherein the molecule is osajin, which has CAS Registry Number 482-53-1 and is described in The Merck Index monograph M860; and the anion is selected from 6-isoprenyl-8,8-dimethyl-4-oxo-3-(4-hydroxyphenyl)pyrano[2,3-h]chromene-5-oxide and 4-(5-hydroxy-6-isoprenyl-8,8-dimethyl-4-oxopyrano[2,3-h]chromen-3-yl)phenolate.

262. The composition of any one of claims 1-19, wherein the molecule is osalmid, which has CAS Registry Number 526-18-1 and is described in The Merck Index monograph M8254; and the anion is selected from 2-[N-(4-hydroxyphenyl)carbamoyl]phenolate and 4-{N-[(2-hydroxyphenyl)carbonyl]amino}phenolate.

263. The composition of any one of claims 1-19, wherein the molecule is ostruthin, which has CAS Registry Number 148-83-4 and is described in The Merck Index monograph M8264; and the anion is 6-geranyl-2-oxo-2H-chromene-7-oxide.

264. The composition of any one of claims 1-19, wherein the molecule is ourateacatechin, which has CAS Registry Number 17291-05-3 and is related to The Merck Index monograph M3172; and the anion is selected from 3,5-dihydroxy-2-(3,5-dihydroxy-4-methoxyphenyl)-3,4-dihydro-2H-chromene-7-oxide; 3,7-dihydroxy-2-(3,5-dihydroxy-4-methoxyphenyl)-3,4-dihydro-2H-chromene-5-oxide; and 3-hydroxy-2-methoxy-5-(3,5,7-trihydroxy-3,4-dihydro-2H-chromen-2-yl)phenolate.

265. The composition of any one of claims 1-19, wherein the molecule is oxabolone, which has CAS Registry Number 4721-69-1 and is described in The Merck Index monograph M379; and the anion is (8R,9S,10R,13S,14S,17S)-17-hydroxy-13-methyl-3-oxo-2,6,7,8,9,10,11,12,14,15,16,17-dodecahydro-1H-cyclopenta[a]phenanthrene-4-oxide.

266. The composition of any one of claims 1-19, wherein the molecule is oxabolone 17-cyclopentanepropionate, which has CAS Registry Number 1254-35-9 and is described in The Merck Index monograph M379; and the anion is (8R,9S,10R,13S,14S,17S)-13-methyl-3-oxo-17-[(3-cyclopentyl-1-oxopropyl)oxy]-2,6,7,8,9,10,11,12,14,15,16,17-dodecahydro-1H-cyclopenta[a]phenanthrene-4-oxide.

267. The composition of any one of claims 1-19, wherein the molecule is oxybenzone, which has CAS Registry Number 131-57-7 and is described in The Merck Index monograph M8323; and the anion is 3-methoxy-6-[(phenyl)carbonyl]phenolate.

268. The composition of any one of claims 1-19, wherein the molecule is oxymesterone, which has CAS Registry Number 145-12-0 and is described in The Merck Index monograph M8334; and the anion is 17-hydroxy-10,13,17-trimethyl-3-oxo-2,6,7,8,9,11,12,14,15,16-decahydro-1H-cyclopenta[a]phenanthrene-4-oxide.

269. The composition of any one of claims 1-19, wherein the molecule is oxymetholone, which has CAS Registry Number 434-07-1 and is described in The Merck Index monograph M8336; and the anion is [17-hydroxy-10,13,17-trimethyl-3-oxo-1,4,5,6,7,8,9,11,12,14,15,16-dodecahydrocyclopenta[a]phenanthren-2-ylidene]methane-oxide.

270. The composition of any one of claims 1-19, wherein the molecule is oxyphenbutazone, which has CAS Registry Number 129-20-4 and is described in The Merck Index monograph M8341; and the anion is 4-(4-butyl-3,5-dioxo-2-phenylpyrazolidin-1-yl)phenolate.

271. The composition of any one of claims 1-19, wherein the molecule is para-anol, which has CAS Registry Number 539-12-8 and is described in The Merck Index monograph M1940; and the anion is 4-(prop-1-enyl)phenolate.

272. The composition of any one of claims 1-19, wherein the molecule is para-benzylphenol, which has CAS Registry Number 101-53-1 and is described in The Merck Index monograph M2415; and the anion is 4-benzylphenolate.

273. The composition of any one of claims 1-19, wherein the molecule is para-cresol, which has CAS Registry Number 106-44-5 and is described in The Merck Index monograph M3834; and the anion is 4-methylphenolate.

274. The composition of any one of claims 1-19, wherein the molecule is para-tert-pentylphenol, which has CAS Registry Number 80-46-6 and is described in The Merck Index monograph M8525; and the anion is 4-(tert-pentyl)phenolate.

275. The composition of any one of claims 1-19, wherein the molecule is para-vinylguaiacol, which has CAS Registry Number 7786-61-0 and is related to The Merck Index monograph M5859; and the anion is 4-ethenyl-2-methoxyphenolate.

276. The composition of any one of claims 1-19, wherein the molecule is parietin, which has CAS Registry Number 521-61-9 and is described in The Merck Index monograph M4888; and the anion is selected from 8-hydroxy-6-methyl-3-methoxy-9,10-dioxoanthracene-1-oxide and 8-hydroxy-3-methyl-6-methoxy-9,10-dioxoanthracene-1-oxide.

277. The composition of any one of claims 1-19, wherein the molecule is paroxypropione, which has CAS Registry Number 70-70-2 and is described in The Merck Index monograph M8419; and the anion is 4-(1-oxopropyl)phenolate.

278. The composition of any one of claims 1-19, wherein the molecule is parvaquone, which has CAS Registry Number 4042-30-2 and is described in The Merck Index monograph M8424; and the anion is 3-cyclohexyl-1,4-dioxonaphthalene-2-oxide.

279. The composition of any one of claims 1-19, wherein the molecule is pectolinarigenin, which has CAS Registry Number 520-12-7 and is described in The Merck Index monograph M8440; and the anion is selected from 7-hydroxy-6-methoxy-4-oxo-2-(4-methoxyphenyl)-4H-chromene-5-oxide and 5-hydroxy-6-methoxy-4-oxo-2-(4-methoxyphenyl)-4H-chromene-7-oxide.

280. The composition of any one of claims 1-19, wherein the molecule is perezone, which has CAS Registry Number 3600-95-1 and is described in The Merck Index monograph M8539; and the anion is 6-methyl-3-(6-methylhept-5-en-2-yl)-1,4-benzoquinone-2-oxide.

281. The composition of any one of claims 1-19, wherein the molecule is phaseolin, which has CAS Registry Number 13401-40-6 and is described in The Merck Index monograph M8583; and the anion is 3,3-dimethyl-7,12b-dihydro-6bH-furo[3,2-c:5,4-f]dichromen-10-oxide.

282. The composition of any one of claims 1-19, wherein the molecule is phenolphthalol, which has CAS Registry Number 81-92-5 and is described in The Merck Index monograph M8628; and the anion is 4-{(4-hydroxyphenyl)-[2-(hydroxymethyl)phenyl]methyl}phenolate.

283. The composition of any one of claims 1-19, wherein the molecule is phenprocoumon, which has CAS Registry Number 435-97-2 and is described in The Merck Index monograph M8641; and the anion is 2-oxo-3-(1-phenylpropyl)-2H-chromene-4-oxide.

284. The composition of any one of claims 1-19, wherein the molecule is phloretin, which has CAS Registry Number 60-82-2 and is described in The Merck Index monograph M8707; and the anion is selected from 3,5-dihydroxy-2-[1-oxo-3-(4-hydroxyphenyl)propyl]phenolate; 3,5-dihydroxy-4-[1-oxo-3-(4-hydroxyphenyl)propyl]phenolate; and 4-[3-oxo-3-(2,4,6-trihydroxyphenyl)propyl]phenolate.

285. The composition of any one of claims 1-19, wherein the molecule is phthiocol, which has CAS Registry Number 483-55-6 and is described in The Merck Index monograph M8760; and the anion is 3-methyl-1,4-dioxonaphthalene-2-oxide.

286. The composition of any one of claims 1-19, wherein the molecule is pinosylvin, which has CAS Registry Number 22139-77-1 and is described in The Merck Index monograph M8833; and the anion is 3-hydroxy-5-(2-phenylethenyl)phenolate.

287. The composition of any one of claims 1-19, wherein the molecule is pinosylvin monomethyl ether, which has CAS Registry Number 35302-70-6 and is described in The Merck Index monograph M8833; and the anion is 3-methoxy-5-(2-phenylethenyl)phenolate.

288. The composition of any one of claims 1-19, wherein the molecule is plumbagin, which has CAS Registry Number 481-42-5 and is described in The Merck Index monograph M8925; and the anion is 6-methyl-5,8-dioxonaphthalene-1-oxide.

289. The composition of any one of claims 1-19, wherein the molecule is pratensein, which has CAS Registry Number 2284-31-3 and is described in The Merck Index monograph M9104; and the anion is selected from 7-hydroxy-4-oxo-3-(3-hydroxy-4-methoxyphenyl)-4H-chromene-5-oxide; 5-hydroxy-4-oxo-3-(3-hydroxy-4-methoxyphenyl)-4H-chromene-7-oxide; and 2-methoxy-5-(5,7-dihydroxy-4-oxo-4H-chromen-3-yl)phenolate.

290. The composition of any one of claims 1-19, wherein the molecule is probucol, which has CAS Registry Number 23288-49-5 and is described in The Merck Index monograph M9143; and the anion is 2,6-di-tert-butyl-4-({2-[(3,5-di-tert-butyl-4-hydroxyphenyl)sulfanyl]prop-2-yl}sulfanyl)phenolate.

291. The composition of any one of claims 1-19, wherein the molecule is procerin, which has CAS Registry Number 552-96-5 and is described in The Merck Index monograph M434; and the anion is 4-isoprenyl-5-isopropenyl-7-oxocyclohepta-1,3,5-trien-1-oxide.

292. The composition of any one of claims 1-19, wherein the molecule is propofol, which has CAS Registry Number 2078-54-8 and is described in The Merck Index monograph M9217; and the anion is 2,6-diisopropylphenolate.

293. The composition of any one of claims 1-19, wherein the molecule is propyl gallate, which has CAS Registry Number 121-79-9 and is described in The Merck Index monograph M9241; and the anion is selected from 2,3-dihydroxy-5-[(propoxy)carbonyl]phenolate and 2,6-dihydroxy-4-[(propoxy)carbonyl]phenolate.

294. The composition of any one of claims 1-19, wherein the molecule is propylparaben, which has CAS Registry Number 94-13-3 and is described in The Merck Index monograph M9247; and the anion is 4-[(propoxy)carbonyl]phenolate.

295. The composition of any one of claims 1-19, wherein the molecule is protiofate, which has CAS Registry Number 58416-00-5 and is described in The Merck Index monograph M1243; and the anion is 4-hydroxy-2,5-bis{[(propyl)oxy]carbonyl}thiophene-3-oxide.

296. The composition of any one of claims 1-19, wherein the molecule is protocatechualdehyde, which has CAS Registry Number 139-85-5 and is described in The Merck Index monograph M9272; and the anion is selected from 4-formyl-2-hydroxyphenolate and 5-formyl-2-hydroxyphenolate.

297. The composition of any one of claims 1-19, wherein the molecule is prunetin, which has CAS Registry Number 552-59-0 and is described in The Merck Index monograph M9287; and the anion is selected from 7-methoxy-4-oxo-3-(4-hydroxyphenyl)-4H-chromene-5-oxide and 4-(5-hydroxy-7-methoxy-4-oxo-4H-chromen-3-yl)phenolate.

298. The composition of any one of claims 1-19, wherein the molecule is pseudobaptigenin, which has CAS Registry Number 90-29-9 and is described in The Merck Index monograph M9289; and the anion is 3-(1,3-benzodioxol-5-yl)-4-oxo-4H-chromene-7-oxide.

299. The composition of any one of claims 1-19, wherein the molecule is psiguadial A, which has CAS Registry Number 1254173-25-5 and is described in The Merck Index monograph M11799; and the anion is selected from 4,6-diformyl-7-hydroxy-10,13,13,17-tetramethyl-9-phenyl-2-oxapentacyclo[8.7.2.0^{1,11}.0^{3,8}.0^{12,14}]nonadeca-3,5,7-triene-5-oxide and 4,6-diformyl-5-hydroxy-10,13,13,17-tetramethyl-9-phenyl-2-oxapentacyclo[8.7.2.0^{1,11}.0^{3,8}.0^{12,14}]nonadeca-3,5,7-triene-7-oxide.

300. The composition of any one of claims 1-19, wherein the molecule is psiguadial B, which is related to The Merck Index monograph M11799; and the anion is selected from 4,6-diformyl-7-hydroxy-13,16,16-trimethyl-9-phenyl-2-oxapentacyclo[11.6.1.0^{1,1}.0^{3,8}.0^{14,17}]icosa-3,5,7-triene-5-oxide and 4,6-diformyl-5-hydroxy-13,16,16-trimethyl-9-phenyl-2-oxapentacyclo[11.6.1.06.010014]icosa-3,5,7-triene-7-oxide.

301. The composition of any one of claims 1-19, wherein the molecule is psiguadial C, which is related to The Merck Index monograph M11799; and the anion is selected from 4,6-diformyl-7-hydroxy-10,14,19,19-tetramethyl-9-phenyl-2,13-dioxapentacyclo[8.10.0.0^{3,8}.0^{12,14}.0^{18,20}]icosa-3,5,7-triene-5-oxide and 4,6-diformyl-5-hydroxy-10,14,19,19-tetramethyl-9-phenyl-2,13-dioxapentacyclo[8.10.0.0^{3,8}.0^{12,14}.0^{18,20}]icosa-3,5,7-triene-7-oxide.

302. The composition of any one of claims 1-19, wherein the molecule is psiguadial D, which has CAS Registry Number 1402066-95-8 and is related to The Merck Index monograph M11799; and the anion is selected from 4,6-diformyl-7-hydroxy-10,14,18,18-tetramethyl-9-phenyl-2-oxa-tetracyclo[8.10.0.0^{3,8}.0^{17,19}]nonadeca-3,5,7,13-tetraene-5-oxide and 4,6-diformyl-5-hydroxy-10,14,18,18-tetramethyl-9-phenyl-2-oxa-tetracyclo[8.10.0.0^{3,8}.0^{17,19}]nonadeca-3,5,7,13-tetraene-7-oxide.

303. The composition of any one of claims 1-19, wherein the molecule is psoralidin, which has CAS Registry Number 18642-23-4 and is described in The Merck Index monograph M442; and the anion is selected from 9-hydroxy-2-isoprenyl-6-oxo-[1]benzofurano[3,2-c]chromene-3-oxide and 3-hydroxy-2-isoprenyl-6-oxo-[1]benzofurano[3,2-c]chromen-9-oxide.

304. The composition of any one of claims 1-19, wherein the molecule is purpurin, which has CAS Registry Number 81-54-9 and is described in The Merck Index monograph M9326; and the anion is selected from 2,4-dihydroxy-9,10-dioxoanthracene-1-oxide; 1,4-dihydroxy-9,10-dioxoanthracene-2-oxide; and 3,4-dihydroxy-9,10-dioxoanthracene-1-oxide.

305. The composition of any one of claims 1-19, wherein the molecule is purpurin 1-methyl ether, which has CAS Registry Number 94099-66-8 and is described in The Merck Index monograph M9326; and the anion is selected from 3-hydroxy-4-methoxy-9,10-dioxoanthracene-1-oxide and 4-hydroxy-1-methoxy-9,10-dioxoanthracene-2-oxide.

306. The composition of any one of claims 1-19, wherein the molecule is purpurin 2,4-dimethyl ether, which is described in The Merck Index monograph M9326; and the anion is 2,4-dimethoxy-9,10-dioxoanthracene-1-oxide.

307. The composition of any one of claims 1-19, wherein the molecule is purpurin 2-methyl ether, which is described in The Merck Index monograph M9326; and the anion is selected from 4-hydroxy-2-methoxy-9,10-dioxoanthracene-1-oxide and 4-hydroxy-3-methoxy-9,10-dioxoanthracene-1-oxide.

308. The composition of any one of claims 1-19, wherein the molecule is purpurogallin, which has CAS Registry Number 569-77-7 and is described in The Merck Index monograph M9327; and the anion is selected from 3,4,6-trihydroxy-5-oxobenzo[7]annulen-2-oxide; 1,3,8-trihydroxy-9-oxobenzo[7]annulen-2-oxide; 2,3,8-trihydroxy-9-oxobenzo[7]annulen-1-oxide; and 2,3,4-trihydroxy-5-oxobenzo[7]annulen-6-oxide.

309. The composition of any one of claims 1-19, wherein the molecule is quercetagetin, which has CAS Registry Number 90-18-6 and is described in The Merck Index monograph M9419; and the anion is selected from 5,6,7-trihydroxy-4-oxo-2-(3,4-dihydroxyphenyl)-4H-chromene-3-oxide; 3,6,7-trihydroxy-4-oxo-2-(3,4-dihydroxyphenyl)-4H-chromene-5-oxide; 3,5,7-trihydroxy-4-oxo-2-(3,4-dihydroxyphenyl)-4H-chromene-6-oxide; 3,5,6-trihydroxy-4-oxo-2-(3,4-dihydroxyphenyl)-4H-chromene-7-oxide; 2-hydroxy-4-(3,5,6,7-tetrahydroxy-4-oxo-4H-chromen-2-yl)phenolate; and 2-hydroxy-5-(3,5,6,7-tetrahydroxy-4-oxo-4H-chromen-2-yl)phenolate.

310. The composition of any one of claims 1-19, wherein the molecule is quercetin, which has CAS Registry Number 117-39-5 and is described in The Merck Index monograph M9420; and the anion is selected from 5,7-dihydroxy-4-oxo-2-(3,4-dihydroxyphenyl)-4H-chromene-3-oxide; 3,7-dihydroxy-4-oxo-2-(3,4-dihydroxyphenyl)-4H-chromene-5-oxide; 3,5-dihydroxy-4-oxo-2-(3,4-dihydroxyphenyl)-4H-chromene-7-oxide; 2-hydroxy-4-(3,5,7-trihydroxy-4-oxo-4H-chromen-2-yl)phenolate; and 2-hydroxy-5-(3,5,7-trihydroxy-4-oxo-4H-chromen-2-yl)phenolate.

311. The composition of any one of claims 1-19, wherein the molecule is quinalizarin, which has CAS Registry Number 81-61-8 and is described in The Merck Index monograph M9436; and the anion is selected from 2,5,8-trihydroxy-9,10-dioxoanthracene-1-oxide; 1,5,8-trihydroxy-9,10-dioxoanthracene-2-oxide; 4,5,6-trihydroxy-9,10-dioxoanthracene-1-oxide; and 4,7,8-trihydroxy-9,10-dioxoanthracene-1-oxide.

312. The composition of any one of claims 1-19, wherein the molecule is raspberry ketone, which has CAS Registry Number 5471-51-2 and is described in The Merck Index monograph M11999; and the anion is 4-(3-oxobutyl)phenolate.

313. The composition of any one of claims 1-19, wherein the molecule is resacetophenone, which has CAS Registry Number 89-84-9 and is described in The Merck Index monograph M9530; and the anion is selected from 4-acetyl-3-hydroxyphenolate and 6-acetyl-3-hydroxyphenolate.

314. The composition of any one of claims 1-19, wherein the molecule is resveratrol, which has CAS Registry Number 501-36-0 and is described in The Merck Index monograph M9549; and the anion is selected from 4-[2-(3,5-dihydroxyphenyl)ethenyl]phenolate and 3-hydroxy-5-[2-(4-hydroxyphenyl)ethenyl]phenolate.

315. The composition of any one of claims 1-19, wherein the molecule is rhamnetin, which has CAS Registry Number 90-19-7 and is described in The Merck Index monograph M9564; and the anion is selected from 5-hydroxy-7-methoxy-4-oxo-2-(3,4-hydroxyphenyl)-4H-chromene-3-oxide; 3-hydroxy-7-methoxy-4-oxo-2-(3,4-hydroxyphenyl)-4H-chromene-5-oxide; 2-hydroxy-4-(3,5-dihydroxy-7-methoxy-4-oxo-4H-chromen-2-yl)phenolate; and 2-hydroxy-5-(3,5-dihydroxy-7-methoxy-4-oxo-4H-chromen-2-yl)phenolate.

316. The composition of any one of claims 1-19, wherein the molecule is rottlerin, which has CAS Registry Number 82-08-6 and is described in The Merck Index monograph M9675; and the anion is selected from 7-hydroxy-2,2-dimethyl-8-(1-oxo-3-phenylprop-2-enyl)-6-[(3-acetyl-2,4,6-trihydroxy-5-methylphenyl)methyl]-2H-chromene-5-oxide; 5-hydroxy-2,2-dimethyl-8-(1-oxo-3-phenylprop-2-enyl)-6-[(3-acetyl-2,4,6-trihydroxy-5-methylphenyl)methyl]-2H-chromene-7-oxide; 2-acetyl-3,5-dihydroxy-4-methyl-6-{[5,7-dihydroxy-2,2-dimethyl-8-(1-oxo-3-phenylprop-2-enyl)-2H-chromen-6-yl]methyl}phenolate; 4-acetyl-3,5-dihydroxy-6-methyl-2-{[5,7-dihydroxy-2,2-dimethyl-8-(1-oxo-3-phenylprop-2-enyl)-2H-chromen-6-yl]methyl}phenolate; and 2-acetyl-3,5-dihydroxy-6-methyl-4-{[5,7-dihydroxy-2,2-dimethyl-8-(1-oxo-3-phenylprop-2-enyl)-2H-chromen-6-yl]methyl}phenolate.

317. The composition of any one of claims 1-19, wherein the molecule is rottlerin 5,7-dimethyl ether, which is described in The Merck Index monograph M9675; and the anion is selected from 2-acetyl-3,5-dihydroxy-4-methyl-6-{[5,7-dimethoxy-2,2-dimethyl-8-(1-oxo-3-phenylprop-2-enyl)-2H-chromen-6-yl]methyl}phenolate; 2-acetyl-3,5-dihydroxy-6-methyl-4-{[5,7-dimethoxy-2,2-dimethyl-8-(1-oxo-3-phenylprop-2-enyl)-2H-chromen-6-yl]methyl}phenolate; and 4-acetyl-3,5-dihydroxy-6-methyl-2-{[5,7-dimethoxy-2,2-dimethyl-8-(1-oxo-3-phenylprop-2-enyl)-2H-chromen-6-yl]methyl}phenolate.

318. The composition of any one of claims 1-19, wherein the molecule is rubiadin, which has CAS Registry Number 117-02-2 and is described in The Merck Index monograph M9684; and the anion is selected from 3-hydroxy-2-methyl-9,10-dioxoanthracene-1-oxide and 4-hydroxy-3-methyl-9,10-dioxoanthracene-2-oxide.

319. The composition of any one of claims 1-19, wherein the molecule is salicyl alcohol, which has CAS Registry Number 90-01-7 and is described in The Merck Index monograph M9731; and the anion is 2-(hydroxymethyl)phenolate.

320. The composition of any one of claims 1-19, wherein the molecule is salicylaldehyde, which has CAS Registry Number 90-02-8 and is described in The Merck Index monograph M9732; and the anion is 2-formylphenolate.

321. The composition of any one of claims 1-19, wherein the molecule is salicylanilide, which has CAS Registry Number 87-17-2 and is described in The Merck Index monograph M9737; and the anion is 2-[(N-phenylamino)carbonyl]phenolate.

322. The composition of any one of claims 1-19, wherein the molecule is scopoletin, which has CAS Registry Number 92-61-5 and is described in The Merck Index monograph M9815; and the anion is 6-methoxy-2-oxo-2H-chromene-7-oxide.

323. The composition of any one of claims 1-19, wherein the molecule is scutellarein, which has CAS Registry Number 529-53-3 and is described in The Merck Index monograph M9819; and the anion is selected from 6,7-dihydroxy-4-oxo-2-(4-hydroxyphenyl)-4H-chromene-5-oxide; 5,7-dihydroxy-4-oxo-2-(4-hydroxyphenyl)-4H-chromene-6-oxide; 5,6-dihydroxy-4-oxo-2-(4-hydroxyphenyl)-4H-chromene-7-oxide; and 4-(5,6,7-trihydroxy-4-oxo-4H-chromen-2-yl)phenolate.

324. The composition of any one of claims 1-19, wherein the molecule is siccanin, which has CAS Registry Number 22733-60-4 and is described in The Merck Index monograph M9895; and the anion is 8,12,16,16-tetramethyl-3,11-dioxapentacyclo[10.7.1.0^{1,15}.0^{4,20}.0^{5,10}]icosa-5,7,9-triene-6-oxide.

325. The composition of any one of claims 1-19, wherein the molecule is silybin A, which has CAS Registry Number 22888-70-6 and is described in The Merck Index monograph M9941; and the anion is selected from 3,7-dihydroxy-4-oxo-2-[3-(4-hydroxy-3-methoxyphenyl)-2-(hydroxymethyl)-2,3-dihydro-1,4-benzodioxin-6-yl]-2,3-dihydro-4H-chromene-5-oxide; 3,5-dihydroxy-4-oxo-2-[3-(4-hydroxy-3-methoxyphenyl)-2-(hydroxymethyl)-2,3-dihydro-1,4-benzodioxin-6-yl]-2,3-dihydro-4H-chromene-7-oxide; and 2-methoxy-4-[2-hydroxymethyl-6-(3,5,7-trihydroxy-4-oxo-2,3-dihydro-4H-chromen-2-yl)-2,3-dihydro-1,4-benzodioxin-3-yl]phenolate.

326. The composition of any one of claims 1-19, wherein the molecule is sparassol, which has CAS Registry Number 520-43-4 and is described in The Merck Index monograph M10131; and the anion is 3-methoxy-6-methoxycarbonyl-5-methylphenolate.

327. The composition of any one of claims 1-19, wherein the molecule is stilbestrol, which has CAS Registry Number 659-22-3 and is described in The Merck Index monograph M4418; and the anion is 4-[2-(4-hydroxyphenyl)ethenyl]phenolate.

328. The composition of any one of claims 1-19, wherein the molecule is strobilurin F, which has CAS Registry Number 129145-63-7 and is described in The Merck Index monograph M10234; and the anion is 2-[(isoprenyl)oxy]-5-{6-methoxy-5-[(methoxy)carbonyl]-4-methylhexa-1,3,5-trienyl}phenolate.

329. The composition of any one of claims 1-19, wherein the molecule is sulfuretin, which has CAS Registry Number 120-05-8 and is described in The Merck Index monograph M10372; and the anion is selected from 3-oxo-2-[(3,4-dihydroxyphenyl)methylidene]-1-benzofuran-6-oxide; 2-hydroxy-4-[(6-hydroxy-3-oxo-1-benzofuran-2-ylidene)methyl]phenolate; and 2-hydroxy-5-[(6-hydroxy-3-oxo-1-benzofuran-2-ylidene)methyl]phenolate.

330. The composition of any one of claims 1-19, wherein the molecule is sumatrol, which has CAS Registry Number 82-10-0 and is described in The Merck Index monograph M488; and the anion is 2-isopropenyl-8,9-dimethoxy-6-oxo-2,6a,12,12a-tetrahydro-1H-chromeno[3,4-b]furo[2,3-h]chromene-5-oxide.

331. The composition of any one of claims 1-19, wherein the molecule is synhexyl, which has CAS Registry Number 117-51-1 and is described in The Merck Index monograph M10413; and the anion is 3-hexyl-6,6,9-trimethyl-7,8,9,10-tetrahydro-6H-benzo[c]chromene-1-oxide.

332. The composition of any one of claims 1-19, wherein the molecule is syringaldehyde, which has CAS Registry Number 134-96-3 and is described in The Merck Index monograph M10415; and the anion is 4-formyl-2,6-dimethoxyphenolate.

333. The composition of any one of claims 1-19, wherein the molecule is taxodione, which has CAS Registry Number 19026-31-4 and is described in The Merck Index monograph M10486; and the anion is 2-isopropyl-4b,8,8-trimethyl-3,9-dioxo-5,6,7,8a-tetrahydrophenanthrene-4-oxide.

334. The composition of any one of claims 1-19, wherein the molecule is tectorigenin, which has CAS Registry Number 548-77-6 and is described in The Merck Index monograph M10515; and the anion is selected from 7-hydroxy-6-methoxy-4-oxo-3-(4-hydroxyphenyl)-4H-chromene-5-oxide; 5-hydroxy-6-methoxy-4-oxo-3-(4-hydroxyphenyl)-4H-chromene-7-oxide; and 4-(5,7-dihydroxy-6-methoxy-4-oxo-4H-chromen-3-yl)phenolate.

335. The composition of any one of claims 1-19, wherein the molecule is tetrahydrocannabinol, which has CAS Registry Number 1972-08-3 and is described in The Merck Index monograph M10624; and the anion is 6,6,9-trimethyl-3-pentyl-6a,7,8,10a-tetrahydro-6H-benzo[c]chromene-1-oxide.

336. The composition of any one of claims 1-19, wherein the molecule is tetrahydrocannabinol-C₄, which has CAS Registry Number 60008-00-6 and is related to The Merck Index monograph M10624; and the anion is 3-butyl-6,6,9-trimethyl-6a,7,8,10a-tetrahydro-6H-benzo[c]chromene-1-oxide.

337. The composition of any one of claims 1-19, wherein the molecule is tetrahydrocannabiorcol, which has CAS Registry Number 22972-65-2 and is related to The Merck Index monograph M10624; and the anion is 3,6,6,9-tetramethyl-6a,7,8,10a-tetrahydro-6H-benzo[c]chromene-1-oxide.

338. The composition of any one of claims 1-19, wherein the molecule is tetrahydrocannabiphorol, which has CAS Registry Number 54763-99-4 and is related to The Merck Index monograph M10624; and the anion is 3-heptyl-6,6,9-trimethyl-6a,7,8,10a-tetrahydro-6H-benzo[c]chromene-1-oxide.

339. The composition of any one of claims 1-19, wherein the molecule is tetrahydrocannabivarin, which has CAS Registry Number 31262-37-0 and is related to The Merck Index monograph M10624; and the anion is 6,6,9-trimethyl-3-propyl-6a,7,8,10a-tetrahydro-6H-benzo[c]chromene-1-oxide.

340. The composition of any one of claims 1-19, wherein the molecule is tetrahydrorottlerin, which has CAS Registry Number 847152-13-0 and is described in The Merck Index monograph M9675; and the anion is selected from 7-hydroxy-2,2-dimethyl-8-(1-oxo-3-phenylpropyl)-6-[(3-acetyl-2,4,6-trihydroxy-5-methylphenyl)methyl]-3,4-dihydro-2H-chromene-5-oxide; 5-hydroxy-2,2-dimethyl-8-(1-oxo-3-phenylpropyl)-6-[(3-acetyl-2,4,6-trihydroxy-5-methylphenyl)methyl]-3,4-dihydro-2H-chromene-7-oxide; 2-acetyl-3,5-dihydroxy-4-methyl-6-{[5,7-dihydroxy-2,2-dimethyl-8-(1-oxo-3-phenylpropyl)-3,4-dihydro-2H-chromen-6-yl]methyl}phenolate; 4-acetyl-3,5-dihydroxy-6-methyl-2-{[5,7-dihydroxy-2,2-dimethyl-8-(1-oxo-3-phenylpropyl)-3,4-dihydro-2H-chromen-6-yl]methyl}phenolate; and 2-acetyl-3,5-dihydroxy-6-methyl-4-{[5,7-dihydroxy-2,2-dimethyl-8-(1-oxo-3-phenylpropyl)-3,4-dihydro-2H-chromen-6-yl]methyl}phenolate.

341. The composition of any one of claims 1-19, wherein the molecule is thymol, which has CAS Registry Number 89-83-8 and is described in The Merck Index monograph M10824; and the anion is 2-isopropyl-5-methylphenolate.

342. The composition of any one of claims 1-19, wherein the molecule is tioclomarol, which has CAS Registry Number 22619-35-8 and is described in The Merck Index monograph M10877; and the anion is 2-oxo-3-[3-hydroxy-3-(4-chlorophenyl)-1-(5-chlorothiophen-2-yl)propyl]-2H-chromene-4-oxide.

343. The composition of any one of claims 1-19, wherein the molecule is tolcapone, which has CAS Registry Number 134308-13-7 and is described in The Merck Index monograph M10938; and the anion is selected from 2-hydroxy-3-nitro-5-[(4-methylphenyl)carbonyl]phenolate and 6-hydroxy-2-nitro-4-[(4-methylphenyl)carbonyl]phenolate.

344. The composition of any one of claims 1-19, wherein the molecule is trilostane, which has CAS Registry Number 13647-35-3 and is described in The Merck Index monograph M11137; and the anion is 2-cyano-4,5-epoxy-17-hydroxy-10,13-dimethyl-1,4,6,7,8,9,11,12,14,15,16,17-dodecahydrocyclopenta[a]phenanthrene-3-oxide.

345. The composition of any one of claims 1-19, wherein the molecule is tyrosol, which has CAS Registry Number 501-94-0 and is described in The Merck Index monograph M11292; and the anion is 4-(2-hydroxyethyl)phenolate.

346. The composition of any one of claims 1-19, wherein the molecule is uliginosin A, which has CAS Registry Number 19809-78-0 and is described in The Merck Index monograph M11298; and the anion is selected from 5-hydroxy-6,6-dimethyl-3-oxo-4-(2-methyl-1-oxopropyl)-2-{[2,4,6-trihydroxy-5-isoprenyl-3-(2-methyl-1-oxopropyl)phenyl]methyl}cyclohexa-1,4-diene-1-oxide; 5-hydroxy-6,6-dimethyl-3-oxo-2-(2-methyl-1-oxopropyl)-4-{[2,4,6-trihydroxy-5-isoprenyl-3-(2-methyl-1-oxopropyl)phenyl]methyl}cyclohexa-1,4-diene-1-oxide; 3,5-dihydroxy-4-isoprenyl-6-(2-methyl-1-oxopropyl)-2-{[2,4-dihydroxy-3,3-dimethyl-6-oxo-5-(2-methyl-1-oxopropyl)cyclohexa-1,4-dien-1-yl]methyl}phenolate; 3,5-dihydroxy-6-isoprenyl-4-(2-methyl-1-oxopropyl)-2-{[2,4-dihydroxy-3,3-dimethyl-6-oxo-5-(2-methyl-1-oxopropyl)cyclohexa-1,4-dien-1-yl]methyl}phenolate; and 3,5-dihydroxy-6-isoprenyl-2-(2-methyl-1-oxopropyl)-4-{[2,4-dihydroxy-3,3-dimethyl-6-oxo-5-(2-methyl-1-oxopropyl)cyclohexa-1,4-dien-1-yl]methyl}phenolate.

347. The composition of any one of claims 1-19, wherein the molecule is uliginosin B, which has CAS Registry Number 19809-79-1 and is described in The Merck Index monograph M11298; and the anion is selected from 5-hydroxy-6,6-dimethyl-3-oxo-4-(2-methyl-1-oxopropyl)-2-{[5,7-dihydroxy-2,2-dimethyl-8-(2-methyl-1-oxopropyl)-2H-chromen-6-yl]methyl}cyclohexa-1,4-dien-1-oxide; 5-hydroxy-6,6-dimethyl-3-oxo-2-(2-methyl-1-oxopropyl)-4-{[5,7-dihydroxy-2,2-dimethyl-8-(2-methyl-1-oxopropyl)-2H-chromen-6-yl]methyl}cyclohexa-1,4-dien-1-oxide; 7-hydroxy-2,2-dimethyl-8-(2-methyl-1-oxopropyl)-6-{[2,4-dihydroxy-3,3-dimethyl-6-oxo-5-(2-methyl-1-oxopropyl)cyclohexa-1,4-dien-1-yl]methyl}-2H-chromene-5-oxide; and 5-hydroxy-2,2-dimethyl-8-(2-methyl-1-oxopropyl)-6-{[2,4-dihydroxy-3,3-dimethyl-6-oxo-5-(2-methyl-1-oxopropyl)cyclohexa-1,4-dien-1-yl]methyl}-2H-chromene-7-oxide.

348. The composition of any one of claims 1-19, wherein the molecule is umbelliferone, which has CAS Registry Number 93-35-6 and is described in The Merck Index monograph M11302; and the anion is 2-oxo-2H-chromene-7-oxide.

349. The composition of any one of claims 1-19, wherein the molecule is vanillin, which has CAS Registry Number 121-33-5 and is described in The Merck Index monograph M11390; and the anion is 4-formyl-2-methoxyphenolate.

350. The composition of any one of claims 1-19, wherein the molecule is vanillyl alcohol, which has CAS Registry Number 498-00-0 and is related to The Merck Index monograph M11390; and the anion is 4-(hydroxymethyl)-2-methoxyphenolate.

351. The composition of any one of claims 1-19, wherein the molecule is violacein, which has CAS Registry Number 548-54-9 and is described in The Merck Index monograph M11463; and the anion is 3-[5-oxo-4-(2-oxo-1H-indol-3-ylidene)-1H-pyrrol-2-yl]-1H-indol-5-oxide.

352. The composition of any one of claims 1-19, wherein the molecule is viridicatin, which has CAS Registry Number 129-24-8 and is described in The Merck Index monograph M11471; and the anion is 4-phenyl-2-oxo-1H-quinolin-3-oxide.

353. The composition of any one of claims 1-19, wherein the molecule is xanthoxylin, which has CAS Registry Number 90-24-4 and is described in The Merck Index monograph M11535; and the anion is 2-acetyl-3,5-dimethoxyphenolate.

354. The composition of any one of claims 1-19, wherein the molecule is xibornol, which has CAS Registry Number 13741-18-9 and is described in The Merck Index monograph M11543; and the anion is 4,5-dimethyl-2-[1,7,7-trimethylbicyclo[2.2.1]hept-2-yl]phenolate.

355. The composition of any one of claims 1-19, wherein the molecule is zearalenone, which has CAS Registry Number 17924-92-4 and is described in The Merck Index monograph M11584; and the anion is selected from 18-hydroxy-4-methyl-2,8-dioxo-3-oxabicyclo[12.4.0]octadeca-12,14,16,18-tetraene-16-oxide and 16-hydroxy-4-methyl-2,8-dioxo-3-oxabicyclo[12.4.0]octadeca-12,14,16,18-tetraene-18-oxide.

356. The composition of any one of claims 1-19, wherein the molecule is zeranol, which has CAS Registry Number 26538-44-3 and is described in The Merck Index monograph M11589; and the anion is selected from 8,18-dihydroxy-4-methyl-2-oxo-3-oxabicyclo[12.4.0]octadeca-14,16,18-triene-16-oxide and 8,16-dihydroxy-4-methyl-2-oxo-3-oxabicyclo[12.4.0]octadeca-14,16,18-triene-18-oxide.

357. The composition of any one of claims 1-19, wherein the molecule is zeta2-tocopherol, which has CAS Registry Number 17976-95-3 and is described in The Merck Index monograph M500; and the anion is 2,5,7-trimethyl-2-(4,8,12-trimethyltridecyl)-3,4-dihydro-2H-chromene-6-oxide.

358. The composition of any one of claims 1-19, wherein the molecule is zingerone, which has CAS Registry Number 122-48-5 and is described in The Merck Index monograph M11636; and the anion is 4-(3-oxobutyl)-2-methoxyphenolate.

359. The composition of any one of claims 1-19, wherein the molecule is [1]-gingerol, which has CAS Registry Number 99742-03-7 and is related to The Merck Index monograph M5729; and the anion is 2-methoxy-4-(5-hydroxy-3-oxopentyl)phenolate.

360. The composition of any one of claims 1-19, wherein the molecule is [10]-gingerol, which has CAS Registry Number 23513-15-7 and is related to The Merck Index monograph M5729; and the anion is 2-methoxy-4-(5-hydroxy-3-oxotetradecyl)phenolate.

361. The composition of any one of claims 1-19, wherein the molecule is [12]-gingerol, which has CAS Registry Number 104264-55-3 and is related to The Merck Index monograph M5729; and the anion is 2-methoxy-4-(5-hydroxy-3-oxohexadecyl)phenolate.

362. The composition of any one of claims 1-19, wherein the molecule is [14]-gingerol, which is related to The Merck Index monograph M5729; and the anion is 2-methoxy-4-(5-hydroxy-3-oxooctadecyl)phenolate.

363. The composition of any one of claims 1-19, wherein the molecule is [16]-gingerol, which is related to The Merck Index monograph M5729; and the anion is 2-methoxy-4-(5-hydroxy-3-oxoicosyl)phenolate.

364. The composition of any one of claims 1-19, wherein the molecule is [2]-gingerol, which is related to The Merck Index monograph M5729; and the anion is 2-methoxy-4-(5-hydroxy-3-oxohexyl)phenolate.

365. The composition of any one of claims 1-19, wherein the molecule is [3]-gingerol, which has CAS Registry Number 41743-67-3 and is related to The Merck Index monograph M5729; and the anion is 2-methoxy-4-(5-hydroxy-3-oxoheptyl)phenolate.

366. The composition of any one of claims 1-19, wherein the molecule is [4]-gingerol, which has CAS Registry Number 77398-90-4 and is related to The Merck Index monograph M5729; and the anion is 2-methoxy-4-(5-hydroxy-3-oxooctyl)phenolate.

367. The composition of any one of claims 1-19, wherein the molecule is [5]-gingerol, which has CAS Registry Number 41743-69-5 and is related to The Merck Index monograph M5729; and the anion is 2-methoxy-4-(5-hydroxy-3-oxononyl)phenolate.

368. The composition of any one of claims 1-19, wherein the molecule is [6]-gingerol, which has CAS Registry Number 23513-14-6 and is described in The Merck Index monograph M5729; and the anion is 2-methoxy-4-(5-hydroxy-3-oxodecyl)phenolate.

369. The composition of any one of claims 1-19, wherein the molecule is [7]-gingerol, which is related to The Merck Index monograph M5729; and the anion is 2-methoxy-4-(5-hydroxy-3-oxoundecyl)phenolate.

370. The composition of any one of claims 1-19, wherein the molecule is [8]-gingerol, which has CAS Registry Number 23513-08-8 and is related to The Merck Index monograph M5729; and the anion is 2-methoxy-4-(5-hydroxy-3-oxododecyl)phenolate.

371. The composition of any one of claims 1-19, wherein the molecule is [9]-gingerol, which is related to The Merck Index monograph M5729; and the anion is 2-methoxy-4-(5-hydroxy-3-oxotridecyl)phenolate.

372. The composition of any one of claims 1-19, wherein the molecule is 16-epiestriol, which has CAS Registry Number 547-81-9 and is described in The Merck Index monograph M4941; and the anion is 16,17-dihydroxy-13-methyl-6,7,8,9,11,12,14,15,16,17-decahydrocyclopenta[a]phenanthrene-3-oxide.

373. The composition of any one of claims 1-19, wherein the molecule is 17alpha-dihydroequilin, which has CAS Registry Number 651-55-8 and is described in The Merck Index monograph M4460; and the anion is 17-hydroxy-13-methyl-6,9,11,12,14,15,16,17-octahydrocyclopenta[a]phenanthrene-3-oxide.

374. The composition of any one of claims 1-19, wherein the molecule is 2,5-di-tert-pentylhydroquinone, which has CAS Registry Number 79-74-3 and is described in The Merck Index monograph M4604; and the anion is 4-hydroxy-2,5-bis(2-methylbut-2-yl)phenolate.

375. The composition of any one of claims 1-19, wherein the molecule is 2′,7′-dichlorofluorescein, which has CAS Registry Number 76-54-0 and is described in The Merck Index monograph M4346; and the anion is 2′,7′-dichloro-6′-hydroxy-3-oxospiro(2-benzofuran-1,9′-xanthene)-3′-oxide.

376. The composition of any one of claims 1-19, wherein the molecule is 2-naphthol, which has CAS Registry Number 135-19-3 and is described in The Merck Index monograph M7739; and the anion is naphthalene-2-oxide.

377. The composition of any one of claims 1-19, wherein the molecule is 3,6-dimethylmangostin, which has CAS Registry Number 15404-76-9 and is described in The Merck Index monograph M7077; and the anion is 2,8-diisoprenyl-3,6,7-trimethoxy-9-oxoxanthene-1-oxide.

378. The composition of any one of claims 1-19, wherein the molecule is 3-methoxy-4-hydroxyphenylglycol, which has CAS Registry Number 534-82-7 and is described in The Merck Index monograph M7339; and the anion is 2-methoxy-4-(1,2-dihydroxyethyl)phenolate.

379. The composition of any one of claims 1-19, wherein the molecule is 4′,5′-dichlorofluorescein, which has CAS Registry Number 2320-96-9 and is described in The Merck Index monograph M4347; and the anion is 4′,5′-dichloro-6′-hydroxy-3-oxospiro(2-benzofuran-1,9′-xanthene)-3′-oxide.

380. The composition of any one of claims 1-19, wherein the molecule is 4′,5′-diiodofluorescein, which has CAS Registry Number 38577-97-8 and is described in The Merck Index monograph M4477; and the anion is 6′-hydroxy-4′,5′-diiodo-3-oxospiro(2-benzofuran-1,9′-xanthene)-3′-oxide.

381. The composition of any one of claims 1-19, wherein the molecule is 4′-fluorocannabidiol, which has CAS Registry Number 1619228-89-5 and is related to The Merck Index monograph M3020; and the anion is selected from 4-fluoro-3-hydroxy-5-pentyl-2-(3-methyl-6-isopropenylcyclohex-2-enyl)phenolate and 6-fluoro-3-hydroxy-5-pentyl-2-(3-methyl-6-isopropenylcyclohex-2-enyl)phenolate.

382. The composition of any one of claims 1-19, wherein the molecule is 4-hexylresorcinol, which has CAS Registry Number 136-77-6 and is described in The Merck Index monograph M6018; and the anion is selected from 4-hexyl-3-hydroxyphenolate and 6-hexyl-3-hydroxyphenolate.

383. The composition of any one of claims 1-19, wherein the molecule is 4-O-methylhonokiol, which has CAS Registry Number 68592-15-4 and is related to The Merck Index monograph M6051; and the anion is 4-(prop-2-enyl)-2-[4-methoxy-3-(prop-2-enyl)phenyl]phenolate.

384. The composition of any one of claims 1-19, wherein the molecule is 4′-O-methylkaempferol, which has CAS Registry Number 491-54-3 and is related to The Merck Index monograph M6592; and the anion is selected from 5,7-dihydroxy-4-oxo-2-(4-methoxyphenyl)-4H-chromene-3-oxide; 3,7-dihydroxy-4-oxo-2-(4-methoxyphenyl)-4H-chromene-5-oxide; and 3,5-dihydroxy-4-oxo-2-(4-methoxyphenyl)-4H-chromene-7-oxide.

385. The composition of any one of claims 1-19, wherein the molecule is 5-ethoxy-10-gingerol, which is related to The Merck Index monograph M5729; and the anion is 2-methoxy-4-(5-ethoxy-3-oxotetradecyl)phenolate.

386. The composition of any one of claims 1-19, wherein the molecule is 5-O-methyl-9-gingerol, which is related to The Merck Index monograph M5729; and the anion is 2-methoxy-4-(5-methoxy-3-oxotridecyl)phenolate.

387. The composition of any one of claims 1-19, wherein the molecule is 6-deoxy-gamma mangostin, which has CAS Registry Number 105037-94-3 and is related to The Merck Index monograph M7077; and the anion is selected from 3,7-dihydroxy-2,8-diisoprenyl-9-oxoxanthene-1-oxide; 6,8-dihydroxy-1,7-diisoprenyl-9-oxoxanthene-2-oxide; and 1,7-dihydroxy-2,8-diisoprenyl-9-oxoxanthene-3-oxide.

388. The composition of any one of claims 1-19, wherein the molecule is 8-isoestrone, which has CAS Registry Number 517-06-6 and is described in The Merck Index monograph M6483; and the anion is 13-methyl-17-oxo-7,8,9,11,12,14,15,16-octahydro-6H-cyclopenta[a]phenanthrene-3-oxide.

389. The composition of any one of claims 1-19, wherein the molecule is 9-hydroxy-[6]-gingerol, which is related to The Merck Index monograph M5729; and the anion is 2-methoxy-4-(5,9-dihydroxy-3-oxodecyl)phenolate.

390. The composition of any one of claims 1-19, wherein the molecule is abediterol, which has CAS Registry Number 915133-65-2; and the anion is 2-oxo-5-[1-hydroxy-2-(N-{6-[(2,2-difluoro-2-phenylethyl)oxy]hexyl}amino)ethyl]-1H-quinoline-8-oxide.

391. The composition of any one of claims 1-19, wherein the molecule is abnormal cannabidiol, which has CAS Registry Number 22972-55-0; and the anion is 3-hydroxy-5-pentyl-4-(3-methyl-6-isopropenylcyclohex-2-enyl)phenolate.

392. The composition of any one of claims 1-19, wherein the molecule is acerosin, which has CAS Registry Number 15835-74-2; and the anion is selected from 7-hydroxy-6,8-dimethoxy-4-oxo-2-(3-hydroxy-4-methoxyphenyl)-4H-chromene-5-oxide; 5-hydroxy-6,8-dimethoxy-4-oxo-2-(3-hydroxy-4-methoxyphenyl)-4H-chromene-7-oxide; and 2-methoxy-5-(5,7-dihydroxy-6,8-dimethoxy-4-oxo-4H-chromen-2-yl)phenolate.

393. The composition of any one of claims 1-19, wherein the molecule is acetosyringone, which has CAS Registry Number 2478-38-8; and the anion is 4-acetyl-2,6-dimethoxyphenolate.

394. The composition of any one of claims 1-19, wherein the molecule is adipostatin A, which has CAS Registry Number 3158-56-3; and the anion is 3-hydroxy-5-pentadecylphenolate.

395. The composition of any one of claims 1-19, wherein the molecule is afzelechin, which has CAS Registry Number 2545-00-8; and the anion is selected from 3,7-dihydroxy-2-(4-hydroxyphenyl)-3,4-dihydro-2H-chromene-5-oxide; 3,5-dihydroxy-2-(4-hydroxyphenyl)-3,4-dihydro-2H-chromene-7-oxide; and 4-(3,5,7-trihydroxy-3,4-dihydro-2H-chromen-2-yl)phenolate.

396. The composition of any one of claims 1-19, wherein the molecule is aleuritin, which has CAS Registry Number 124901-94-6; and the anion is 2,6-dimethoxy-4-[2-hydroxymethyl-5-methoxy-8-oxo-2,3-dihydropyrano[2,3-h][1,4]benzodioxin-3-yl]phenolate.

397. The composition of any one of claims 1-19, wherein the molecule is alnetin, which has CAS Registry Number 3151-82-4; and the anion is 6,7,8-trimethoxy-2-phenyl-4-oxo-4H-chromene-5-oxide.

398. The composition of any one of claims 1-19, wherein the molecule is alternariol, which has CAS Registry Number 641-38-3; and the anion is 7,9-dihydroxy-1-methyl-6-oxo-6H-benzo[c]chromene-3-oxide.

399. The composition of any one of claims 1-19, wherein the molecule is AM-087, which has CAS Registry Number 152674-96-9; and the anion is 3-(6-bromo-2-methylhex-2-yl)-6,6,9-trimethyl-6a,7,10,10a-tetrahydrobenzo[c]chromene-1-oxide.

400. The composition of any one of claims 1-19, wherein the molecule is AM-1714, which has CAS Registry Number 335371-37-4; and the anion is 9-hydroxy-3-(2-methyloct-2-yl)-6-oxo-6H-benzo[c]chromene-1-oxide.

401. The composition of any one of claims 1-19, wherein the molecule is AM-2389, which has CAS Registry Number 1256842-49-5; and the anion is 3-(1-hexylcyclobutyl)-9-hydroxy-6,6-dimethyl-6a,7,8,9,10,10a-hexahydro-6H-benzo[c]chromene-1-oxide.

402. The composition of any one of claims 1-19, wherein the molecule is AM-4030, which has CAS Registry Number 587023-54-9; and the anion is 9-(hydroxymethyl)-6-(3-hydroxyprop-1-enyl)-6-methyl-3-(2-methyloct-2-yl)-6a,7,8,9,10,10a-hexahydro-6H-benzo[c]chromene-1-oxide.

403. The composition of any one of claims 1-19, wherein the molecule is AM404, which has CAS Registry Number 183718-77-6; and the anion is 4-[N-(1-oxoicosa-5,8,11,14-tetraenyl)amino]phenolate.

404. The composition of any one of claims 1-19, wherein the molecule is AM-411, which has CAS Registry Number 212835-02-4; and the anion is 3-(1-adamantyl)-6,6,9-trimethyl-6a,7,10,10a-tetrahydro-6H-benzo[c]chromene-1-oxide.

405. The composition of any one of claims 1-19, wherein the molecule is AM-855, which has CAS Registry Number 249888-50-4; and the anion is 8-hexyl-2,5,5-trimethyl-1,4,4a,8,9,10,11,12b-octahydronaphtho[3,2-c]isochromen-12-oxide.

406. The composition of any one of claims 1-19, wherein the molecule is AM-905, which has CAS Registry Number 181139-62-8; and the anion is 3-(hept-1-enyl)-9-(hydroxymethyl)-6,6-dimethyl-6a,7,8,9,10,10a-hexahydro-6H-benzo[c]chromene-1-oxide.

407. The composition of any one of claims 1-19, wherein the molecule is AM-919, which has CAS Registry Number 164228-46-0; and the anion is 9-(hydroxymethyl)-6-(3-hydroxypropyl)-6-methyl-3-(2-methyloct-2-yl)-6a,7,8,9,10,10a-hexahydro-6H-benzo[c]chromene-1-oxide.

408. The composition of any one of claims 1-19, wherein the molecule is AM-938, which has CAS Registry Number 303113-08-8; and the anion is 9-(hydroxymethyl)-6-(3-hydroxyprop-1-ynyl)-6-methyl-3-(2-methyloct-2-yl)-6a,7,8,9,10,10a-hexahydro-6H-benzo[c]chromene-1-oxide.

409. The composition of any one of claims 1-19, wherein the molecule is amentoflavone, which has CAS Registry Number 1617-53-4; and the anion is selected from 7-hydroxy-2-(4-hydroxyphenyl)-4-oxo-8-[2-hydroxy-5-(5,7-dihydroxy-4-oxo-4H-chromen-2-yl)phenyl]-4H-chromene-5-oxide; 5-hydroxy-2-(4-hydroxyphenyl)-4-oxo-8-[2-hydroxy-5-(5,7-dihydroxy-4-oxo-4H-chromen-2-yl)phenyl]-4H-chromene-7-oxide; 4-{5,7-dihydroxy-4-oxo-8-[2-hydroxy-5-(5,7-dihydroxy-4-oxo-4H-chromen-2-yl)phenyl]-4H-chromen-2-yl}phenolate; 7-hydroxy-4-oxo-2-[4-hydroxy-5-(5,7-dihydroxy-2-(4-hydroxyphenyl)-4-oxo-4H-chromen-8-yl)phenyl]-4H-chromene-5-oxide; and 5-hydroxy-4-oxo-2-[4-hydroxy-5-(5,7-dihydroxy-2-(4-hydroxyphenyl)-4-oxo-4H-chromen-8-yl)phenyl]-4H-chromene-7-oxide.

410. The composition of any one of claims 1-19, wherein the molecule is AMG-1, which has CAS Registry Number 205746-46-9; and the anion is 3-(hept-1-ynyl)-6,6,9-trimethyl-6a,7,10,10a-tetrahydro-6H-benzo[c]chromene-1-oxide.

411. The composition of any one of claims 1-19, wherein the molecule is AMG-36, which has CAS Registry Number 579444-43-2; and the anion is 3-(1-hexylcyclopentyl)-6,6,9-trimethyl-6a,7,10,10a-tetrahydro-6H-benzo[c]chromene-1-oxide.

412. The composition of any one of claims 1-19, wherein the molecule is AMG-41, which has CAS Registry Number 499237-35-3; and the anion is 3-(1-hexylcyclopropyl)-6,6,9-trimethyl-6a,7,10,10a-tetrahydro-6H-benzo[c]chromene-1-oxide.

413. The composition of any one of claims 1-19, wherein the molecule is amylmetacresol, which has CAS Registry Number 1300-94-3; and the anion is 5-methyl-2-pentylphenolate.

414. The composition of any one of claims 1-19, wherein the molecule is apiforol, which has CAS Registry Number 55167-29-8; and the anion is selected from 4,7-dihydroxy-2-(4-hydroxyphenyl)-3,4-dihydro-2H-chromene-5-oxide; 4,5-dihydroxy-2-(4-hydroxyphenyl)-3,4-dihydro-2H-chromene-7-oxide; and 4-(4,5,7-trihydroxy-3,4-dihydro-2H-chromen-2-yl)phenolate.

415. The composition of any one of claims 1-19, wherein the molecule is arctigenin, which has CAS Registry Number 7770-78-7; and the anion is 2-methoxy-4-({2-oxo-5-[(3,4-dimethoxyphenyl)methyl]-3-oxacyclopentyl}methyl)phenolate.

416. The composition of any one of claims 1-19, wherein the molecule is artocarpetin; and the anion is selected from 7-methoxy-4-oxo-2-(2,4-dihydroxyphenol)-4H-chromene-5-oxide; 3-hydroxy-4-(5-hydroxy-7-methoxy-4-oxo-4H-chromen-2-yl)phenolate; and 3-hydroxy-6-(5-hydroxy-7-methoxy-4-oxo-4H-chromen-2-yl)phenolate.

417. The composition of any one of claims 1-19, wherein the molecule is artocarpin, which has CAS Registry Number 7608-44-8; and the anion is selected from 3,6-diisoprenyl-7-methoxy-4-oxo-2-(2,4-dihydroxyphenyl)-4H-chromene-5-oxide; 3-hydroxy-4-(5-hydroxy-3,6-diisoprenyl-7-methoxy-4-oxo-4H-chromen-2-yl)phenolate; and 3-hydroxy-6-(5-hydroxy-3,6-diisoprenyl-7-methoxy-4-oxo-4H-chromen-2-yl)phenolate.

418. The composition of any one of claims 1-19, wherein the molecule is ascofuranone, which has CAS Registry Number 38462-04-3; and the anion is selected from 2-chloro-4-formyl-5-hydroxy-3-methyl-6-[3-methyl-7-(3-oxo-4,4-dimethyl-5-oxacyclopentyl)octa-2,6-dienyl]phenolate and 4-chloro-6-formyl-3-hydroxy-5-methyl-2-[3-methyl-7-(3-oxo-4,4-dimethyl-5-oxacyclopentyl)octa-2,6-dienyl]phenolate.

419. The composition of any one of claims 1-19, wherein the molecule is aureusidin, which has CAS Registry Number 38216-54-5; and the anion is selected from 4-hydroxy-3-oxo-2-[(3,4-dihydroxyphenyl)methylidene]-1-benzofuran-6-oxide; 6-hydroxy-3-oxo-2-[(3,4-dihydroxyphenyl)methylidene]-1-benzofuran-4-oxide; 2-hydroxy-4-[(4,6-dihydroxy-3-oxo-1-benzofuran-2-ylidene)methyl]phenolate; and 2-hydroxy-5-[(4,6-dihydroxy-3-oxo-1-benzofuran-2-ylidene)methyl]phenolate.

420. The composition of any one of claims 1-19, wherein the molecule is axillarin, which has CAS Registry Number 5188-73-8; and the anion is selected from 5-hydroxy-3,6-dimethoxy-4-oxo-2-(3,4-dihydroxyphenyl)-4H-chromene-7-oxide; 7-hydroxy-3,6-dimethoxy-4-oxo-2-(3,4-dihydroxyphenyl)-4H-chromene-5-oxide; 2-hydroxy-4-(5,7-dihydroxy-3,6-dimethoxy-4-oxo-4H-chromen-2-yl)phenolate; and 2-hydroxy-5-(5,7-dihydroxy-3,6-dimethoxy-4-oxo-4H-chromen-2-yl)phenolate.

421. The composition of any one of claims 1-19, wherein the molecule is azaleatin, which has CAS Registry Number 529-51-1; and the anion is selected from 7-hydroxy-5-methoxy-4-oxo-2-(2,4-dihydroxyphenyl)-4H-chromene-3-oxide; 3-hydroxy-5-methoxy-4-oxo-2-(2,4-dihydroxyphenyl)-4H-chromene-7-oxide; 2-hydroxy-4-(3,7-dihydroxy-5-methoxy-4-oxo-4H-chromen-2-yl)phenolate; and 2-hydroxy-5-(3,7-dihydroxy-5-methoxy-4-oxo-4H-chromen-2-yl)phenolate.

422. The composition of any one of claims 1-19, wherein the molecule is bakuchiol, which has CAS Registry Number 10309-37-2; and the anion is 4-(3-ethenyl-3,7-dimethylocta-1,6-dienyl)phenolate.

423. The composition of any one of claims 1-19, wherein the molecule is balanophonin, which has CAS Registry Number 118916-57-7; and the anion is 2-methoxy-4-[3-hydroxymethyl-7-methoxy-5-(3-oxoprop-1-enyl)-2,3-dihydro-1-benzofuran-2-yl]phenolate.

424. The composition of any one of claims 1-19, wherein the molecule is benzophenone-2, which has CAS Registry Number 131-55-5; and the anion is selected from 3-hydroxy-4-[(2,4-dihydroyphenyl)carbonyl]phenolate and 3-hydroxy-6-[(2,4-dihydroyphenyl)carbonyl]phenolate.

425. The composition of any one of claims 1-19, wherein the molecule is bilobol, which has CAS Registry Number 22910-86-7; and the anion is 3-hydroxy-5-(pentadec-8-enyl)phenolate.

426. The composition of any one of claims 1-19, wherein the molecule is bisdemethoxycurcumin, which has CAS Registry Number 24939-16-0; and the anion is 4-[3,5-dioxo-7-(4-hydroxyphenyl)hepta-1,6-dieneyl]phenolate.

427. The composition of any one of claims 1-19, wherein the molecule is bisphenol F, which has CAS Registry Number 620-92-8; and the anion is 4-[(4-hydroxyphenyl)methyl]phenolate.

428. The composition of any one of claims 1-19, wherein the molecule is blumeatin, which has CAS Registry Number 118024-26-3; and the anion is selected from 7-methoxy-4-oxo-2-(3,5-dihydroxyphenyl)-2,3-dihydro-4H-chromene-5-oxide and 3-hydroxy-5-(5-hydroxy-7-methoxy-4-oxo-2,3-dihydro-4H-chromen-2-yl)phenolate.

429. The composition of any one of claims 1-19, wherein the molecule is butin, which has CAS Registry Number 492-14-8; and the anion is selected from 4-oxo-2-(3,4-dihydroxyphenyl)-2,3-dihydro-4H-chromene-7-oxide; 2-hydroxy-4-(7-hydroxy-4-oxo-2,3-dihydro-4H-chromen-2-yl)phenolate; and 2-hydroxy-5-(7-hydroxy-4-oxo-2,3-dihydro-4H-chromen-2-yl)phenolate.

430. The composition of any one of claims 1-19, wherein the molecule is caflanone, which has CAS Registry Number 199167-23-2; and the anion is selected from 7-hydroxy-8-isoprenyl-4-oxo-2-(4-hydroxy-3-methoxyphenyl)-4H-chromene-5-oxide; 5-hydroxy-8-isoprenyl-4-oxo-2-(4-hydroxy-3-methoxyphenyl)-4H-chromene-7-oxide; and 2-methoxy-4-(5,7-dihydroxy-8-isoprenyl-4-oxo-4H-chromen-2-yl)phenolate.

431. The composition of any one of claims 1-19, wherein the molecule is calphostin A, which has CAS Registry Number 120461-92-9; and the anion is 10-hydroxy-2,6,7,11-tetramethoxy-4,9-dioxo-1,12-bis[2-(benzoyloxy)propyl]perylene-3-oxide.

432. The composition of any one of claims 1-19, wherein the molecule is calphostin B, which has CAS Registry Number 124824-06-2; and the anion is selected from 10-hydroxy-2,6,7,11-tetramethoxy-4,9-dioxo-12-(2-hydroxypropyl)-1-[2-(benzoyloxy)propyl]perylene-3-oxide and 10-hydroxy-2,6,7,11-tetramethoxy-4,9-dioxo-1-(2-hydroxypropyl)-12-[2-(benzoyloxy)propyl]perylene-3-oxide.

433. The composition of any one of claims 1-19, wherein the molecule is calphostin C, which has CAS Registry Number 121263-19-2; and the anion is selected from 10-hydroxy-2,6,7,11-tetramethoxy-4,9-dioxo-12-[2-(benzoyloxy)propyl]-1-[2-({[(4-hydroxyphenyl)oxy]carbonyl}oxy)propyl]perylene-3-oxide and 10-hydroxy-2,6,7,11-tetramethoxy-4,9-dioxo-1-[2-(benzoyloxy)propyl]-12-[2-({[(4-hydroxyphenyl)oxy]carbonyl}oxy)propyl]perylene-3-oxide.

434. The composition of any one of claims 1-19, wherein the molecule is calphostin D, which has CAS Registry Number 124986-26-1; and the anion is 10-hydroxy-2,6,7,11-tetramethoxy-4,9-dioxo-1,12-bis(2-hydroxypropyl)perylene-3-oxide.

435. The composition of any one of claims 1-19, wherein the molecule is calycosin, which has CAS Registry Number 20575-57-9; and the anion is selected from 4-oxo-3-(3-hydroxy-4-methoxyphenyl)-4H-chromene-7-oxide and 2-methoxy-5-(7-hydroxy-4-oxo-4H-chromen-3-yl)phenolate.

436. The composition of any one of claims 1-19, wherein the molecule is campesteryl ferulate, which has CAS Registry Number 20972-07-0; and the anion is 2-methoxy-4-(3-oxo-3-{[10,13-dimethyl-17-(5,6-dimethylhept-2-yl)-2,3,4,7,8,9,11,12,14,15,16,17-dodecahydro-1H-cyclopenta[a]phenanthren-3-yl]oxy}prop-1-enyl)phenolate.

437. The composition of any one of claims 1-19, wherein the molecule is canbisol, which has CAS Registry Number 56689-43-1; and the anion is 9-hydroxy-6,6-dimethyl-3-(2-methyloct-2-yl)-6a,7,8,9,10,10a-hexahydro-6H-benzo[c]chromene-1-oxide.

438. The composition of any one of claims 1-19, wherein the molecule is cannabichromene, which has CAS Registry Number 20675-51-8; and the anion is 2-methyl-7-pentyl-2-(4-methylpent-3-enyl)-2H-chromene-5-oxide.

439. The composition of any one of claims 1-19, wherein the molecule is cannabicyclohexanol, which has CAS Registry Number 70434-92-3; and the anion is 2-(3-hydroxycyclohexyl)-5-(2-methylnonan-2-yl)phenolate.

440. The composition of any one of claims 1-19, wherein the molecule is cannabicyclol, which has CAS Registry Number 21366-63-2; and the anion is 9,13,13-trimethyl-5-pentyl-8-oxatetracyclo[7.4.1.0^{2,7}.0^{12,14}]tetradeca-2,4,6-trien-3-oxide.

441. The composition of any one of claims 1-19, wherein the molecule is cannabicyclovarin; and the anion is 9,13,13-trimethyl-5-propyl-8-oxatetracyclo[7.4.1.0^{2,7}.0^{12,14}]tetradeca-2,4,6-trien-3-oxide.

442. The composition of any one of claims 1-19, wherein the molecule is cannabigerol, which has CAS Registry Number 25654-31-3; and the anion is 3-hydroxy-2-geranyl-5-pentylphenolate.

443. The composition of any one of claims 1-19, wherein the molecule is cannabigerovarin, which has CAS Registry Number 55824-11-8; and the anion is 3-hydroxy-2-geranyl-5-propylphenolate.

444. The composition of any one of claims 1-19, wherein the molecule is cannflavin A, which has CAS Registry Number 76735-57-4; and the anion is selected from 6-geranyl-7-hydroxy-4-oxo-2-(4-hydroxy-3-methoxyphenolate)-4H-chromene-5-oxide; 6-geranyl-5-hydroxy-4-oxo-2-(4-hydroxy-3-methoxyphenolate)-4H-chromene-7-oxide; and 2-methoxy-4-(6-geranyl-5,7-dihydroxy-4-oxo-4H-chromen-2-yl)phenolate.

445. The composition of any one of claims 1-19, wherein the molecule is cannflavin B, which has CAS Registry Number 76735-58-5; and the anion is selected from 7-hydroxy-6-isoprenyl-4-oxo-2-(4-hydroxy-3-methoxyphenolate)-4H-chromene-5-oxide; 5-hydroxy-6-isoprenyl-4-oxo-2-(4-hydroxy-3-methoxyphenolate)-4H-chromene-7-oxide; and 2-methoxy-4-(5,7-dihydroxy-6-isoprenyl-4-oxo-4H-chromen-2-yl)phenolate.

446. The composition of any one of claims 1-19, wherein the molecule is cannflavin C; and the anion is selected from 8-geranyl-7-hydroxy-4-oxo-2-(4-hydroxy-3-methoxyphenolate)-4H-chromene-5-oxide; 8-geranyl-5-hydroxy-4-oxo-2-(4-hydroxy-3-methoxyphenolate)-4H-chromene-7-oxide; and 2-methoxy-4-(8-geranyl-5,7-dihydroxy-4-oxo-4H-chromen-2-yl)phenolate.

447. The composition of any one of claims 1-19, wherein the molecule is canolol, which has CAS Registry Number 28343-22-8; and the anion is 4-ethenyl-2,6-dimethoxyphenolate.

448. The composition of any one of claims 1-19, wherein the molecule is chaetochromin A, which has CAS Registry Number 75514-37-3; and the anion is selected from 6,8-dihydroxy-2,3-dimethyl-4-oxo-9-(5,6,8-trihydroxy-2,3-dimethyl-4-oxo-2,3-dihydrobenzo[g]chromen-9-yl)-2,3-dihydrobenzo[g]chromene-5-oxide; 5,8-dihydroxy-2,3-dimethyl-4-oxo-9-(5,6,8-trihydroxy-2,3-dimethyl-4-oxo-2,3-dihydrobenzo[g]chromen-9-yl)-2,3-dihydrobenzo[g]chromene-6-oxide; and 5,6-dihydroxy-2,3-dimethyl-4-oxo-9-(5,6,8-trihydroxy-2,3-dimethyl-4-oxo-2,3-dihydrobenzo[g]chromen-9-yl)-2,3-dihydrobenzo[g]chromene-8-oxide.

449. The composition of any one of claims 1-19, wherein the molecule is chaetochromin C; and the anion is selected from 6,8-dihydroxy-2,3-dimethyl-4-oxo-9-(5,6,8-trihydroxy-2-methyl-4-oxo-2,3-dihydrobenzo[g]chromen-9-yl)-2,3-dihydrobenzo[g]chromene-5-oxide; 5,8-dihydroxy-2,3-dimethyl-4-oxo-9-(5,6,8-trihydroxy-2-methyl-4-oxo-2,3-dihydrobenzo[g]chromen-9-yl)-2,3-dihydrobenzo[g]chromene-6-oxide; 5,6-dihydroxy-2,3-dimethyl-4-oxo-9-(5,6,8-trihydroxy-2-methyl-4-oxo-2,3-dihydrobenzo[g]chromen-9-yl)-2,3-dihydrobenzo[g]chromene-8-oxide; 6,8-dihydroxy-2-methyl-4-oxo-9-(5,6,8-trihydroxy-2,3-dimethyl-4-oxo-2,3-dihydrobenzo[g]chromen-9-yl)-2,3-dihydrobenzo[g]chromene-5-oxide; 5,8-dihydroxy-2-methyl-4-oxo-9-(5,6,8-trihydroxy-2,3-dimethyl-4-oxo-2,3-dihydrobenzo[g]chromen-9-yl)-2,3-dihydrobenzo[g]chromene-6-oxide; and 5,6-dihydroxy-2-methyl-4-oxo-9-(5,6,8-trihydroxy-2,3-dimethyl-4-oxo-2,3-dihydrobenzo[g]chromen-9-yl)-2,3-dihydrobenzo[g]chromene-8-oxide.

450. The composition of any one of claims 1-19, wherein the molecule is chaetochromin D; and the anion is selected from 5,6-dihydroxy-2,3-dimethyl-8-oxo-9-(5,6,8-trihydroxy-2,3-dimethyl-4-oxo-2,3-dihydrobenzo[g]chromen-9-yl)benzo[g]chromene-4-oxide; 4,6-dihydroxy-2,3-dimethyl-8-oxo-9-(5,6,8-trihydroxy-2,3-dimethyl-4-oxo-2,3-dihydrobenzo[g]chromen-9-yl)benzo[g]chromene-5-oxide; 4,5-dihydroxy-2,3-dimethyl-8-oxo-9-(5,6,8-trihydroxy-2,3-dimethyl-4-oxo-2,3-dihydrobenzo[g]chromen-9-yl)benzo[g]chromene-6-oxide; 6,8-dihydroxy-2,3-dimethyl-4-oxo-9-(4,5,6-trihydroxy-2,3-dimethyl-8-oxo-benzo[g]chromen-9-yl)-2,3-dihydrobenzo[g]chromene-5-oxide; 5,8-dihydroxy-2,3-dimethyl-4-oxo-9-(4,5,6-trihydroxy-2,3-dimethyl-8-oxo-benzo[g]chromen-9-yl)-2,3-dihydrobenzo[g]chromene-6-oxide; and 5,6-dihydroxy-2,3-dimethyl-4-oxo-9-(4,5,6-trihydroxy-2,3-dimethyl-8-oxo-benzo[g]chromen-9-yl)-2,3-dihydrobenzo[g]chromene-8-oxide.

451. The composition of any one of claims 1-19, wherein the molecule is chavibetol, which has CAS Registry Number 501-19-9; and the anion is 2-methoxy-5-(prop-2-enyl)phenolate.

452. The composition of any one of claims 1-19, wherein the molecule is chrysoeriol, which has CAS Registry Number 491-71-4; and the anion is selected from 7-hydroxy-4-oxo-2-(4-hydroxy-3-methoxyphenyl)-4H-chromene-5-oxide; 5-hydroxy-4-oxo-2-(4-hydroxy-3-methoxyphenyl)-4H-chromene-7-oxide; and 2-methoxy-4-(5,7-dihydroxy-4-oxo-4H-chromen-2-yl)phenolate.

453. The composition of any one of claims 1-19, wherein the molecule is cirsilineol, which has CAS Registry Number 41365-32-6; and the anion is selected from 6,7-dimethoxy-4-oxo-2-(4-hydroxy-3-methoxyphenyl)-4H-chromene-5-oxide and 2-methoxy-4-(5-hydroxy-6,7-dimethoxy-4-oxo-4H-chromen-2-yl)phenolate.

454. The composition of any one of claims 1-19, wherein the molecule is cirsiliol, which has CAS Registry Number 34334-69-5; and the anion is selected from 6,7-dimethoxy-4-oxo-2-(3,4-dihydroxyphenyl)-4H-chromene-5-oxide; 2-hydroxy-4-(5-hydroxy-6,7-dimethoxy-4-oxo-4H-chromen-2-yl)phenolate; and 2-hydroxy-5-(5-hydroxy-6,7-dimethoxy-4-oxo-4H-chromen-2-yl)phenolate.

455. The composition of any one of claims 1-19, wherein the molecule is cirsimaritin, which has CAS Registry Number 6601-62-3; and the anion is selected from 6,7-dimethoxy-4-oxo-2-(4-hydroxyphenyl)-4H-chromene-5-oxide and 4-(5-hydroxy-6,7-dimethoxy-4-oxo-4H-chromen-2-yl)phenolate.

456. The composition of any one of claims 1-19, wherein the molecule is corymbosin, which has CAS Registry Number 18103-41-8; and the anion is 7-methoxy-4-oxo-2-(3,4,5-trimethoxyphenyl)-4H-chromene-5-oxide.

457. The composition of any one of claims 1-19, wherein the molecule is coumatetralyl, which has CAS Registry Number 5836-29-3; and the anion is 2-oxo-3-(1,2,3,4-tetrahydronaphthalen-1-yl)-2H-chromene-4-oxide.

458. The composition of any one of claims 1-19, wherein the molecule is CP 55,244, which has CAS Registry Number 79678-32-3; and the anion is 2-[3-hydroxy-7-(hydroxymethyl)-1,2,3,4,4a,5,6,7,8,8a-decahydronaphthalen-1-yl]-5-(2-methyloct-2-yl)phenolate.

459. The composition of any one of claims 1-19, wherein the molecule is CP 55,940, which has CAS Registry Number 83002-04-4; and the anion is 2-[3-hydroxy-6-(3-hydroxypropyl)cyclohexyl]-5-(2-methyloct-2-yl)phenolate.

460. The composition of any one of claims 1-19, wherein the molecule is damnacanthal, which has CAS Registry Number 477-84-9; and the anion is 3-formyl-4-methoxy-9,10-dioxoanthracene-2-oxide.

461. The composition of any one of claims 1-19, wherein the molecule is DB-2073, which has CAS Registry Number 39341-78-1; and the anion is 2-hexyl-3-hydroxy-5-propylphenolate.

462. The composition of any one of claims 1-19, wherein the molecule is debromomarinone; and the anion is selected from 10-hydroxy-2,5-dimethyl-7,12-dioxo-5-(4-methylpent-3-enyl)-3,4,4a,12b-tetrahydrodibenzo[c,g]chromene-8-oxide and 8-hydroxy-2,5-dimethyl-7,12-dioxo-5-(4-methylpent-3-enyl)-3,4,4a,12b-tetrahydrodibenzo[c,g]chromene-10-oxide.

463. The composition of any one of claims 1-19, wherein the molecule is delta-tocotrienol, which has CAS Registry Number 25612-59-3; and the anion is 2,8-dimethyl-2-(4,8,12-trimethyltrideca-3,7,11-trieneyl)-3,4-dihydro-2H-chromene-6-oxide.

464. The composition of any one of claims 1-19, wherein the molecule is demethoxycurcumin, which has CAS Registry Number 22608-11-3; and the anion is selected from 4-[7-(4-hydroxy-3-methoxyphenyl)-3,5-dioxohepta-1,6-dieneyl]phenolate and 2-methoxy-4-[7-(4-hydroxyphenyl)-3,5-dioxohepta-1,6-dieneyl]phenolate.

465. The composition of any one of claims 1-19, wherein the molecule is derrubone, which has CAS Registry Number 22044-58-2; and the anion is selected from 7-hydroxy-6-isoprenyl-4-oxo-3-(1,3-benzodioxol-5-yl)-4H-chromene-5-oxide and 5-hydroxy-6-isoprenyl-4-oxo-3-(1,3-benzodioxol-5-yl)-4H-chromene-7-oxide.

466. The composition of any one of claims 1-19, wherein the molecule is dianol; and the anion is selected from 4-[3-(4-hydroxyphenyl)-2-methylpent-1-enyl]phenolate and 4-[1-(4-hydroxyphenyl)-2-methylpent-1-en-3-yl]phenolate.

467. The composition of any one of claims 1-19, wherein the molecule is dihydrokanakugiol, which has CAS Registry Number 117842-21-4; and the anion is 3,4,5,6-tetramethoxy-2-(3-phenyl-1-oxopropyl)phenolate.

468. The composition of any one of claims 1-19, wherein the molecule is dihydromorin, which has CAS Registry Number 18422-83-8; and the anion is selected from 3,7-dihydroxy-4-oxo-2-(2,4-dihydroxyphenyl)-2,3-dihydro-4H-chromene-5-oxide; 3,5-dihydroxy-4-oxo-2-(2,4-dihydroxyphenyl)-2,3-dihydro-4H-chromene-7-oxide; 3-hydroxy-4-(3,5,7-trihydroxy-4-oxo-2,3-dihydro-4H-chromen-2-yl)phenolate; and 3-hydroxy-6-(3,5,7-trihydroxy-4-oxo-2,3-dihydro-4H-chromen-2-yl)phenolate.

469. The composition of any one of claims 1-19, wherein the molecule is dimethylheptylpyran, which has CAS Registry Number 32904-22-6; and the anion is 6,6,9-trimethyl-3-(3-methyloct-2-yl)-7,8,9,10-tetrahydro-6H-benzo[c]chromene-1-oxide.

470. The composition of any one of claims 1-19, wherein the molecule is diosmetin, which has CAS Registry Number 520-34-3; and the anion is selected from 7-hydroxy-4-oxo-2-(3-hydroxy-4-methoxyphenyl)-4H-chromene-5-oxide; 5-hydroxy-4-oxo-2-(3-hydroxy-4-methoxyphenyl)-4H-chromene-7-oxide; and 2-methoxy-5-(5,7-dihydroxy-4-oxo-4H-chromen-2-yl)phenolate.

471. The composition of any one of claims 1-19, wherein the molecule is DOPAL, which has CAS Registry Number 5707-55-1; and the anion is selected from 2-hydroxy-4-(2-oxoethyl)phenolate and 2-hydroxy-5-(2-oxoethyl)phenolate.

472. The composition of any one of claims 1-19, wherein the molecule is DOPEG, which has CAS Registry Number 3343-19-9; and the anion is selected from 2-hydroxy-4-(1,2-dihydroxyethyl)phenolate and 2-hydroxy-5-(1,2-dihydroxyethyl)phenolate.

473. The composition of any one of claims 1-19, wherein the molecule is drupanol, which has CAS Registry Number 42041-17-8; and the anion is 4-(4-ethenyl-4,7-dimethyl-1,6-octadien-2-yl)phenolate.

474. The composition of any one of claims 1-19, wherein the molecule is durantin A; and the anion is 2-methoxy-4-(5-methoxy-9-oxo-2-{[(acetyl)oxy]methyl}-2,3-dihydropyrano[3,2-h][1,4]benzodioxin-3-yl)phenolate.

475. The composition of any one of claims 1-19, wherein the molecule is echioidinin, which has CAS Registry Number 4308-56-9; and the anion is selected from 7-methoxy-4-oxo-2-(2-hydroxyphenyl)-4H-chromene-5-oxide and 2-(5-hydroxy-7-methoxy-4-oxo-4H-chromen-2-yl)phenolate.

476. The composition of any one of claims 1-19, wherein the molecule is enterodiol, which has CAS Registry Number 80226-00-2; and the anion is 3-[2,3-bis(hydroxymethyl)-4-(3-hydroxyphenyl)butyl]phenolate.

477. The composition of any one of claims 1-19, wherein the molecule is epicatechin gallate, which has CAS Registry Number 1257-08-5; and the anion is selected from 2,3-dihydroxy-5-({[5,7-dihydroxy-2-(3,4-dihydroxyphenyl)-3,4-dihydro-2H-chromen-3-yl]oxy}carbonyl)phenolate; 2,6-dihydroxy-4-({[5,7-dihydroxy-2-(3,4-dihydroxyphenyl)-3,4-dihydro-2H-chromen-3-yl]oxy}carbonyl)phenolate; 7-hydroxy-2-(3,4-dihydroxyphenyl)-3-{[(3,4,5-trihydroxyphenyl)carbonyl]oxy}-3,4-dihydro-2H-chromene-5-oxide; 5-hydroxy-2-(3,4-dihydroxyphenyl)-3-{[(3,4,5-trihydroxyphenyl)carbonyl]oxy}-3,4-dihydro-2H-chromene-7-oxide; 2-hydroxy-4-(5,7-dihydroxy-3-f{[(3,4,5-trihydroxyphenyl)carbonyl]oxy}-3,4-dihydro-2H-chromen-2-yl)phenolate; and 2-hydroxy-5-(5,7-dihydroxy-3-{[(3,4,5-trihydroxyphenyl)carbonyl]oxy}-3,4-dihydro-2H-chromen-2-yl)phenolate.

478. The composition of any one of claims 1-19, wherein the molecule is ermanin, which has CAS Registry Number 20869-95-8; and the anion is selected from 7-hydroxy-3-methoxy-4-oxo-2-(4-methoxyphenyl)-4H-chromene-5-oxide and 5-hydroxy-3-methoxy-4-oxo-2-(4-methoxyphenyl)-4H-chromene-7-oxide.

479. The composition of any one of claims 1-19, wherein the molecule is estetrol, which has CAS Registry Number 15183-37-6; and the anion is 15,16,17-trihydroxy-13-methyl-6,7,8,9,11,12,14,15,16,17-decahydrocyclopenta[a]phenanthrene-3-oxide.

480. The composition of any one of claims 1-19, wherein the molecule is eupatilin, which has CAS Registry Number 22368-21-4; and the anion is selected from 7-hydroxy-6-methoxy-4-oxo-2-(3,4-dimethoxyphenyl)-4H-chromene-5-oxide and 5-hydroxy-6-methoxy-4-oxo-2-(3,4-dimethoxyphenyl)-4H-chromene-7-oxide.

481. The composition of any one of claims 1-19, wherein the molecule is euxanthone, which has CAS Registry Number 529-61-3; and the anion is selected from 1-hydroxy-9-oxoxanthene-7-oxide and 8-hydroxy-9-oxoxanthene-2-oxide.

482. The composition of any one of claims 1-19, wherein the molecule is ferruginol, which has CAS Registry Number 514-62-5; and the anion is 4b,8,8-trimethyl-2-isopropyl-5,6,7,8a,9,10-hexahydrophenanthren-3-oxide.

483. The composition of any one of claims 1-19, wherein the molecule is ferujol, which has CAS Registry Number 98299-78-6; and the anion is 8-[(3,7-dimethyloct-2-enyl)oxy]-2-oxo-2H-chromene-7-oxide.

484. The composition of any one of claims 1-19, wherein the molecule is fisetinidol, which has CAS Registry Number 490-49-3; and the anion is selected from 3-hydroxy-2-(3,4-dihydroxyphenyl)-3,4-dihydro-2H-chromene-7-oxide; 2-hydroxy-4-(3,7-dihydroxy-3,4-dihydro-2H-chromen-2-yl)phenolate; and 2-hydroxy-5-(3,7-dihydroxy-3,4-dihydro-2H-chromen-2-yl)phenolate.

485. The composition of any one of claims 1-19, wherein the molecule is gamma-tocotrienol, which has CAS Registry Number 14101-61-2; and the anion is 2,7,8-trimethyl-2-(4,8,12-trimethyltrideca-3,7,11-trieneyl)-3,4-dihydro-2H-chromene-6-oxide.

486. The composition of any one of claims 1-19, wherein the molecule is garbanzol, which has CAS Registry Number 1226-22-8; and the anion is selected from 3-hydroxy-4-oxo-2-(4-hydroxyphenyl)-2,3-dihydro-4H-chromene-7-oxide and 4-(3,7-dihydroxy-4-oxo-2,3-dihydro-4H-chromen-2-yl)phenolate.

487. The composition of any one of claims 1-19, wherein the molecule is genkwanin, which has CAS Registry Number 437-64-9; and the anion is selected from 7-methoxy-4-oxo-2-(4-hydroxyphenyl)-4H-chromene-5-oxide and 4-(5-hydroxy-7-methoxy-4-oxo-4H-chromen-2-yl)phenolate.

488. The composition of any one of claims 1-19, wherein the molecule is geraldone, which has CAS Registry Number 21583-32-4; and the anion is selected from 4-oxo-2-(4-hydroxy-3-methoxyphenyl)-4H-chromene-7-oxide and 2-methoxy-4-(7-hydroxy-4-oxo-4H-chromen-2-yl)phenolate.

489. The composition of any one of claims 1-19, wherein the molecule is geranin A, which has CAS Registry Number 226715-59-9; and the anion is selected from 4-[6,9,17,19,21-pentahydroxy-13-(4-hydroxyphenyl)-4,12,14-trioxapentacyclo[11.7.1.0^{2,11}.0^{3,8}.0^{15,20}]henicosa-2(11),3(8),9,15,17,19-hexaen-5-yl]phenolate; 4-[6,9,17,19,21-pentahydroxy-5-(4-hydroxyphenyl)-4,12,14-trioxapentacyclo[11.7.1.0^{2,11}.0^{3,8}.0^{15,20}]henicosa-2(11),3(8),9,15,17,19-hexaen-13-yl]phenolate; 6,17,19,21-tetrahydroxy-5,13-bis(4-hydroxyphenyl)-4,12,14-trioxapentacyclo[11.7.1.0^{2,11}.0^{3,8}.0^{15,20}]henicosa-2(11),3(8),9,15,17,19-hexaene-9-oxide; 6,9,19,21-tetrahydroxy-5,13-bis(4-hydroxyphenyl)-4,12,14-trioxapentacyclo[11.7.1.0^{2,11}.0^{3,8}.0^{15,20}]henicosa-2(11),3(8),9,15,17,19-hexaene-17-oxide; and 6,9,17,21-tetrahydroxy-5,13-bis(4-hydroxyphenyl)-4,12,14-trioxapentacyclo[11.7.1.0^{2,11}.0^{3,8}.0^{15,20}]henicosa-2(11),3(8),9,15,17,19-hexaene-19-oxide.

490. The composition of any one of claims 1-19, wherein the molecule is glabrene, which has CAS Registry Number 60008-03-9; and the anion is selected from 3-(5-hydroxy-2,2-dimethyl-2H-chromen-8-yl)-2H-chromene-7-oxide and 2,2-dimethyl-8-(7-hydroxy-2H-chromen-3-yl)-2H-chromene-5-oxide.

491. The composition of any one of claims 1-19, wherein the molecule is glabridin, which has CAS Registry Number 59870-68-7; and the anion is selected from 3-hydroxy-4-[8,8-dimethyl-3,4-dihydro-2H-pyrano[2,3-f]chromen-3-yl]phenolate and 3-hydroxy-6-[8,8-dimethyl-3,4-dihydro-2H-pyrano[2,3-f]chromen-3-yl]phenolate.

492. The composition of any one of claims 1-19, wherein the molecule is glyceollin I, which has CAS Registry Number 57103-57-8; and the anion is 6a-hydroxy-2,2-dimethyl-6,11a-dihydroxy-[1]benzofuro[3,2-c]pyrano[2,3-h]chromene-9-oxide.

493. The composition of any one of claims 1-19, wherein the molecule is glyceollin II, which has CAS Registry Number 67314-98-1; and the anion is 7a-hydroxy-3,3-dimethyl-6,12a-dihydro-[1]benzofuro[3,2-c]pyrano[3,2-g]chromene-10-oxide.

494. The composition of any one of claims 1-19, wherein the molecule is glyceollin III, which has CAS Registry Number 61080-23-7; and the anion is 6a-hydroxy-2-isopropenyl-1,2,6,11a-tetrahydro-[1]benzofuro[3,2-c]furo[3,2-g]chromene-9-oxide.

495. The composition of any one of claims 1-19, wherein the molecule is glyceollin IV, which has CAS Registry Number 69393-94-8; and the anion is 6a-hydroxy-2-isoprenyl-3-methoxy-6,11a-dihydro-[1]benzofuro[3,2-c]chromene-9-oxide.

496. The composition of any one of claims 1-19, wherein the molecule is glycinol, which has CAS Registry Number 69393-95-9; and the anion is selected from 3,6a-dihydroxy-6,11a-dihydro-[1]benzofuro[3,2-c]chromene-9-oxide and 6a,9-dihydroxy-6,11a-dihydro-[1]benzofuro[3,2-c]chromene-3-oxide.

497. The composition of any one of claims 1-19, wherein the molecule is glycitein, which has CAS Registry Number 40957-83-3; and the anion is selected from 6-methoxy-3-(4-hydroxyphenyl)-4-oxo-4H-chromene-7-oxide and 4-(7-hydroxy-6-methoxy-4-oxo-4H-chromen-3-yl)phenolate.

498. The composition of any one of claims 1-19, wherein the molecule is glycyrrhizol A, which has CAS Registry Number 877373-00-7; and the anion is selected from 9-hydroxy-2,8-diisoprenyl-1-methoxy-6H-[1]benzofuro[3,2-c]chromene-3-oxide and 3-hydroxy-2,8-diisoprenyl-1-methoxy-6H-[1]benzofuro[3,2-c]chromene-9-oxide.

499. The composition of any one of claims 1-19, wherein the molecule is gossypetin, which has CAS Registry Number 489-35-0; and the anion is selected from 5,7,8-trihydroxy-4-oxo-2-(3,4-dihydroxyphenyl)-4H-chromene-3-oxide; 3,7,8-trihydroxy-4-oxo-2-(3,4-dihydroxyphenyl)-4H-chromene-5-oxide; 3,5,8-trihydroxy-4-oxo-2-(3,4-dihydroxyphenyl)-4H-chromene-7-oxide; 3,5,7-trihydroxy-4-oxo-2-(3,4-dihydroxyphenyl)-4H-chromene-8-oxide; 2-hydroxy-4-(3,5,7,8-tetrahydroxy-4-oxo-4H-chromen-2-yl)phenolate; and 2-hydroxy-5-(3,5,7,8-tetrahydroxy-4-oxo-4H-chromen-2-yl)phenolate.

500. The composition of any one of claims 1-19, wherein the molecule is guibourtinidol; and the anion is selected from 3-hydroxy-2-(4-hydroxyphenyl)-3,4-dihydro-2H-chromene-7-oxide and 4-(3,7-dihydroxy-3,4-dihydro-2H-chromen-2-yl)phenolate.

501. The composition of any one of claims 1-19, wherein the molecule is heptylparaben, which has CAS Registry Number 1085-12-7; and the anion is 4-[(heptyloxy)carbonyl]phenolate.

502. The composition of any one of claims 1-19, wherein the molecule is hispidulin, which has CAS Registry Number 1447-88-7; and the anion is selected from 7-hydroxy-6-methoxy-4-oxo-2-(4-hydroxyphenyl)-4H-chromene-5-oxide; 5-hydroxy-6-methoxy-4-oxo-2-(4-hydroxyphenyl)-4H-chromene-7-oxide; and 4-(5,7-dihydroxy-6-methoxy-4-oxo-4H-chromen-2-yl)phenolate.

503. The composition of any one of claims 1-19, wherein the molecule is HU-210, which has CAS Registry Number 112830-95-2; and the anion is 9-hydroxymethyl-6,6-dimethyl-3-(2-methyloct-2-yl)-6a,7,10,10a-tetrahydro-6H-benzo[c]chromene-1-oxide.

504. The composition of any one of claims 1-19, wherein the molecule is HU-243, which has CAS Registry Number 140835-18-3; and the anion is 9-hydroxymethyl-6,6-dimethyl-3-(2-methyloct-2-yl)-6a,7,8,9,10,10a-hexahydro-6H-benzo[c]chromene-1-oxide.

505. The composition of any one of claims 1-19, wherein the molecule is HU-331, which has CAS Registry Number 137252-25-6; and the anion is 3-(6-isopropenyl-3-methylcyclohex-2-enyl)-6-pentyl-1,4-benzoquinone-2-oxide.

506. The composition of any one of claims 1-19, wherein the molecule is hydroxymatairesinol, which has CAS Registry Number 20268-71-7; and the anion is selected from 2-methoxy-4-(hydroxy-{4-oxo-5-[(4-hydroxy-3-methoxyphenyl)methyl]-3-oxacyclopentyl}methyl)phenolate and 2-methoxy-4-({2-oxo-5-[hydroxy-(4-hydroxy-3-methoxyphenyl)methyl]-3-oxacyclopentyl}methyl)phenolate.

507. The composition of any one of claims 1-19, wherein the molecule is hymenoxin, which has CAS Registry Number 56003-01-1; and the anion is selected from 7-hydroxy-6,8-dimethoxy-4-oxo-2-(3,4-dimethoxyphenyl)-4H-chromene-5-oxide and 5-hydroxy-6,8-dimethoxy-4-oxo-2-(3,4-dimethoxyphenyl)-4H-chromene-7-oxide.

508. The composition of any one of claims 1-19, wherein the molecule is hypolaetin, which has CAS Registry Number 27696-41-9; and the anion is selected from 7,8-dihydroxy-4-oxo-2-(3,4-dihydroxyphenyl)-4H-chromene-5-oxide; 5,8-dihydroxy-4-oxo-2-(3,4-dihydroxyphenyl)-4H-chromene-7-oxide; 5,7-dihydroxy-4-oxo-2-(3,4-dihydroxyphenyl)-4H-chromene-8-oxide; 2-hydroxy-4-(5,7,8-trihydroxy-4-oxo-4H-chromen-2-yl)phenolate; and 2-hydroxy-5-(5,7,8-trihydroxy-4-oxo-4H-chromen-2-yl)phenolate.

509. The composition of any one of claims 1-19, wherein the molecule is iodoresiniferatoxin, which has CAS Registry Number 535974-91-5; and the anion is 2-iodo-6-methoxy-4-(2-oxo-2-{[(13-benzyl-6-hydroxy-4,17-dimethyl-5-oxo-15-isopropenyl-12,14,18-trioxapentacyclo[11.4.1.0^{1,10}.0^{2,6}.0^{11,15}]octadeca-3,8-dien-8-yl)methyl]oxy}ethyl)phenolate.

510. The composition of any one of claims 1-19, wherein the molecule is irilone, which has CAS Registry Number 41653-81-0; and the anion is selected from 8-oxo-7-(4-hydroxyphenyl)-[1,3]diolxolo[4,5-g]chromen-9-oxide and 4-(9-hydroxy-8-oxo-[1,3]diolxolo[4,5-g]chromen-7-yl)phenolate.

511. The composition of any one of claims 1-19, wherein the molecule is isojacareubin, which has CAS Registry Number 50597-93-8; and the anion is selected from 10,11-dihydroxy-3,3-dimethyl-7-oxopyrano[2,3-c]xanthen-6-oxide; 6,11-dihydroxy-3,3-dimethyl-7-oxopyrano[2,3-c]xanthen-10-oxide; and 6,10-dihydroxy-3,3-dimethyl-7-oxopyrano[2,3-c]xanthen-11-oxide.

512. The composition of any one of claims 1-19, wherein the molecule is isoliquiritigenin, which has CAS Registry Number 961-29-5; and the anion is selected from [3-oxo-3-(2,4-dihydroxyphenyl)prop-1-enyl]phenolate; 3-hydroxy-4-[1-oxo-3-(4-hydroxyphenyl)prop-2-enyl]phenolate; and 3-hydroxy-6-[1-oxo-3-(4-hydroxyphenyl)prop-2-enyl]phenolate.

513. The composition of any one of claims 1-19, wherein the molecule is isorhamnetin, which has CAS Registry Number 480-19-3; and the anion is selected from 5,7-dihydroxy-4-oxo-2-(4-hydroxy-3-methoxyphenyl)-4H-chromene-3-oxide; 3,7-dihydroxy-4-oxo-2-(4-hydroxy-3-methoxyphenyl)-4H-chromene-5-oxide; 3,5-dihydroxy-4-oxo-2-(4-hydroxy-3-methoxyphenyl)-4H-chromene-7-oxide; and 2-methoxy-4-(3,5,7-trihydroxy-4-oxo-4H-chromen-2-yl)phenolate.

514. The composition of any one of claims 1-19, wherein the molecule is isosakuranetin, which has CAS Registry Number 480-43-3; and the anion is selected from 7-hydroxy-4-oxo-2-(4-methoxyphenyl)-2,3-dihydro-4H-chromene-5-oxide and 5-hydroxy-4-oxo-2-(4-methoxyphenyl)-2,3-dihydro-4H-chromene-7-oxide.

515. The composition of any one of claims 1-19, wherein the molecule is isoscutellarein, which has CAS Registry Number 41440-05-5; and the anion is selected from 7,8-dihydroxy-4-oxo-2-(4-hydroxyphenyl)-4H-chromene-5-oxide; 5,8-dihydroxy-4-oxo-2-(4-hydroxyphenyl)-4H-chromene-7-oxide; 5,7-dihydroxy-4-oxo-2-(4-hydroxyphenyl)-4H-chromene-8-oxide; and 4-(5,7,8-trihydroxy-4-oxo-4H-chromen-2-yl)phenolate.

516. The composition of any one of claims 1-19, wherein the molecule is isosilychristin, which has CAS Registry Number 77182-66-2; and the anion is selected from 3,7-dihydroxy-4-oxo-2-[7-hydroxy-3-(hydroxymethyl)-2-(4-hydroxy-3-methoxyphenyl)-2,3-dihydro-1-benzofuran-4-yl]-2,3-dihydro-4H-chromene-5-oxide; 3,5-dihydroxy-4-oxo-2-[7-hydroxy-3-(hydroxymethyl)-2-(4-hydroxy-3-methoxyphenyl)-2,3-dihydro-1-benzofuran-4-yl]-2,3-dihydro-4H-chromene-7-oxide; 2-methoxy-4-[7-hydroxy-3-(hydroxymethyl)-4-(3,5,7-trihydroxy-4-oxo-2,3-dihydro-4H-chromen-2-yl)-2,3-dihydro-1-benzofuran-2-yl]phenolate; and 3-(hydroxymethyl)-2-(4-hydroxy-3-methoxyphenyl)-4-(3,5,7-trihydroxy-4-oxo-2,3-dihydro-4H-chromen-2-yl)-1-benzofuran-7-oxide.

517. The composition of any one of claims 1-19, wherein the molecule is isoxanthohumol, which has CAS Registry Number 521-48-2; and the anion is selected from 8-isoprenyl-5-methoxy-4-oxo-2-(4-hydroxyphenyl)-2,3-dihydro-4H-chromene-7-oxide and 4-(8-isoprenyl-7-hydroxy-5-methoxy-4-oxo-2,3-dihydro-4H-chromen-2-yl)phenolate.

518. The composition of any one of claims 1-19, wherein the molecule is jaceosidin, which has CAS Registry Number 18085-97-7; and the anion is selected from 7-hydroxy-6-methoxy-4-oxo-2-(4-hydroxy-3-methoxyphenyl)-4H-chromene-5-oxide; 5-hydroxy-6-methoxy-4-oxo-2-(4-hydroxy-3-methoxyphenyl)-4H-chromene-7-oxide; and 2-methoxy-4-(5,7-dihydroxy-6-methoxy-4-oxo-4H-chromen-2-yl)phenolate.

519. The composition of any one of claims 1-19, wherein the molecule is kanakugiol, which has CAS Registry Number 50489-48-0; and the anion is 2,3,4,5-tetramethoxy-6-(1-oxo-3-phenylprop-2-enyl)phenolate.

520. The composition of any one of claims 1-19, wherein the molecule is KM-233, which has CAS Registry Number 628263-22-9; and the anion is 6,6,9-trimethyl-3-(2-phenylprop-2-yl)-6a,7,10,10a-tetrahydro-6H-benzo[c]chromene-1-oxide.

521. The composition of any one of claims 1-19, wherein the molecule is kuwanon G, which has CAS Registry Number 75629-19-5; and the anion is selected from 3-hydroxy-4-(3-isoprenyl-5,7-dihydroxy-4-oxo-8-{3-methyl-6-[(2,4-dihydroxyphenyl)carbonyl]-5-(2,4-dihydroxyphenyl)cyclohex-2-enyl]-4H-chromen-2-yl)phenolate; 3-hydroxy-6-(3-isoprenyl-5,7-dihydroxy-4-oxo-8-{3-methyl-6-[(2,4-dihydroxyphenyl)carbonyl]-5-(2,4-dihydroxyphenyl)cyclohex-2-enyl]-4H-chromen-2-yl)phenolate; 3-isoprenyl-7-hydroxy-4-oxo-2-(2,4-dihydroxyphenyl)-8-{3-methyl-6-[(2,4-dihydroxyphenyl)carbonyl]-5-(2,4-dihydroxyphenyl)cyclohex-2-enyl}-4H-chromene-5-oxide; 3-isoprenyl-5-hydroxy-4-oxo-2-(2,4-dihydroxyphenyl)-8-{3-methyl-6-[(2,4-dihydroxyphenyl)carbonyl]-5-(2,4-dihydroxyphenyl)cyclohex-2-enyl}-4H-chromene-7-oxide; 3-hydroxy-4-{3-methyl-6-[(2,4-dihydroxyphenyl)carbonyl]-5-[3-isoprenyl-5,7-dihydroxy-4-oxo-2-(2,4-dihydroxyphenyl)-4H-chromen-8-yl]cyclohex-3-enyl}phenolate; 3-hydroxy-6-{3-methyl-6-[(2,4-dihydroxyphenyl)carbonyl]-5-[3-isoprenyl-5,7-dihydroxy-4-oxo-2-(2,4-dihydroxyphenyl)-4H-chromen-8-yl]cyclohex-3-enyl}phenolate; 3-hydroxy-4-({4-methyl-6-(2,4-dihydroxyphenyl)-2-[3-isoprenyl-5,7-dihydroxy-4-oxo-2-(2,4-dihydroxyphenyl)-4H-chromen-8-yl]cyclohex-3-enyl}carbonyl)phenolate; and 3-hydroxy-6-({4-methyl-6-(2,4-dihydroxyphenyl)-2-[3-isoprenyl-5,7-dihydroxy-4-oxo-2-(2,4-dihydroxyphenyl)-4H-chromen-8-yl]cyclohex-3-enyl}carbonyl)phenolate.

522. The composition of any one of claims 1-19, wherein the molecule is lariciresinol, which has CAS Registry Number 27003-73-2; and the anion is selected from 2-methoxy-4-{2-(hydroxymethyl)-3-[(4-hydroxy-3-methoxyphenyl)methyl]-5-oxacyclopentyl}phenolate and 2-methoxy-4-{[2-(hydroxymethyl)-3-(4-hydroxy-3-methoxyphenyl)-4-oxacyclopentyl]methyl}phenolate.

523. The composition of any one of claims 1-19, wherein the molecule is laricitrin, which has CAS Registry Number 53472-37-0; and the anion is selected from 5,7-dihydroxy-4-oxo-2-(4,5-dihydroxy-3-methoxyphenyl)-4H-chromene-3-oxide; 3,7-dihydroxy-4-oxo-2-(4,5-dihydroxy-3-methoxyphenyl)-4H-chromene-5-oxide; 3,5-dihydroxy-4-oxo-2-(4,5-dihydroxy-3-methoxyphenyl)-4H-chromene-7-oxide; 6-hydroxy-2-methoxy-4-(3,5,7-trihydroxy-4-oxo-4H-chromen-2-yl)phenolate; and 2-hydroxy-3-methoxy-5-(3,5,7-trihydroxy-4-oxo-4H-chromen-2-yl)phenolate.

524. The composition of any one of claims 1-19, wherein the molecule is leptosidin, which has CAS Registry Number 486-24-8; and the anion is selected from 7-methoxy-3-oxo-2-[(3,4-dihydroxyphenyl)methylidene]-1-benzofuran-6-oxide; 2-hydroxy-4-[(6-hydroxy-7-methoxy-3-oxo-1-benzofuran-2-ylidene)methyl]phenolate; and 2-hydroxy-5-[(6-hydroxy-7-methoxy-3-oxo-1-benzofuran-2-ylidene)methyl]phenolate.

525. The composition of any one of claims 1-19, wherein the molecule is leptosphaerin A; and the anion is 3-hydroxy-2-(4-hydroxy-5-methyl-7-oxo-5,6-dihydro-4H-2-benzofuran-1-yl)phenolate.

526. The composition of any one of claims 1-19, wherein the molecule is leucofisetinidin, which has CAS Registry Number 34620-73-0; and the anion is selected from 3,4-dihydroxy-2-(3,4-dihydroxyphenyl)-3,4-dihydro-2H-chromene-7-oxide; 2-hydroxy-4-(3,4,7-trihydroxy-3,4-dihydro-2H-chromen-2-yl)phenolate; and 2-hydroxy-5-(3,4,7-trihydroxy-3,4-dihydro-2H-chromen-2-yl)phenolate.

527. The composition of any one of claims 1-19, wherein the molecule is leucopelargonidin, which has CAS Registry Number 520-17-2; and the anion is selected from 3,4,7-trihydroxy-2-(4-hydroxyphenyl)-3,4-dihydro-2H-chromene-5-oxide; 3,4,5-trihydroxy-2-(4-hydroxyphenyl)-3,4-dihydro-2H-chromene-7-oxide; and 4-(3,4,5,7-tetrahydroxy-3,4-dihydro-2H-chromen-2-yl)phenolate.

528. The composition of any one of claims 1-19, wherein the molecule is leucopeonidin, which has CAS Registry Number 20408-92-8; and the anion is selected from 4,5,7-trihydroxy-2-(4-hydroxy-3-methoxyphenyl)-4H-chromene-3-oxide; 3,4,7-trihydroxy-2-(4-hydroxy-3-methoxyphenyl)-4H-chromene-5-oxide; 3,4,5-trihydroxy-2-(4-hydroxy-3-methoxyphenyl)-4H-chromene-7-oxide; and 2-methoxy-4-(3,4,5,7-tetrahydroxy-4H-chromen-2-yl)phenolate.

529. The composition of any one of claims 1-19, wherein the molecule is liquiritigenin, which has CAS Registry Number 578-86-9; and the anion is selected from 4-oxo-2-(4-hydroxyphenyl)-2,3-dihydro-4H-chromene-7-oxide and 4-(7-hydroxy-4-oxo-2,3-dihydro-4H-chromen-2-yl)phenolate.

530. The composition of any one of claims 1-19, wherein the molecule is luteoforol, which has CAS Registry Number 24897-98-1; and the anion is selected from 4,7-dihydroxy-2-(3,4-dihydroxyphenyl)-3,4-dihydro-2H-chromene-5-oxide; 4,5-dihydroxy-2-(3,4-dihydroxyphenyl)-3,4-dihydro-2H-chromene-7-oxide; 2-hydroxy-4-(4,5,7-trihydroxy-3,4-dihydro-2H-chromen-2-yl)phenolate; and 2-hydroxy-5-(4,5,7-trihydroxy-3,4-dihydro-2H-chromen-2-yl)phenolate.

531. The composition of any one of claims 1-19, wherein the molecule is luteone, which has CAS Registry Number 41743-56-0; and the anion is selected from 7-hydroxy-6-isoprenyl-4-oxo-3-(2,4-dihydroxyphenyl)-4H-chromene-5-oxide; 5-hydroxy-6-isoprenyl-4-oxo-3-(2,4-dihydroxyphenyl)-4H-chromene-7-oxide; 3-hydroxy-4-(5,7-dihydroxy-6-isoprenyl-4-oxo-4H-chromen-3-yl)phenolate; and 3-hydroxy-6-(5,7-dihydroxy-6-isoprenyl-4-oxo-4H-chromen-3-yl)phenolate.

532. The composition of any one of claims 1-19, wherein the molecule is macelignan, which has CAS Registry Number 107534-93-0; and the anion is 2-methoxy-4-[4-(1,3-benzodioxol-5-yl)-2,3-dimethylbutyl]phenolate.

533. The composition of any one of claims 1-19, wherein the molecule is matairesinol, which has CAS Registry Number 580-72-3; and the anion is selected from 2-methoxy-4-({3-oxo-2-[(4-hydroxy-3-methoxyphenyl)methyl]-4-oxacyclopentyl}methyl)phenolate and 2-methoxy-4-({2-oxo-5-[(4-hydroxy-3-methoxyphenyl)methyl]-3-oxacyclopentyl}methyl)phenolate.

534. The composition of any one of claims 1-19, wherein the molecule is mearnsetin, which has CAS Registry Number 16805-10-0; and the anion is selected from 5,7-dihydroxy-4-oxo-2-(3,5-dihydroxy-4-methoxyphenyl)-4H-chromene-3-oxide; 3,7-dihydroxy-4-oxo-2-(3,5-dihydroxy-4-methoxyphenyl)-4H-chromene-5-oxide; 3,5-dihydroxy-4-oxo-2-(3,5-dihydroxy-4-methoxyphenyl)-4H-chromene-7-oxide; and 3-hydroxy-2-methoxy-5-(3,5,7-trihydroxy-4-oxo-4H-chromen-2-yl)phenolate.

535. The composition of any one of claims 1-19, wherein the molecule is meciadanol, which has CAS Registry Number 65350-86-9; and the anion is selected from 7-hydroxy-3-methoxy-2-(3,4-dihydroxyphenyl)-3,4-dihydro-2H-chromene-5-oxide; 5-hydroxy-3-methoxy-2-(3,4-dihydroxyphenyl)-3,4-dihydro-2H-chromene-7-oxide; 2-hydroxy-4-(5,7-dihydroxy-3-methoxy-3,4-dihydro-2H-chromen-2-yl)phenolate; and 2-hydroxy-5-(5,7-dihydroxy-3-methoxy-3,4-dihydro-2H-chromen-2-yl)phenolate.

536. The composition of any one of claims 1-19, wherein the molecule is melacacidin, which has CAS Registry Number 38081-16-2; and the anion is selected from 3,4,8-trihydroxy-2-(3,4-dihydroxyphenyl)-3,4-dihydro-2H-chromene-7-oxide; 3,4,7-trihydroxy-2-(3,4-dihydroxyphenyl)-3,4-dihydro-2H-chromene-8-oxide; 2-hydroxy-4-(3,4,7,8-tetrahydroxy-3,4-dihydro-2H-chromen-2-yl)phenolate; and 2-hydroxy-5-(3,4,7,8-tetrahydroxy-3,4-dihydro-2H-chromen-2-yl)phenolate.

537. The composition of any one of claims 1-19, wherein the molecule is mequinol, which has CAS Registry Number 150-76-5; and the anion is 4-methoxyphenolate.

538. The composition of any one of claims 1-19, wherein the molecule is mesquitol, which has CAS Registry Number 109671-55-8; and the anion is selected from 3,8-dihydroxy-2-(3,4-dihydroxyphenyl)-3,4-dihydro-2H-chromene-7-oxide; 3,7-dihydroxy-2-(3,4-dihydroxyphenyl)-3,4-dihydro-2H-chromene-8-oxide; 2-hydroxy-4-(3,7,8-trihydroxy-3,4-dihydro-2H-chromen-2-yl)phenolate; and 2-hydroxy-5-(3,7,8-trihydroxy-3,4-dihydro-2H-chromen-2-yl)phenolate.

539. The composition of any one of claims 1-19, wherein the molecule is mikanin, which has CAS Registry Number 4324-53-2; and the anion is selected from 5-hydroxy-6,7-dimethoxy-4-oxo-2-(4-methoxyphenyl)-4H-chromene-3-oxide and 3-hydroxy-6,7-dimethoxy-4-oxo-2-(4-methoxyphenyl)-4H-chromene-5-oxide.

540. The composition of any one of claims 1-19, wherein the molecule is mutisianthol, which has CAS Registry Number 70855-59-3; and the anion is 3,6-dimethyl-1-(2-methylprop-1-enyl)-2,3-dihydro-1H-indene-5-oxide.

541. The composition of any one of claims 1-19, wherein the molecule is myricanone, which has CAS Registry Number 32492-74-3; and the anion is selected from 15-hydroxy-16,17-dimethoxy-9-oxotricyclo[12.3.1.1^{2,6}]nonadeca-1(17),2,4,6(19),14(18),15-hexaen-3-oxide and 3-hydroxy-16,17-dimethoxy-9-oxotricyclo[12.3.1.1^{2,6}]nonadeca-1(17),2,4,6(19),14(18),15-hexaen-15-oxide.

542. The composition of any one of claims 1-19, wherein the molecule is naphthazarin, which has CAS Registry Number 475-38-7; and the anion is 4-hydroxy-5,8-dioxonaphthalene-1-oxide.

543. The composition of any one of claims 1-19, wherein the molecule is negletein, which has CAS Registry Number 29550-13-8; and the anion is selected from 6-hydroxy-7-methoxy-4-oxo-2-phenyl-4H-chromene-5-oxide and 5-hydroxy-7-methoxy-4-oxo-2-phenyl-4H-chromene-6-oxide.

544. The composition of any one of claims 1-19, wherein the molecule is nepetin, which has CAS Registry Number 520-11-6; and the anion is selected from 7-hydroxy-6-methoxy-4-oxo-2-(3,4-dihydroxyphenyl)-4H-chromene-5-oxide; 5-hydroxy-6-methoxy-4-oxo-2-(3,4-dihydroxyphenyl)-4H-chromene-7-oxide; 2-hydroxy-4-(5,7-dihydroxy-6-methoxy-4-oxo-4H-chromen-2-yl)phenolate; and 2-hydroxy-5-(5,7-dihydroxy-6-methoxy-4-oxo-4H-chromen-2-yl)phenolate.

545. The composition of any one of claims 1-19, wherein the molecule is nevadensin, which has CAS Registry Number 10176-66-6; and the anion is selected from 7-hydroxy-6,8-dimethoxy-4-oxo-2-(4-methoxyphenyl)-4H-chromene-5-oxide and 5-hydroxy-6,8-dimethoxy-4-oxo-2-(4-methoxyphenyl)-4H-chromene-7-oxide.

546. The composition of any one of claims 1-19, wherein the molecule is nigrosporin B; and the anion is 6,7-dihydroxy-3-methoxy-1,4-dioxo-6-methyl-7,8-dihydro-5H-anthracene-9-oxide.

547. The composition of any one of claims 1-19, wherein the molecule is nodifloretin, which has CAS Registry Number 23494-48-6; and the anion is selected from 6,7-dihydroxy-4-oxo-2-(4-hydroxy-3-methoxyphenyl)-4H-chromene-5-oxide; 5,7-dihydroxy-4-oxo-2-(4-hydroxy-3-methoxyphenyl)-4H-chromene-6-oxide; 5,6-dihydroxy-4-oxo-2-(4-hydroxy-3-methoxyphenyl)-4H-chromene-7-oxide; and 2-methoxy-4-(5,6,7-trihydroxy-4-oxo-4H-chromen-2-yl)phenolate.

548. The composition of any one of claims 1-19, wherein the molecule is norartocarpetin, which has CAS Registry Number 520-30-9; and the anion is selected from 7-hydroxy-4-oxo-2-(2,4-dihydroxyphenyl)-4H-chromen-5-oxide; 5-hydroxy-4-oxo-2-(2,4-dihydroxyphenyl)-4H-chromen-7-oxide; 3-hydroxy-4-(5,7-dihydroxy-4-oxo-4H-chromen-2-yl)phenolate; and 3-hydroxy-6-(5,7-dihydroxy-4-oxo-4H-chromen-2-yl)phenolate.

549. The composition of any one of claims 1-19, wherein the molecule is norwogonin, which has CAS Registry Number 4443-09-8; and the anion is selected from 7,8-dihydroxy-4-oxo-2-phenyl-4H-chromene-5-oxide; 5,8-dihydroxy-4-oxo-2-phenyl-4H-chromene-7-oxide; and 5,7-dihydroxy-4-oxo-2-phenyl-4H-chromene-8-oxide.

550. The composition of any one of claims 1-19, wherein the molecule is 0-1602, which has CAS Registry Number 317321-41-8; and the anion is 3-hydroxy-5-methyl-4-(3-methyl-6-isopropenylcyclohex-2-enyl]phenolate.

551. The composition of any one of claims 1-19, wherein the molecule is 0-1656; and the anion is 2-cycloheptyl-3-hydroxy-5-(2-methyloct-2-yl)phenolate.

552. The composition of any one of claims 1-19, wherein the molecule is 0-1660; and the anion is 2-adamantyl-3-hydroxy-5-(2-methyloct-2-yl)phenolate.

553. The composition of any one of claims 1-19, wherein the molecule is 0-1871, which has CAS Registry Number 620964-96-7; and the anion is 3-hydroxy-2-(3,3-dimethylcyclohexyl)-5-(2-methyloct-2-yl)phenolate.

554. The composition of any one of claims 1-19, wherein the molecule is 0-806; and the anion is 6,6,9-trimethyl-3-(6-bromohex-2-ynyl)-6a,7,10,10a-tetrahydro-6H-benzo[c]chromene-1-oxide.

555. The composition of any one of claims 1-19, wherein the molecule is obovatol, which has CAS Registry Number 83864-78-2; and the anion is selected from 2-hydroxy-5-(prop-2-enyl)-3-[4-(prop-2-enyl)phenoxy]phenolate and 2-hydroxy-4-(prop-2-enyl)-6-[4-(prop-2-enyl)phenoxy]phenolate.

556. The composition of any one of claims 1-19, wherein the molecule is olivetol, which has CAS Registry Number 500-66-3; and the anion is 3-hydroxy-5-pentylphenolate.

557. The composition of any one of claims 1-19, wherein the molecule is onopordin; and the anion is selected from 7-hydroxy-8-methoxy-4-oxo-2-(3,4-dihydroxyphenyl)-4H-chromene-5-oxide; 5-hydroxy-8-methoxy-4-oxo-2-(3,4-dihydroxyphenyl)-4H-chromene-7-oxide; 2-hydroxy-4-(5,7-dihydroxy-8-methoxy-4-oxo-4H-chromen-2-yl)phenolate; and 2-hydroxy-5-(5,7-dihydroxy-8-methoxy-4-oxo-4H-chromen-2-yl)phenolate.

558. The composition of any one of claims 1-19, wherein the molecule is oritin; and the anion is selected from 3,8-dihydroxy-2-(4-hydroxyphenyl)-3,4-dihydro-2H-chromene-7-oxide; 3,7-dihydroxy-2-(4-hydroxyphenyl)-3,4-dihydro-2H-chromene-8-oxide; and 4-(3,7,8-trihydroxy-3,4-dihydro-2H-chromen-2-yl)phenolate.

559. The composition of any one of claims 1-19, wherein the molecule is orobol, which has CAS Registry Number 480-23-9; and the anion is selected from 7-hydroxy-4-oxo-3-(3,4-dihydroxyphenyl)-4H-chromene-5-oxide; 5-hydroxy-4-oxo-3-(3,4-dihydroxyphenyl)-4H-chromene-7-oxide; 2-hydroxy-4-(5,7-dihydroxy-4-oxo-4H-chromen-3-yl)phenolate; and 2-hydroxy-5-(5,7-dihydroxy-4-oxo-4H-chromen-3-yl)phenolate.

560. The composition of any one of claims 1-19, wherein the molecule is ortho-phenylphenol, which has CAS Registry Number 90-43-7; and the anion is 2-phenylphenolate.

561. The composition of any one of claims 1-19, wherein the molecule is oxyanthrarufin, which has CAS Registry Number 6486-93-7; and the anion is selected from 2,5-dihydroxy-9,10-dioxoanthracene-1-oxide; 1,5-dihydroxy-9,10-dioxoanthracene-2-oxide; and 5,6-dihydroxy-9,10-dioxoanthracene-1-oxide.

562. The composition of any one of claims 1-19, wherein the molecule is oxychrysazin, which has CAS Registry Number 51030-24-1; and the anion is selected from 2,8-dihydroxy-9,10-dioxoanthracene-1-oxide; 1,8-dihydroxy-9,10-dioxo-anthracene-2-oxide; and 7,8-dihydroxy-9,10-dioxo-anthracene-1-oxide.

563. The composition of any one of claims 1-19, wherein the molecule is pachypodol, which has CAS Registry Number 33708-72-4; and the anion is selected from 3,7-dimethoxy-4-oxo-2-(4-hydroxy-3-methoxyphenyl)-4H-chromene-5-oxide and 2-methoxy-4-(5-hydroxy-3,7-dimethoxy-4-oxo-4H-chromen-2-yl)phenolate.

564. The composition of any one of claims 1-19, wherein the molecule is patuletin, which has CAS Registry Number 519-96-0; and the anion is selected from 5,7-dihydroxy-6-methoxy-4-oxo-2-(3,4-dihydroxyphenyl)-4H-chromene-3-oxide; 3,7-dihydroxy-6-methoxy-4-oxo-2-(3,4-dihydroxyphenyl)-4H-chromene-5-oxide; 3,5-dihydroxy-6-methoxy-4-oxo-2-(3,4-dihydroxyphenyl)-4H-chromene-7-oxide; 2-hydroxy-4-(3,5,7-trihydroxy-6-methoxy-4-oxo-4H-chromen-2-yl)phenolate; and 2-hydroxy-5-(3,5,7-trihydroxy-6-methoxy-4-oxo-4H-chromen-2-yl)phenolate.

565. The composition of any one of claims 1-19, wherein the molecule is pedalitin, which has CAS Registry Number 22384-63-0; and the anion is selected from 6-hydroxy-7-methoxy-4-oxo-2-(3,4-dihydroxyphenyl)-4H-chromene-5-oxide; 5-hydroxy-7-methoxy-4-oxo-2-(3,4-dihydroxyphenyl)-4H-chromene-6-oxide; 2-hydroxy-4-(5,6-dihydroxy-7-methoxy-4-oxo-4H-chromen-2-yl)phenolate; and 2-hydroxy-5-(5,6-dihydroxy-7-methoxy-4-oxo-4H-chromen-2-yl)phenolate.

566. The composition of any one of claims 1-19, wherein the molecule is perrottetinene, which has CAS Registry Number 160041-34-9; and the anion is 6,6,9-trimethyl-3-(2-phenylethyl)-6a,7,8,10a-tetrahydro-6H-benzo[c]chromene-1-oxide.

567. The composition of any one of claims 1-19, wherein the molecule is phenylacetylrinvanil; and the anion is 2-methoxy-4-[(N-{12-[1-oxo-2-phenyl)ethyl]octadec-9-enyl}amino)methyl]phenolate.

568. The composition of any one of claims 1-19, wherein the molecule is phylloquinol, which has CAS Registry Number 572-96-3; and the anion is selected from 4-hydroxy-2-methyl-3-(3,7,11,15-tetramethylhexadec-2-enyl)naphthalene-1-oxide and 4-hydroxy-3-methyl-2-(3,7,11,15-tetramethylhexadec-2-enyl)naphthalene-1-oxide.

569. The composition of any one of claims 1-19, wherein the molecule is piceatannol, which has CAS Registry Number 4339-71-3; and the anion is selected from 2-hydroxy-4-[2-(3,5-dihydroxyphenyl)ethenyl]phenolate; 2-hydroxy-5-[2-(3,5-dihydroxyphenyl)ethenyl]phenolate; and 3-hydroxy-5-[2-(3,4-dihydroxyphenyl)ethenyl]phenolate.

570. The composition of any one of claims 1-19, wherein the molecule is pilloin, which has CAS Registry Number 32174-62-2; and the anion is selected from 7-methoxy-4-oxo-2-(3-hydroxy-4-methoxyphenyl)-4H-chromene-5-oxide and 2-methoxy-5-(5-hydroxy-7-methoxy-4-oxo-4H-chromen-2-yl)phenolate.

571. The composition of any one of claims 1-19, wherein the molecule is pinobanksin, which has CAS Registry Number 548-82-3; and the anion is selected from 3,7-dihydroxy-4-oxo-2-phenyl-2,3-dihydro-4H-chromene-5-oxide and 3,5-dihydroxy-4-oxo-2-phenyl-2,3-dihydro-4H-chromene-7-oxide.

572. The composition of any one of claims 1-19, wherein the molecule is pinocembrin, which has CAS Registry Number 480-39-7; and the anion is selected from 7-hydroxy-4-oxo-2-phenyl-2,3-dihydro-4H-chromene-5-oxide and 5-hydroxy-4-oxo-2-phenyl-2,3-dihydro-4H-chromene-7-oxide.

573. The composition of any one of claims 1-19, wherein the molecule is pinoresinol, which has CAS Registry Number 487-36-5; and the anion is 2-methoxy-4-[6-(4-hydroxy-3-methoxyphenyl)-1,3,3a,4,6,6a-hexahydrofuro[3,4-c]furan-3-yl]phenolate.

574. The composition of any one of claims 1-19, wherein the molecule is pinostrobin, which has CAS Registry Number 480-37-5; and the anion is 7-methoxy-4-oxo-2-phenyl-2,3-dihydro-4H-chromene-5-oxide.

575. The composition of any one of claims 1-19, wherein the molecule is plicadin, which has CAS Registry Number 137551-37-2; and the anion is 2,2-dimethyl-6-oxobenzofuro[3,2-c]pyrano[2,3-h]chromene-9-oxide.

576. The composition of any one of claims 1-19, wherein the molecule is pomiferin, which has CAS Registry Number 572-03-2; and the anion is selected from 6-isoprenyl-8,8-dimethyl-4-oxo-3-(3,4-dihydroxyphenyl)pyrano[2,3-h]-4H-chromene-5-oxide; 2-hydroxy-4-(5-hydroxy-6-isoprenyl-8,8-dimethyl-4-oxopyrano[2,3-h]chromen-3-yl)phenolate; and 2-hydroxy-5-(5-hydroxy-6-isoprenyl-8,8-dimethyl-4-oxopyrano[2,3-h]chromen-3-yl)phenolate.

577. The composition of any one of claims 1-19, wherein the molecule is poriol, which has CAS Registry Number 14348-16-4; and the anion is selected from 7-hydroxy-6-methyl-4-oxo-2-(4-hydroxyphenyl)-2,3-dihydro-4H-chromene-5-oxide; 5-hydroxy-6-methyl-4-oxo-2-(4-hydroxyphenyl)-2,3-dihydro-4H-chromene-7-oxide; and 4-(5,7-dihydroxy-6-methyl-4-oxo-2,3-dihydro-4H-chromen-2-yl)phenolate.

578. The composition of any one of claims 1-19, wherein the molecule is pratol, which has CAS Registry Number 487-24-1; and the anion is 4-oxo-2-(4-methoxyphenyl)-4H-chromene-7-oxide.

579. The composition of any one of claims 1-19, wherein the molecule is primetin, which has CAS Registry Number 548-58-3; and the anion is selected from 8-hydroxy-4-oxo-2-phenyl-4H-chromene-5-oxide and 5-hydroxy-4-oxo-2-phenyl-4H-chromene-8-oxide.

580. The composition of any one of claims 1-19, wherein the molecule is primuletin, which has CAS Registry Number 491-78-1; and the anion is 4-oxo-2-phenyl-4H-chromene-5-oxide.

581. The composition of any one of claims 1-19, wherein the molecule is PSB-SB-487; and the anion is selected from 2-oxo-3-[(2-hydroxyphenyl)methyl]-7-(2-methylnonan-2-yl)-2H-chromene-5-oxide and 2-{[5-hydroxy-2-oxo-7-(2-methylnonan-2-yl)-2H-chromen-3-yl]methyl}phenolate.

582. The composition of any one of claims 1-19, wherein the molecule is pseudoisoeugenol; and the anion is 4-methoxy-2-(prop-1-enyl)phenolate.

583. The composition of any one of claims 1-19, wherein the molecule is psi-tectorigenin, which has CAS Registry Number 13111-57-4; and the anion is selected from 7-hydroxy-8-methoxy-4-oxo-3-(4-hydroxyphenyl)-4H-chromene-5-oxide; 5-hydroxy-8-methoxy-4-oxo-3-(4-hydroxyphenyl)-4H-chromene-7-oxide; and 4-(5,7-dihydroxy-8-methoxy-4-oxo-4H-chromen-3-yl)phenolate.

584. The composition of any one of claims 1-19, wherein the molecule is pterostilbene, which has CAS Registry Number 537-42-8; and the anion is 4-[2-(3,5-dimethoxyphenyl)ethenyl]phenolate.

585. The composition of any one of claims 1-19, wherein the molecule is purpuroxanthin, which has CAS Registry Number 518-83-2; and the anion is selected from 3-hydroxy-9,10-dioxoanthracene-1-oxide and 4-hydroxy-9,10-dioxoanthracene-2-oxide.

586. The composition of any one of claims 1-19, wherein the molecule is quinizarin, which has CAS Registry Number 81-64-1; and the anion is 4-hydroxy-9,10-dioxoanthracene-1-oxide.

587. The composition of any one of claims 1-19, wherein the molecule is resiniferatoxin, which has CAS Registry Number 57444-62-9; and the anion is 2-methoxy-4-(2-oxo-2-{[(13-benzyl-6-hydroxy-4,17-dimethyl-5-oxo-15-isopropenyl-12,14,18-trioxapentacyclo[11.4.1.0^{1,10}.0^{2,6}.0^{11,15}]octadeca-3,8-dien-8-yl)methyl]oxy}ethyl)phenolate.

588. The composition of any one of claims 1-19, wherein the molecule is retusin, which has CAS Registry Number 37816-19-6; and the anion is selected from 8-hydroxy-4-oxo-3-(4-methoxyphenyl)-4H-chromene-7-oxide and 7-hydroxy-4-oxo-3-(4-methoxyphenyl)-4H-chromene-8-oxide.

589. The composition of any one of claims 1-19, wherein the molecule is rhamnazin, which has CAS Registry Number 552-54-5; and the anion is selected from 5-hydroxy-7-methoxy-4-oxo-2-(4-hydroxy-3-methoxyphenyl)-4H-chromene-3-oxide; 3-hydroxy-7-methoxy-4-oxo-2-(4-hydroxy-3-methoxyphenyl)-4H-chromene-5-oxide; and 2-methoxy-4-(3,5-dihydroxy-7-methoxy-4-oxo-4H-chromen-2-yl)phenolate.

590. The composition of any one of claims 1-19, wherein the molecule is robinetinidol, which has CAS Registry Number 528-56-3; and the anion is selected from 3-hydroxy-2-(3,4,5-trihydroxyphenyl)-3,4-dihydro-2H-chromene-7-oxide; 2,3-dihydroxy-5-(3,7-dihydroxy-3,4-dihydro-2H-chromen-2-yl)phenolate; and 2,6-dihydroxy-4-(3,7-dihydroxy-3,4-dihydro-2H-chromen-2-yl)phenolate.

591. The composition of any one of claims 1-19, wherein the molecule is sakuranetin, which has CAS Registry Number 2957-21-3; and the anion is selected from 7-methoxy-4-oxo-2-(4-hydroxyphenyl)-2,3-dihydro-4H-chromene-5-oxide and 4-(5-hydroxy-7-methoxy-4-oxo-2,3-dihydro-4H-chromen-2-yl)phenolate.

592. The composition of any one of claims 1-19, wherein the molecule is sappanol, which has CAS Registry Number 111254-19-4; and the anion is selected from 3,4-dihydroxy-3-[(3,4-dihydroxyphenyl)methyl]-2,4-dihydro-3H-chromene-7-oxide; 2-hydroxy-4-[(3,4,7-trihydroxy-2,4-dihydro-3H-chromen-3-yl)methyl]phenolate; and 2-hydroxy-5-[(3,4,7-trihydroxy-2,4-dihydro-3H-chromen-3-yl)methyl]phenolate.

593. The composition of any one of claims 1-19, wherein the molecule is scaposin; and the anion is selected from 7-hydroxy-6,8-dimethoxy-4-oxo-2-(5-hydroxy-3,4-dimethoxyphenyl)-4H-chromene-5-oxide; 5-hydroxy-6,8-dimethoxy-4-oxo-2-(5-hydroxy-3,4-dimethoxyphenyl)-4H-chromene-7-oxide; and 2,3-dimethoxy-5-(5,7-dihydroxy-6,8-dimethoxy-4-oxo-4H-chromen-2-yl)phenolate.

594. The composition of any one of claims 1-19, wherein the molecule is scillavone A, which has CAS Registry Number 1105670-99-2; and the anion is selected from 5,7-dihydroxy-3′,4′-dimethoxy-4-oxospiro[2H-chromene-3,7′-bicyclo[4.2.0]octa-1,3,5-triene]-2′-oxide; 2′,7-dihydroxy-3′,4′-dimethoxy-4-oxospiro[2H-chromene-3,7′-bicyclo[4.2.0]octa-1,3,5-triene]-5-oxide; and 2′,5-dihydroxy-3′,4′-dimethoxy-4-oxospiro[2H-chromene-3,7′-bicyclo[4.2.0]octa-1,3,5-triene]-7-oxide.

595. The composition of any one of claims 1-19, wherein the molecule is scillavone B, which has CAS Registry Number 1105671-01-9; and the anion is selected from 6,7-dimethoxy-4-oxo-3-[(3,4-dihydroxyphenyl)methyl]-2,3-dihydro-4H-chromene-5-oxide; 2-hydroxy-4-[(5-hydroxy-6,7-dimethoxy-4-oxo-2,3-dihydro-4H-chromen-3-yl)methyl]phenolate; and 2-hydroxy-5-[(5-hydroxy-6,7-dimethoxy-4-oxo-2,3-dihydro-4H-chromen-3-yl)methyl]phenolate.

596. The composition of any one of claims 1-19, wherein the molecule is secoisolariciresinol, which has CAS Registry Number 29388-59-8; and the anion is 2-methoxy-4-[2,3-bis(hydroxymethyl)-4-(4-hydroxy-3-methoxyphenyl)butyl]phenolate.

597. The composition of any one of claims 1-19, wherein the molecule is selligueain A, which has CAS Registry Number 152378-18-2; and the anion is selected from 6,17,19,21-tetrahydroxy-5,13-bis(4-hydroxyphenyl)-7-[3,5,7-trihydroxy-2-(4-hydroxyphenyl)-3,4-dihydro-2H-chromen-8-yl]-4,12,14-trioxapentacyclo[11.7.1.0^{2,11}.0^{3,8}.0^{15,20}]henicosa-2(11),3(8),9,15,17,19-hexaene-9-oxide; 6,9,19,21-tetrahydroxy-5,13-bis(4-hydroxyphenyl)-7-[3,5,7-trihydroxy-2-(4-hydroxyphenyl)-3,4-dihydro-2H-chromen-8-yl]-4,12,14-trioxapentacyclo[11.7.1.02,0^{3,8}.0^{15,20}]henicosa-2(11),3(8),9,15,17,19-hexaene-17-oxide; 6,9,17,21-tetrahydroxy-5,13-bis(4-hydroxyphenyl)-7-[3,5,7-trihydroxy-2-(4-hydroxyphenyl)-3,4-dihydro-2H-chromen-8-yl]-4,12,14-trioxapentacyclo[11.7.1.0^{2,11}.0^{3,8}.0^{15,20}]henicosa-2(11),3(8),9,15,17,19-hexaene-19-oxide;
{6,9,17,19,21-pentahydroxy-13-(4-hydroxyphenyl)-7-[3,5,7-trihydroxy-2-(4-hydroxyphenyl)-3,4-dihydro-2H-chromen-8-yl]-4,12,14-trioxapentacyclo[11.7.1.0^{2,11}.0^{3,8}.0^{15,20}]henicosa-2(11),3(8),9,15,17,19-hexaen-5-yl}phenolate; {6,9,17,19,21-pentahydroxy-5-(4-hydroxyphenyl)-7-[3,5,7-trihydroxy-2-(4-hydroxyphenyl)-3,4-dihydro-2H-chromen-8-yl]-4,12,14-trioxapentacyclo[11.7.1.0^{2,11}.0^{3,8}.0^{15,20}]henicosa-2(11),3(8),9,15,17,19-hexaen-13-yl}phenolate; and 3,7-dihydroxy-2-(4-hydroxyphenyl)-8-{6,9,17,19,21-pentahydroxy-5,13-bis(4-hydroxyphenyl)-4,12,14-trioxapentacyclo[11.7.1.0^{2,11}.0^{3,8}.0^{15,20}]henicosa-2(11),3(8),9,15,17,19-hexaen-7-yl}-3,4-dihydro-2H-chromene-5-oxide.

598. The composition of any one of claims 1-19, wherein the molecule is serpyllin; and the anion is 7,8-dimethoxy-4-oxo-2-(2,3,4-trimethoxyphenyl)-4H-chromene-5-oxide.

599. The composition of any one of claims 1-19, wherein the molecule is sesamol, which has CAS Registry Number 533-31-3; and the anion is 1,3-benzodioxol-5-oxide.

600. The composition of any one of claims 1-19, wherein the molecule is shogaol, which has CAS Registry Number 555-66-8; and the anion is 2-methoxy-4-(3-oxodec-4-enyl)phenolate.

601. The composition of any one of claims 1-19, wherein the molecule is silychristin, which has CAS Registry Number 33889-69-9; and the anion is selected from 3,7-dihydroxy-4-oxo-2-[7-hydroxy-3-(hydroxymethyl)-2-(4-hydroxy-3-methoxyphenyl)-2,3-dihydro-1-benzofuran-5-yl]-2,3-dihydro-4H-chromene-5-oxide; 3,5-dihydroxy-4-oxo-2-[7-hydroxy-3-(hydroxymethyl)-2-(4-hydroxy-3-methoxyphenyl)-2,3-dihydro-1-benzofuran-5-yl]-2,3-dihydro-4H-chromene-7-oxide; 3-(hydroxymethyl)-5-(3,5,7-trihydroxy-4-oxo-2,3-dihydro-4H-chromen-2-yl)-2-(4-hydroxy-3-methoxyphenyl)-2,3-dihydro-1-benzofuran-7-oxide; and 2-methoxy-4-[7-hydroxy-3-(hydroxymethyl)-5-(3,5,7-trihydroxy-4-oxo-2,3-dihydro-4H-chromen-2-yl)-2-(4-hydroxy-3-methoxyphenyl)-2,3-dihydro-1-benzofuran-2-yl]phenolate.

602. The composition of any one of claims 1-19, wherein the molecule is silydianin, which has CAS Registry Number 29782-68-1; and the anion is selected from 3,7-dihydroxy-4-oxo-2-{3-hydroxy-2-oxo-10-(4-hydroxy-3-methoxyphenyl)-4-oxatricyclo[4.3.1.0^{3,7}]dec-8-en-8-yl}-2,3-dihydro-4H-chromene-5-oxide; 3,5-dihydroxy-4-oxo-2-{3-hydroxy-2-oxo-10-(4-hydroxy-3-methoxyphenyl)-4-oxatricyclo[4.3.1.0³,7]dec-8-en-8-yl}-2,3-dihydro-4H-chromene-7-oxide; and 2-methoxy-4-{3-hydroxy-2-oxo-8-(3,5,7-trihydroxy-4-oxo-2,3-dihydro-4H-chromen-2-yl)-4-oxatricyclo[4.3.1.0^{3,7}]dec-8-en-10-yl}phenolate.

603. The composition of any one of claims 1-19, wherein the molecule is sinapyl alcohol, which has CAS Registry Number 537-33-7; and the anion is 2,6-dimethoxy-4-(3-hydroxyprop-1-enyl)phenolate.

604. The composition of any one of claims 1-19, wherein the molecule is sitostanyl ferulate, which has CAS Registry Number 117857-70-2; and the anion is 2-methoxy-4-(3-oxo-3-{[10,13-dimethyl-17-(5-isopropylhept-2-yl)-2,3,4,5,6,7,8,9,11,12,14,15,16,17-tetradecahydro-1H-cyclopenta[a]phenanthren-3-yl]oxy}prop-1-enyl)phenolate.

605. The composition of any one of claims 1-19, wherein the molecule is sitosteryl ferulate, which has CAS Registry Number 286011-30-1; and the anion is 2-methoxy-4-(3-oxo-3-{[10,13-dimethyl-17-(5-isopropylhept-2-yl)-2,3,4,7,8,9,11,12,14,15,16,17-dodecahydro-1H-cyclopenta[a]phenanthren-3-yl]oxy}prop-1-enyl)phenolate.

606. The composition of any one of claims 1-19, wherein the molecule is sophoradin, which has CAS Registry Number 23057-54-7; and the anion is selected from 3-hydroxy-2-isoprenyl-4-{1-oxo-3-[4-hydroxy-3,5-bis(isoprenyl)phenyl]prop-2-enyl}phenolate; 3-hydroxy-2-isoprenyl-6-{1-oxo-3-[4-hydroxy-3,5-bis(isoprenyl)phenyl]prop-2-enyl}phenolate; and 2,6-bis(isoprenyl)-4-[3-oxo-3-(2,4-dihydroxy-3-isoprenylphenyl)prop-1-enyl]phenolate.

607. The composition of any one of claims 1-19, wherein the molecule is sophoraflavanone G, which has CAS Registry Number 97938-30-2; and the anion is selected from 7-hydroxy-2-(2,4-dihydroxyphenyl)-4-oxo-8-(2-isopropenyl-5-methylhex-4-enyl)-2,3-dihydro-4H-chromene-5-oxide; 5-hydroxy-2-(2,4-dihydroxyphenyl)-4-oxo-8-(2-isopropenyl-5-methylhex-4-enyl)-2,3-dihydro-4H-chromene-7-oxide; 3-hydroxy-4-[5,7-dihydroxy-4-oxo-8-(2-isopropenyl-5-methylhex-4-enyl)-2,3-dihydro-4H-chromen-2-yl]phenolate; and 3-hydroxy-6-[5,7-dihydroxy-4-oxo-8-(2-isopropenyl-5-methylhex-4-enyl)-2,3-dihydro-4H-chromen-2-yl]phenolate.

608. The composition of any one of claims 1-19, wherein the molecule is sorbifolin, which has CAS Registry Number 23130-22-5; and the anion is selected from 6-hydroxy-7-methoxy-4-oxo-2-(4-hydroxyphenyl)-4H-chromene-5-oxide; 5-hydroxy-7-methoxy-4-oxo-2-(4-hydroxyphenyl)-4H-chromene-6-oxide; and 4-(5,6-dihydroxy-7-methoxy-4-oxo-4H-chromen-2-yl)phenolate.

609. The composition of any one of claims 1-19, wherein the molecule is spinacetin, which has CAS Registry Number 3153-83-1; and the anion is selected from 5,7-dihydroxy-6-methoxy-4-oxo-2-(4-hydroxy-3-methoxyphenyl)-4H-chromene-3-oxide; 3,7-dihydroxy-6-methoxy-4-oxo-2-(4-hydroxy-3-methoxyphenyl)-4H-chromene-5-oxide; 3,5-dihydroxy-6-methoxy-4-oxo-2-(4-hydroxy-3-methoxyphenyl)-4H-chromene-7-oxide; and 2-methoxy-4-(3,5,7-trihydroxy-6-methoxy-4-oxo-4H-chromen-2-yl)phenolate.

610. The composition of any one of claims 1-19, wherein the molecule is spinochrome B, which has CAS Registry Number 604-46-6; and the anion is selected from 3,6,7-trihydroxy-5,8-dioxonaphthalene-1-oxide; 4,6,7-trihydroxy-5,8-dioxonaphthalene-2-oxide; 3,5,7-trihydroxy-1,4-dioxonaphthalene-2-oxide; and 3,6,8-trihydroxy-1,4-dioxonaphthalene-2-oxide.

611. The composition of any one of claims 1-19, wherein the molecule is sterubin, which has CAS Registry Number 51857-11-5; and the anion is selected from 7-methoxy-4-oxo-2-(3,4-dihydroxyphenyl)-2,3-dihydro-4H-chromene-5-oxide; 2-hydroxy-4-(5-hydroxy-7-methoxy-4-oxo-2,3-dihydro-4H-chromen-2-yl)phenolate; and 2-hydroxy-5-(5-hydroxy-7-methoxy-4-oxo-2,3-dihydro-4H-chromen-2-yl)phenolate.

612. The composition of any one of claims 1-19, wherein the molecule is stigmasteryl ferulate, which has CAS Registry Number 20972-08-1; and the anion is 2-methoxy-4-(3-oxo-3-{[10,13-dimethyl-17-(5-isopropylhept-3-en-2-yl)-2,3,4,7,8,9,11,12,14,15,16,17-dodecahydro-1H-cyclopenta[a]phenanthren-3-yl]oxy}prop-1-enyl)phenolate.

613. The composition of any one of claims 1-19, wherein the molecule is stigmatellin, which has CAS Registry Number 91682-96-1; and the anion is 5,7-dimethoxy-3-methyl-4-oxo-2-(4,6-dimethoxy-3,5,11-trimethyltrideca-7,9,11-trienyl)-4H-chromene-8-oxide.

614. The composition of any one of claims 1-19, wherein the molecule is sudachitin, which has CAS Registry Number 4281-28-1; and the anion is selected from 7-hydroxy-6,8-dimethoxy-4-oxo-2-(4-hydroxy-3-methoxyphenyl)-4H-chromene-5-oxide; 5-hydroxy-6,8-dimethoxy-4-oxo-2-(4-hydroxy-3-methoxyphenyl)-4H-chromene-7-oxide; and 2-methoxy-4-(5,7-dihydroxy-6,8-dimethoxy-4-oxo-4H-chromen-2-yl)phenolate.

615. The composition of any one of claims 1-19, wherein the molecule is syringaresinol, which has CAS Registry Number 21453-69-0; and the anion is 2,6-dimethoxy-4-[(6-(4-hydroxy-3,5-dimethoxyphenyl)-1,3,3a,4,6,6a-hexahydrofuro[3,4-c]furan-3-yl]phenolate.

616. The composition of any one of claims 1-19, wherein the molecule is syringetin, which has CAS Registry Number 4423-37-4; and the anion is selected from 5,7-dihydroxy-4-oxo-2-(4-hydroxy-3,5-dimethoxyphenyl)-4H-chromene-3-oxide; 3,7-dihydroxy-4-oxo-2-(4-hydroxy-3,5-dimethoxyphenyl)-4H-chromene-5-oxide; 3,5-dihydroxy-4-oxo-2-(4-hydroxy-3,5-dimethoxyphenyl)-4H-chromene-7-oxide; and 2,6-dimethoxy-4-(3,5,7-trihydroxy-4-oxo-4H-chromen-2-yl)phenolate.

617. The composition of any one of claims 1-19, wherein the molecule is syringol, which has CAS Registry Number 91-10-1; and the anion is 2,6-dimethoxyphenolate.

618. The composition of any one of claims 1-19, wherein the molecule is taxifolin, which has CAS Registry Number 480-18-2; and the anion is selected from 3,7-dihydroxy-4-oxo-2-(3,4-dihydroxyphenyl)-2,3-dihydro-4H-chromene-5-oxide; 5,7-dihydroxy-4-oxo-2-(3,4-dihydroxyphenyl)-2,3-dihydro-4H-chromene-7-oxide; 2-hydroxy-4-(3,5,7-trihydroxy-4-oxo-2,3-dihydro-4H-chromen-2-yl)phenolate; and 2-hydroxy-5-(3,5,7-trihydroxy-4-oxo-2,3-dihydro-4H-chromen-2-yl)phenolate.

619. The composition of any one of claims 1-19, wherein the molecule is taxodone, which has CAS Registry Number 19039-02-2; and the anion is 9-hydroxy-2-isopropyl-4b,8,8-trimethyl-3-oxo-6,7,8a,9-tetrahydro-5H-phenanthrene-4-oxide.

620. The composition of any one of claims 1-19, wherein the molecule is techtochrysin, which has CAS Registry Number 520-28-5; and the anion is 7-methoxy-4-oxo-2-phenyl-4H-chromene-5-oxide.

621. The composition of any one of claims 1-19, wherein the molecule is thunberginol F, which has CAS Registry Number 147666-82-8; and the anion is selected from 1-oxo-3-[(3,4-dihydroxyphenyl)methylidene]-2-benzofuran-7-oxide; 2-hydroxy-4-[(7-hydroxy-1-oxo-2-benzofuran-3-ylidene)methyl]phenolate; and 2-hydroxy-5-[(7-hydroxy-1-oxo-2-benzofuran-3-ylidene)methyl]phenolate.

622. The composition of any one of claims 1-19, wherein the molecule is thunberginol G, which has CAS Registry Number 80394-88-3; and the anion is selected from 1-oxo-3-(3,4-dihydroxyphenyl)-3,4-dihydro-1H-isochromen-8-oxide; 2-hydroxy-4-(8-hydroxy-1-oxo-3,4-dihydro-1H-isochromen-3-yl)phenolate; and 2-hydroxy-5-(8-hydroxy-1-oxo-3,4-dihydro-1H-isochromen-3-yl)phenolate.

623. The composition of any one of claims 1-19, wherein the molecule is tinabinol, which has CAS Registry Number 50708-95-7; and the anion is 5,5-dimethyl-8-(3-methyloct-2-yl)-2,3-dihydro-1H-thiopyrano[2,3-c]chromene-10-oxide.

624. The composition of any one of claims 1-19, wherein the molecule is tinyatoxin, which has CAS Registry Number 58821-95-7; and the anion is 4-(2-oxo-2-{[(13-benzyl-6-hydroxy-4,17-dimethyl-5-oxo-15-isopropenyl-12,14,18-trioxapentacyclo[11.4.1.0^{1,10}.0^{2,6}.0^{11,15}]octadeca-3,8-dien-8-yl)methyl]oxy}ethyl)phenolate.

625. The composition of any one of claims 1-19, wherein the molecule is tithonine; and the anion is 2-methoxy-5-(7-methoxy-4-oxo-4H-chromen-2-yl)phenolate.

626. The composition of any one of claims 1-19, wherein the molecule is tricetin, which has CAS Registry Number 520-31-0; and the anion is selected from 7-hydroxy-4-oxo-2-(3,4,5-trihydroxyphenyl)-4H-chromene-5-oxide; 5-hydroxy-4-oxo-2-(3,4,5-trihydroxyphenyl)-4H-chromene-7-oxide; 2,3-dihydroxy-5-(5,7-dihydroxy-4-oxo-4H-chromen-2-yl)phenolate; and 2,6-dihydroxy-4-(5,7-dihydroxy-4-oxo-4H-chromen-2-yl)phenolate.

627. The composition of any one of claims 1-19, wherein the molecule is tricin, which has CAS Registry Number 520-32-1; and the anion is selected from 7-hydroxy-4-oxo-2-(4-hydroxy-3,5-dimethoxyphenyl)-4H-chromene-5-oxide; 5-hydroxy-4-oxo-2-(4-hydroxy-3,5-dimethoxyphenyl)-4H-chromene-7-oxide; and 2,6-dimethoxy-4-(5,7-dihydroxy-4-oxo-4H-chromen-2-yl)phenolate.

628. The composition of any one of claims 1-19, wherein the molecule is ubiquinol, which has CAS Registry Number 992-78-9; and the anion is selected from 4-hydroxy-2,3-dimethoxy-5-methyl-6-(3,7,11,15,19,23,27,31,35,39-decamethyltetraconta-2,6,10,14,18,22,26,30,34,38-decaenyl)phenolate and 4-hydroxy-2,3-dimethoxy-6-methyl-5-(3,7,11,15,19,23,27,31,35,39-decamethyltetraconta-2,6,10,14,18,22,26,30,34,38-decaenyl)phenolate.

629. The composition of any one of claims 1-19, wherein the molecule is urolithin A, which has CAS Registry Number 1143-70-0; and the anion is selected from 8-hydroxy-6-oxobenzo[c]chromene-3-oxide and 3-hydroxy-6-oxobenzo[c]chromene-8-oxide.

630. The composition of any one of claims 1-19, wherein the molecule is urolithin B, which has CAS Registry Number 1139-83-9; and the anion is 6-oxobenzo[c]chromene-3-oxide.

631. The composition of any one of claims 1-19, wherein the molecule is velutin, which has CAS Registry Number 25739-41-7; and the anion is selected from 7-methoxy-4-oxo-2-(4-hydroxy-3-methoxyphenyl)-4H-chromene-5-oxide and 2-methoxy-4-(5-hydroxy-7-methoxy-4-oxo-4H-chromen-2-yl)phenolate.

632. The composition of any one of claims 1-19, wherein the molecule is verrulactone A; and the anion is selected from 3,7-dihydroxy-9-methoxy-6-oxo-1-(2,3,7-trihydroxy-9-methoxy-6-oxobenzo[c]chromen-1-yl)benzo[c]chromene-2-oxide; 2,7-dihydroxy-9-methoxy-6-oxo-1-(2,3,7-trihydroxy-9-methoxy-6-oxobenzo[c]chromen-1-yl)benzo[c]chromene-3-oxide; and 2,3-dihydroxy-9-methoxy-6-oxo-1-(2,3,7-trihydroxy-9-methoxy-6-oxobenzo[c]chromen-1-yl)benzo[c]chromene-7-oxide.

633. The composition of any one of claims 1-19, wherein the molecule is verrulactone B; and the anion is selected from 3,7-dihydroxy-9-methoxy-6-oxo-1-(2,3,7-trihydroxy-9-methoxy-6-oxobenzo[c]chromen-4-yl)benzo[c]chromene-2-oxide; 2,7-dihydroxy-9-methoxy-6-oxo-1-(2,3,7-trihydroxy-9-methoxy-6-oxobenzo[c]chromen-4-yl)benzo[c]chromene-3-oxide; 2,3-dihydroxy-9-methoxy-6-oxo-1-(2,3,7-trihydroxy-9-methoxy-6-oxobenzo[c]chromen-4-yl)benzo[c]chromene-7-oxide; 3,7-dihydroxy-9-methoxy-6-oxo-4-(2,3,7-trihydroxy-9-methoxy-6-oxobenzo[c]chromen-1-yl)benzo[c]chromene-2-oxide; 2,7-dihydroxy-9-methoxy-6-oxo-4-(2,3,7-trihydroxy-9-methoxy-6-oxobenzo[c]chromen-1-yl)benzo[c]chromene-3-oxide; and 2,3-dihydroxy-9-methoxy-6-oxo-4-(2,3,7-trihydroxy-9-methoxy-6-oxobenzo[c]chromen-1-yl)benzo[c]chromene-7-oxide.

634. The composition of any one of claims 1-19, wherein the molecule is verrulactone E; and the anion is selected from 15,19,25,26-pentahydroxy-9,17-dimethoxy-3-methyl-5,14,21-trioxo-4,22-dioxaheptacyclo[14.10.2.0^{2,15}.0^{3,12}.0^{6,11}.0^{20,28}.0^{23,27}]octacosa-1(26),6(11),7,9,12,16,18,20(28),23(27),24-decaene-7-oxide; 7,15,25,26-pentahydroxy-9,17-dimethoxy-3-methyl-5,14,21-trioxo-4,22-dioxaheptacyclo[14.10.2.0^{2,15}.0^{3,12}.0^{6,11}.0^{20,28}.0^{23,27}]octacosa-1(26),6(11),7,9,12,16,18,20(28),23(27),24-decaene-19-oxide; 7,15,19,26-pentahydroxy-9,17-dimethoxy-3-methyl-5,14,21-trioxo-4,22-dioxaheptacyclo[14.10.2.0^{2,15}.0^{3,12}.0^{6,11}.0^{20,28}.0^{23,27}]octacosa-1(26),6(11),7,9,12,16,18,20(28),23(27),24-decaene-25-oxide; and 7,15,19,25-pentahydroxy-9,17-dimethoxy-3-methyl-5,14,21-trioxo-4,22-dioxaheptacyclo[14.10.2.0^{2,15}.0^{3,12}.0^{6,11}.0^{20,28}.0^{23,27}]octacosa-1(26),6(11),7,9,12,16,18,20(28),23(27),24-decaene-26-oxide.

635. The composition of any one of claims 1-19, wherein the molecule is wedelolactone, which has CAS Registry Number 524-12-9; and the anion is selected from 8,9-dihydroxy-3-methoxy-6-oxo-benzofurano[3,2-c]chromene-1-oxide; 1,9-dihydroxy-3-methoxy-6-oxo-benzofurano[3,2-c]chromene-8-oxide; and 1,8-dihydroxy-3-methoxy-6-oxo-benzofurano[3,2-c]chromene-9-oxide.

636. The composition of any one of claims 1-19, wherein the molecule is wighteone, which has CAS Registry Number 51225-30-0; and the anion is selected from 7-hydroxy-6-isoprenyl-4-oxo-3-(4-hydroxyphenyl)-4H-chromene-5-oxide; 5-hydroxy-6-isoprenyl-4-oxo-3-(4-hydroxyphenyl)-4H-chromene-7-oxide; and 4-(5,7-dihydroxy-6-isoprenyl-4-oxo-4H-chromen-3-yl)phenolate.

637. The composition of any one of claims 1-19, wherein the molecule is wightin; and the anion is selected from 7,8-dimethoxy-4-oxo-2-(3-hydroxy-2-methoxyphenyl)-4H-chromene-5-oxide and 2-methoxy-3-(5-hydroxy-7,8-dimethoxy-4-oxo-4H-chromen-2-yl)phenolate.

638. The composition of any one of claims 1-19, wherein the molecule is wogonin, which has CAS Registry Number 632-85-9; and the anion is selected from 7-hydroxy-8-methoxy-4-oxo-2-phenyl-4H-chromene-5-oxide and 5-hydroxy-8-methoxy-4-oxo-2-phenyl-4H-chromene-7-oxide.

639. The composition of any one of claims 1-19, wherein the molecule is xanthohumol, which has CAS Registry Number 6754-58-1; and the anion is selected from 5-hydroxy-6-isoprenyl-3-methoxy-2-[1-oxo-3-(4-hydroxyphenyl)prop-2-enyl]phenolate; 5-hydroxy-6-isoprenyl-3-methoxy-4-[1-oxo-3-(4-hydroxyphenyl)prop-2-enyl]phenolate; and 4-[3-oxo-3-(4,6-dihydroxy-5-isoprenyl-2-methoxyphenyl)prop-1-enyl]phenolate.

640. The composition of any one of claims 1-19, wherein the molecule is xanthomicrol, which has CAS Registry Number 16545-23-6; and the anion is selected from 6,7,8-trimethoxy-4-oxo-2-(4-hydroxyphenyl)-4H-chromene-5-oxide and 4-(5-hydroxy-6,7,8-trimethoxy-4-oxo-4H-chromen-2-yl)phenolate.

641. The composition of any one of claims 1-19, wherein the molecule is xylindein, which has CAS Registry Number 3779-11-1; and the anion is selected from 24-hydroxy-7,20-dipropyl-9,13,22,26-tetroxo-8,16,21,30-tetraoxaoctacyclo[15.11.1.14,28.02,15.03,12.05,10.018,23.025,29]triaconta-1,3,5(10),11,14,17(29),18(23),24,27-nonaene-11-oxide and 11-hydroxy-7,20-dipropyl-9,13,22,26-tetroxo-8,16,21,30-tetraoxaoctacyclo[15.11.1.14,28.02,15.03,12.05,10.018,23.025,29]triaconta-1,3,5(10),11,14,17(29),18(23),24,27-nonaene-24-oxide.

642. The composition of any one of claims 1-19, wherein the molecule is zapotinin, which has CAS Registry Number 14813-20-8; and the anion is 6-methoxy-4-oxo-2-(2,6-dimethoxyphenyl)-4H-chromene-5-oxide.

643. The composition of any one of claims 1-19, wherein the molecule is (C6)-CP 47,497, which has CAS Registry Number 132296-20-9; and the anion is 6-(3-hydroxycyclohexyl)-3-(2-methylhept-2-yl)phenolate.

644. The composition of any one of claims 1-19, wherein the molecule is (C7)-CP 47,497, which has CAS Registry Number 70434-82-1; and the anion is 6-(3-hydroxycyclohexyl)-3-(2-methyloct-2-yl)phenolate.

645. The composition of any one of claims 1-19, wherein the molecule is (C9)-CP 47,497, which has CAS Registry Number 134308-14-8; and the anion is 6-(3-hydroxycyclohexyl)-3-(2-methyldec-2-yl)phenolate.

646. The composition of any one of claims 1-19, wherein the molecule is [10]-dehydroshogaol; and the anion is 2-methoxy-4-(3-oxotetradec-1,4-dienyl)phenolate.

647. The composition of any one of claims 1-19, wherein the molecule is [10]-shogaol, which has CAS Registry Number 36752-54-2; and the anion is 2-methoxy-4-(3-oxotetradec-4-enyl)phenolate.

648. The composition of any one of claims 1-19, wherein the molecule is [12]-shogaol, which has CAS Registry Number 99742-10-6; and the anion is 2-methoxy-4-(3-oxohexadec-4-enyl)phenolate.

649. The composition of any one of claims 1-19, wherein the molecule is [4]-shogaol; and the anion is 2-methoxy-4-(3-oxooct-4-enyl)phenolate.

650. The composition of any one of claims 1-19, wherein the molecule is [8]-dehydroshogaol; and the anion is 2-methoxy-4-(3-oxododec-1,4-dienyl)phenolate.

651. The composition of any one of claims 1-19, wherein the molecule is [8]-shogaol, which has CAS Registry Number 36700-45-5; and the anion is 2-methoxy-4-(3-oxododec-4-enyl)phenolate.

652. The composition of any one of claims 1-19, wherein the molecule is 1,6-dihydroxyanthraquinone, which has CAS Registry Number 569-10-8; and the anion is selected from 6-hydroxy-9,10-dioxoanthracene-1-oxide and 5-hydroxy-9,10-dioxoanthracene-2-oxide.

653. The composition of any one of claims 1-19, wherein the molecule is 1,7-bis(4-hydroxyphenyl)-1,4,6-heptatrien-3-one, which has CAS Registry Number 149732-52-5; and the anion is selected from 4-[7-(4-hydroxyphenyl)-3-oxohepta-1,4,6-trieneyl]phenolate and 4-[7-(4-hydroxyphenyl)-5-oxohepta-1,3,6-trieneyl]phenolate.

654. The composition of any one of claims 1-19, wherein the molecule is 11-hydroxytetrahydrocannabinol, which has CAS Registry Number 36557-05-8; and the anion is 9-(hydroxymethyl)-6,6-dimethyl-3-pentyl-6a,7,8,10a-tetrahydro-6H-benzo[c]chromene-1-oxide.

655. The composition of any one of claims 1-19, wherein the molecule is 11-hydroxyyangonin, which has CAS Registry Number 77900-30-2; and the anion is 2-methoxy-5-[2-(4-methoxy-6-oxo-6H-pyran-2-yl)ethenyl]phenolate.

656. The composition of any one of claims 1-19, wherein the molecule is 11-methoxy-12-hydroxydehydrokavain; and the anion is 2-methoxy-4-[2-(4-methoxy-6-oxo-6H-pyran-2-yl)ethyl]phenolate.

657. The composition of any one of claims 1-19, wherein the molecule is 11-nor-9beta-hydroxyhexahydrocannabinol, which has CAS Registry Number 52171-85-4; and the anion is 9-hydroxy-6,6-dimethyl-3-pentyl-6a,7,8,9,10,10a-hexahydro-6H-benzo[c]chromene-1-oxide.

658. The composition of any one of claims 1-19, wherein the molecule is 3′,5′-di-tert-butyl-4′,7-dihydroxyflavone; and the anion is selected from 4-oxo-2-[3,5-bis(tert-butyl)-4-hydroxyphenyl]-4H-chromene-7-oxide and 2,6-bis(tert-butyl)-4-(7-hydroxy-4-oxo-4H-chromen-2-yl)phenolate.

659. The composition of any one of claims 1-19, wherein the molecule is 3,5,7-trihydroxy-8-isoprenyl-2-(4-methoxyphenyl)-4H-chromen-4-one, which is the aglycone of icariin, which has CAS Registry Number 489-32-7 and is described in The Merck Index monograph M4942; and the anion is selected from 5,7-dihydroxy-8-isoprenyl-4-oxo-2-(4-methoxyphenyl)-4H-chromene-3-oxide; and the anions of the plurality of anions consist of one, two, or each of 3,7-dihydroxy-8-isoprenyl-4-oxo-2-(4-methoxyphenyl)-4H-chromene-5-oxide; and 3,5-dihydroxy-8-isoprenyl-4-oxo-2-(4-methoxyphenyl)-4H-chromene-7-oxide.

660. The composition of any one of claims 1-19, wherein the molecule is 3-hydroxychrysazin, which has CAS Registry Number 52431-74-0; and the anion is selected from 4,5-dihydroxy-9,10-dioxoanthracene-2-oxide; 3,8-dihydroxy-9,10-dioxoanthracene-1-oxide; and 6,8-dihydroxy-9,10-dioxoanthracene-1-oxide.

661. The composition of any one of claims 1-19, wherein the molecule is 4′,7-dihydroxyflavone, which has CAS Registry Number 2196-14-7; and the anion is selected from 4-oxo-2-(4-hydroxyphenyl)-4H-chromene-7-oxide and 4-(7-hydroxy-4-oxo-4H-chromen-2-yl)phenolate.

662. The composition of any one of claims 1-19, wherein the molecule is 4-hydroxycoumarin, which has CAS Registry Number 1076-38-6; and the anion is 2-oxo-2H-chromene-4-oxide.

663. The composition of any one of claims 1-19, wherein the molecule is 4-hydroxyphenylacetaldehyde, which has CAS Registry Number 7339-87-9; and the anion is 4-(2-oxoethyl)phenolate.

664. The composition of any one of claims 1-19, wherein the molecule is 5-chloro-2-hydroxybenzophenone, which has CAS Registry Number 85-19-8; and the anion is 4-chloro-2-[(phenyl)carbonyl]phenolate.

665. The composition of any one of claims 1-19, wherein the molecule is 5-hydroxykavain; and the anion is 4-methoxy-6-oxo-2-(2-phenylethenyl)-6H-pyran-3-oxide.

666. The composition of any one of claims 1-19, wherein the molecule is 5-O-methylgenistein, which has CAS Registry Number 4569-98-6; and the anion is selected from 5-methoxy-4-oxo-3-(4-hydroxyphenyl)-4H-chromene-7-oxide and 4-(7-hydroxy-5-methoxy-4-oxo-4H-chromen-3-yl)phenolate.

667. The composition of any one of claims 1-19, wherein the molecule is 6-hydroxyflavone, which has CAS Registry Number 6665-83-4; and the anion is 2-phenyl-4-oxo-4H-chromene-6-oxide.

668. The composition of any one of claims 1-19, wherein the molecule is 6-hydroxyluteolin, which has CAS Registry Number 18003-33-3; and the anion is selected from 6,7-dihydroxy-4-oxo-2-(3,4-dihydroxyphenyl)-4H-chromene-5-oxide; 5,7-dihydroxy-4-oxo-2-(3,4-dihydroxyphenyl)-4H-chromene-6-oxide; 5,6-dihydroxy-4-oxo-2-(3,4-dihydroxyphenyl)-4H-chromene-7-oxide; 2-hydroxy-4-(5,6,7-trihydroxy-4-oxo-4H-chromen-2-yl)phenolate; and 2-hydroxy-5-(5,6,7-trihydroxy-4-oxo-4H-chromen-2-yl)phenolate.

669. The composition of any one of claims 1-19, wherein the molecule is 7,8-dihydroxyflavone, which has CAS Registry Number 38183-03-8; and the anion is selected from 8-hydroxy-4-oxo-2-phenyl-4H-chromene-7-oxide and 7-hydroxy-4-oxo-2-phenyl-4H-chromene-8-oxide.

670. The composition of any one of claims 1-19, wherein the molecule is 7-O-methylisoxanthohumol; and the anion is 4-(8-isoprenyl-5,7-dimethoxy-4-oxo-2,3-dihydro-4H-chromen-2-yl)phenolate.

671. The composition of any one of claims 1-19, wherein the molecule is 7-O-n-pentylisoxanthohumol; and the anion is 4-(8-isoprenyl-5-methoxy-4-oxo-7-pentyloxy-2,3-dihydro-4H-chromen-2-yl)phenolate.

672. The composition of any one of claims 1-19, wherein the molecule is 9-hydroxychrysarobin, which has CAS Registry Number 491-59-8; and the anion is selected from 8,9-dihydroxy-3-methylanthracene-1-oxide; 8,9-dihydroxy-6-methylanthracene-1-oxide; and 1,8-dihydroxy-3-methylanthracene-9-oxide.

673. The composition of any one of claims 1-19, wherein the molecule is sesaminol, which has CAS Registry Number 74061-79-3; and the anion is 6-[3-(1,3-benzodioxol-5-yl)-1,3,3a,4,6,6a-hexahydrofuro[3,4-c]furan-6-yl]-1,3-benzodioxol-5-oxide.

674. The composition of any one of claims 1-19, wherein the molecule is omeprazole, which has CAS Registry Number 73590-58-6 and is described in The Merck Index monograph M8209; and the anion is 6-methoxy-2-[(4-methoxy-3,5-dimethylpyridin-2-yl)methylsulfinyl]-1,3-benzimidazole.

675. The composition of any one of claims 1-19, wherein the molecule is psilocin, which has CAS Registry Number 520-53-6 and is described in The Merck Index monograph M9304; and the anion is 3-[2-(N,N-dimethylamino)ethyl]-1H-indol-4-oxide.

676. The composition of any one of claims 1-675, for use as a medicament.

677. The composition of any one of claims 1-676, wherein the composition is formulated for administration to a human; and the composition is formulated to convert the anion into the molecule ex vivo prior to the administration.

678. The composition of any one of claims 1-676, wherein the composition is formulated for administration to a human; and the composition is formulated to convert the anion into the molecule in situ subsequent to the administration.

679. A method to administer a bioactive agent to a subject, comprising providing a composition according to any one of claims 1-678, and administering the composition to the subject.

680. The method of claim 679, wherein the composition is formulated for oral administration, and the composition is formulated to allow the conversion of an anionic form of the bioactive agent into a molecular form of the bioactive agent before the bioactive agent reaches the stomach of the subject to allow absorption of the bioactive agent by the epithelial lining of the gastrointestinal tract between the lips and the stomach, excluding the stomach and the outer surfaces of the lips, and including the esophagus and the inner surfaces of the lips.
